(19) United States
(12) Patent Application Publication
(10) Pub. No.: US 2022/0387615 A1
(43) Pub. Date: Dec. 8, 2022

(54) COMPOSITIONS COMPRISING ENZYME CLEAVABLE LINKER PLATFORMS AND CONJUGATES THEREOF

(71) Applicant: Danmarks Tekniske Universitet, Kgs. Lyngby (DK)

(72) Inventors: Katrine Qvortrup, Kgs. Lyngby (DK); Anders Højgaard Hansen, Odense C (DK); Katja Egeskov Grier, København Ø (DK); Amalie Nørskov, Birkerød (DK)

(21) Appl. No.: 17/621,268

(22) PCT Filed: Jun. 26, 2020
(86) PCT No.: PCT/EP2020/068045

§ 371 Date: Dec. 21, 2021

(30) Foreign Application Priority Data
Jun. 26, 2019 (EP) 19182641.1

(51) Int. Cl. A61K 47/65; A61K 47/68; A61P 35/00

(52) U.S. Cl. CPC A61K 47/65; A61K 47/6809; A61P 35/00

(57) ABSTRACT

The present invention relates to a cleavable linker platform. In particular, the invention relates to construction of an enzyme cleavable linker platform conjugated to a drug or a diagnostically relevant compound, a biomolecule, and an enzyme cleavable group, for which cleavage of the enzyme cleavable group leads to release of the drug or diagnostically relevant compound.

### CROSS REFERENCE TO RELATED APPLICATIONS

This application is the U.S. National Stage of PCT/EP2020/068045 filed Jun. 26, 2020, which claims the priority of European Patent Application 19182641.1 filed Jun. 26, 2019, the entire contents of both are incorporated herein by reference in its entirety.

### TECHNICAL FIELD

The disclosure relates to a cleavable linker platform. In particular, the disclosure relates to an enzyme cleavable linker platform for which cleavage of an enzyme cleavable group leads to release of a drug or a diagnostically relevant compound.

### SUMMARY

The present disclosure presents a linker structure that attaches a drug in the form of a prodrug to both a targeting group and an enzyme cleavable group. We show, that the structure is stable under physiologically relevant conditions, but is cleaved upon exposure to various types of enzymes. We show, that upon cleavage, the prodrug is released as the active drug.

### DETAILED DESCRIPTION

Thus, an embodiment relates to a compound of formula (I),

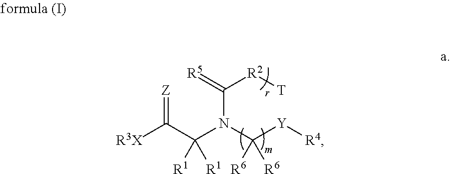

wherein:

each R¹ is independently selected from hydrogen, alkyl, substituted alkyl, aryl, substituted aryl, heteroaryl, or substituted heteroaryl or both R¹ together with the carbon to which they are attached form a cycloalkyl, substituted cycloalkyl, cycloheteroalkyl, or substituted cycloheteroalkyl;

R² is a bond or linking group;

R³ is a moiety comprising at least 19 atoms;

X is >N—, ═N—, —N(H)—, —O—, or —S—

Y is —O— or —N(R⁸)—; if Y is —O—, R³X is R³>N—, R³═N—, R³—N(H)—, R³—O—, or R³—S—; whereas if Y is —N(R⁸)—, R³X is R³—O— or R³—S—;

R⁴ is:

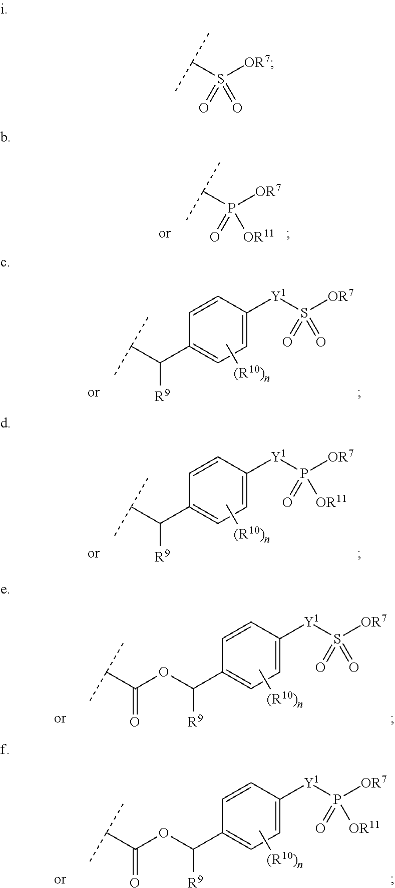

Y¹ is —O— or —N(R⁸)—;

R⁵ is ═O or ═NR¹²;

each R⁶ is independently selected from hydrogen, alkyl, substituted alkyl, aryl, substituted aryl, heteroaryl, or substituted heteroaryl, or any two R⁶ attached to the same carbon form a cycloalkyl, substituted cycloalkyl, cycloheteroalkyl, or substituted cycloheteroalkyl together with the carbon to which they are attached;

R⁷ is a negative charge or hydrogen;

each R⁸ is independently selected from hydrogen, alkyl, substituted alkyl, aryl, or substituted aryl;

R⁹ is selected from hydrogen, alkyl, substituted alkyl, aryl, or substituted aryl;

each R¹⁰ is independently selected from hydrogen, alkyl, substituted alkyl, aryl, or substituted aryl;

R¹¹ is a negative charge, hydrogen, alkyl, substituted alkyl, aryl, or substituted aryl;

R¹² is selected from hydrogen, alkyl, substituted alkyl, aryl, or substituted aryl;

Z is —O— or —S—;

T is a moiety containing at least 19 atoms;

m is 2 to 4;

n is 0 to 4;

r is 1 or more;

any R¹ is optionally together with any R⁶ a bond, an alkylene group, or a heteroalkylene group where such connection results in a cis-configuration of the substituent R³XC(═Z)— and the substituent —(C(R⁶)₂)ₖYR⁴ where k is 0 to m−1;

or a pharmaceutically acceptable salt thereof.

Another embodiment of the disclosure relates to a compound of formula (I),

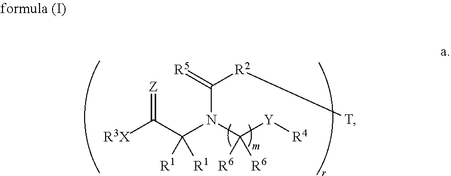

wherein:

each R¹ is independently selected from hydrogen, alkyl, substituted alkyl, aryl, substituted aryl, heteroaryl, or substituted heteroaryl or both R¹ together with the carbon to which they are attached form a cycloalkyl, substituted cycloalkyl, cycloheteroalkyl, or substituted cycloheteroalkyl;

R² is a bond or linking group;

R³ is a moiety comprising at least 19 atoms;

X is >N—, ═N—, —N(H)—, —O—, or —S—

Y is —O— or —N(R⁸)—; if Y is —O—, R³X is R³>N—, R³═N—, R³—N(H)—, R³—O—, or R³—S—; whereas if Y is —N(R⁸)—, R³X is R³—O— or R³—S—;

R⁴ is:

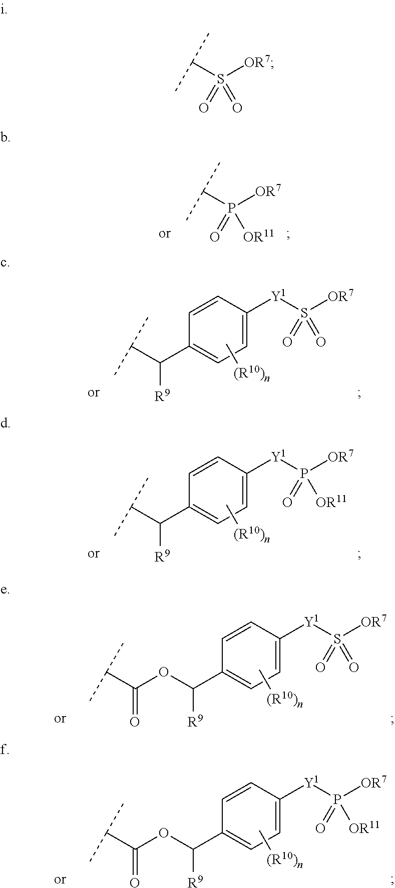

Y¹ is —O— or —N(R⁸)—;

R⁵ is ═O or ═NR¹²;

each R⁶ is independently selected from hydrogen, alkyl, substituted alkyl, aryl, substituted aryl, heteroaryl, or substituted heteroaryl, or any two R⁶ attached to the same carbon form a cycloalkyl, substituted cycloalkyl, cycloheteroalkyl, or substituted cycloheteroalkyl together with the carbon to which they are attached;

R⁷ is a negative charge or hydrogen;

each R⁸ is independently selected from hydrogen, alkyl, substituted alkyl, aryl, or substituted aryl;

R⁹ is selected from hydrogen, alkyl, substituted alkyl, aryl, or substituted aryl;

each R¹⁰ is independently selected from hydrogen, alkyl, substituted alkyl, aryl, or substituted aryl;

R¹¹ is a negative charge, hydrogen, alkyl, substituted alkyl, aryl, or substituted aryl;

R¹² is selected from hydrogen, alkyl, substituted alkyl, aryl, or substituted aryl;

Z is —O— or —S—;

T is a moiety containing at least 19 atoms;

m is 2 to 4;

n is 0 to 4;

r is 1 or more;

any R¹ is optionally together with any R⁶ a bond, an alkylene group, or a heteroalkylene group where such connection results in a cis-configuration of the substituent R³XC(═Z)— and the substituent —(C(R⁶)₂)ₖYR⁴ where k is 0 to m−1;

or a pharmaceutically acceptable salt thereof.

Another embodiment of the disclosure relates to a compound of formula (I),

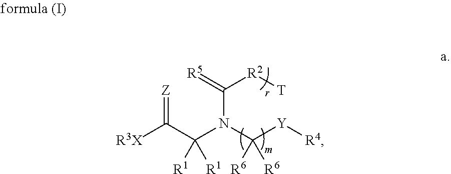

wherein:

each R¹ is independently selected from hydrogen, alkyl, substituted alkyl, aryl, substituted aryl, heteroaryl, or substituted heteroaryl or both R¹ together with the carbon to which they are attached form a cycloalkyl, substituted cycloalkyl, cycloheteroalkyl, or substituted cycloheteroalkyl;

R² is a bond or linking group;

R³ is a moiety comprising at least 19 atoms;

X is >N—, ═N—, —N(H)—, —O—, or —S—

Y is —O— or —N(R⁸)—; if Y is —O—, R³X is R³—O—, or R³—S—; whereas if Y is —N(R⁸)—, R³X is R³>N—, R³═N—, R³—N(H)—, R³—O— or R³—S—;

R⁴ is:

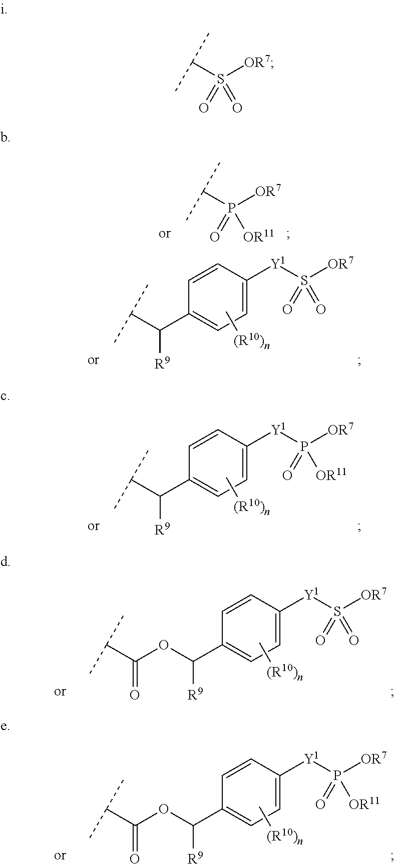

Y¹ is —O— or —N(R⁸)—;

R⁵ is ═O or ═NR¹²;

each R⁶ is independently selected from hydrogen, alkyl, substituted alkyl, aryl, substituted aryl, heteroaryl, or substituted heteroaryl, or any two R⁶ attached to the same carbon form a cycloalkyl, substituted cycloalkyl, cycloheteroalkyl, or substituted cycloheteroalkyl together with the carbon to which they are attached;

R⁷ is a negative charge or hydrogen;

each R⁸ is independently selected from hydrogen, alkyl, substituted alkyl, aryl, or substituted aryl;

R⁹ is selected from hydrogen, alkyl, substituted alkyl, aryl, or substituted aryl;

each R¹⁰ is independently selected from hydrogen, alkyl, substituted alkyl, aryl, or substituted aryl;

R¹¹ is a negative charge, hydrogen, alkyl, substituted alkyl, aryl, or substituted aryl;

R¹² is selected from hydrogen, alkyl, substituted alkyl, aryl, or substituted aryl;

Z is —O— or —S—;

T is a moiety containing at least 19 atoms;

m is 2 to 4;

n is 0 to 4;

r is 1 or more;

any R¹ is optionally together with any R⁶ a bond, an alkylene group, or a heteroalkylene group where such connection results in a cis-configuration of the substituent R³XC(═Z)— and the substituent —(C(R⁶)₂)ₖYR⁴ where k is 0 to m−1;

or a pharmaceutically acceptable salt thereof.

Yet another embodiment of the disclosure relates to a compound of formula (I),

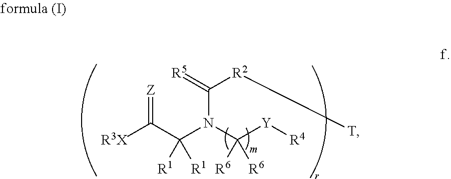

wherein:

each R¹ is independently selected from hydrogen, alkyl, substituted alkyl, aryl, substituted aryl, heteroaryl, or substituted heteroaryl or both R¹ together with the carbon to which they are attached form a cycloalkyl, substituted cycloalkyl, cycloheteroalkyl, or substituted cycloheteroalkyl;

R² is a bond or linking group;

R³ is a moiety comprising at least 19 atoms;

X is >N—, ═N—, —N(H)—, —O—, or —S—

Y is —O— or —N(R⁸)—; if Y is —O—, R³X is R³—O—, or R³—S—; whereas if Y is —N(R⁸)—, R³X is R³>N—, R³═N—, R³—N(H)—, R³—O— or R³—S—;

R⁴ is:

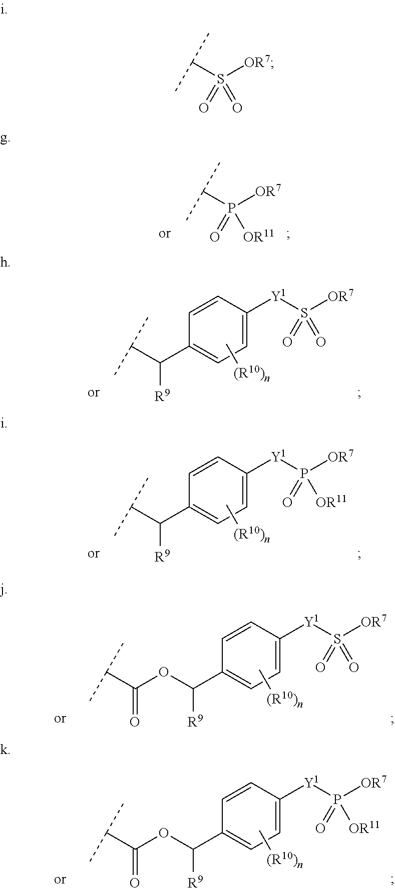

Y¹ is —O— or —N(R⁸)—;

R⁵ is ═O or ═NR¹²;

each R⁶ is independently selected from hydrogen, alkyl, substituted alkyl, aryl, substituted aryl, heteroaryl, or substituted heteroaryl, or any two R⁶ attached to the same carbon form a cycloalkyl, substituted cycloalkyl, cycloheteroalkyl, or substituted cycloheteroalkyl together with the carbon to which they are attached;

R⁷ is a negative charge or hydrogen;

each R⁸ is independently selected from hydrogen, alkyl, substituted alkyl, aryl, or substituted aryl;

R⁹ is selected from hydrogen, alkyl, substituted alkyl, aryl, or substituted aryl;

each R¹⁰ is independently selected from hydrogen, alkyl, substituted alkyl, aryl, or substituted aryl;

R¹¹ is a negative charge, hydrogen, alkyl, substituted alkyl, aryl, or substituted aryl;

R¹² is selected from hydrogen, alkyl, substituted alkyl, aryl, or substituted aryl;

Z is —O— or —S—;

T is a moiety containing at least 19 atoms;

m is 2 to 4;

n is 0 to 4;

r is 1 or more;

any R¹ is optionally together with any R⁶ a bond, an alkylene group, or a heteroalkylene group where such connection results in a cis-configuration of the substituent R³XC(═Z)— and the substituent —(C(R⁶)₂)ₖYR⁴ where k is 0 to m−1;

1. or a pharmaceutically acceptable salt thereof.

In a typical embodiment, the —Y—R⁴ group is an enzyme cleavable group and the R³X— group is a prodrug.

The following terms have the following meaning unless otherwise indicated. Any undefined terms have their art recognised meanings.

As used herein, the term “alkyl” by itself or as part of another substituent refers to a branched, or straight-chain monovalent hydrocarbon radical derived by the removal of one hydrogen atom from a single carbon atom of a parent alkane, alkene, alkyne, and the like. Where a specific degree of saturation is intended, the nomenclature “alkanyl”, “alkenyl”, and “alkynyl” is used. Typical alkyl groups include, but are not limited to, methyl, ethyl, propyls such as propan-1-yl or propan-2-yl; and butyls such as butan-1-yl, butan-2-yl, 2-methyl-propan-1-yl or 2-methyl-propan-2-yl, etc, and the like. In some embodiments, an alkyl group comprises from 1 to 20 carbon atoms. In other embodiments, an alkyl group comprises from 1 to 10 carbon atoms. In still other embodiments, an alkyl group comprises from 1 to 6 carbon atoms, such as from 1 to 4 carbon atoms.

“Alkanyl” by itself or as part of another substituent refers to a saturated branched, straight-chain or cyclic alkyl radical derived by the removal of one hydrogen atom from a single carbon atom of an alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methylpropan-1-yl (isobutyl), 2-methylpropan-2-yl (t-butyl), cyclobutan-1-yl, etc.; and the like.

“Alkylene” refers to a branched or unbranched saturated hydrocarbon chain, usually having from 1 to 40 carbon atoms, more usually 1 to 10 carbon atoms and even more usually 1 to 6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

“Alkenyl” by itself or as part of another substituent refers to an unsaturated branched, straight-chain or cyclic alkyl radical having at least one carbon-carbon double bond derived by the removal of one hydrogen atom from a single carbon atom of an alkene. The group may be in either the cis or trans conformation about the double bond(s). Typical alkenyl groups include, but are not limited to, ethenyl (vinyl); propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” by itself or as part of another substituent refers to an unsaturated branched, straight-chain or cyclic alkyl radical having at least one carbon-carbon triple bond derived by the removal of one hydrogen atom from a single carbon atom of an alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Acyl” by itself or as part of another substituent refers to a radical —C(O)R^{a}, where R^{a} is hydrogen, alkyl, cycloalkyl, cycloheteroalkyl, aryl, arylalkyl, heteroalkyl, heteroaryl, heteroarylalkyl as defined herein and substituted versions thereof. Representative examples include, but are not limited to formyl, acetyl, cyclohexylcarbonyl, cyclohexylmethylcarbonyl, benzoyl, benzylcarbonyl, piperonyl, succinyl, and malonyl, and the like.

“Alkoxy” by itself or as part of another substituent refers to a radical —OR^{b} where R^{b} represents an alkyl or cycloalkyl group as defined herein. Representative examples include, but are not limited to, methoxy, ethoxy, propoxy, butoxy, cyclohexyloxy and the like.

“Alkoxycarbonyl” by itself or as part of another substituent refers to a radical —C(O)OR^{c} where R^{c} represents an alkyl or cycloalkyl group as defined herein. Representative examples include, but are not limited to, methoxycarbonyl, ethoxycarbonyl, propoxycarbonyl, butoxycarbonyl, cyclohexyloxycarbonyl and the like.

“Aryl” by itself or as part of another substituent refers to a monovalent aromatic hydrocarbon radical derived by the removal of one hydrogen atom from a single carbon atom of an aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, pentacene, pentalene, pentaphene, perylene, phenalene, phenanthrene, picene, pleiadene, pyrene, pyranthrene, rubicene, triphenylene, trinaphthalene and the like. In certain embodiments, an aryl group comprises from 6 to 20 carbon atoms. In certain embodiments, an aryl group comprises from 6 to 12 carbon atoms. Examples of an aryl group are phenyl and naphthyl.

“Arylalkyl” by itself or as part of another substituent refers to an alkyl radical in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-Naphthylethan-1-yl, 2-Naphthylethen-1-yl, naphthobenzyl, 2-Naphthophenylethan-1-yl and the like. In certain embodiments, an arylalkyl group is C₇-C₃₀ arylalkyl, e.g., the alkyl, alkenyl or alkynyl moiety of the arylalkyl group is C₁-C₁₀ and the aryl moiety is (C₆-C₂₀). In certain embodiments, an arylalkyl group is C₇-C₂₀ arylalkyl, e.g., the alkyl, alkenyl or alkynyl moiety of the arylalkyl group is (C₁-C₈) and the aryl moiety is (C₆-C₁₂).

“Arylaryl” by itself or as part of another substituent, refers to a monovalent hydrocarbon group derived by the removal of one hydrogen atom from a single carbon atom of a ring system in which two or more identical or non-identical aromatic ring systems are joined directly together by a single bond, where the number of such direct ring junctions is one less than the number of aromatic ring systems involved. Typical arylaryl groups include, but are not limited to, biphenyl, triphenyl, phenylnaphthyl, binaphthyl, biphenylnapthyl, and the like. When the number of carbon atoms in an arylaryl group are specified, the numbers refer to the carbon atoms comprising each aromatic ring. For example, C₅-C₁₄ arylaryl is an arylaryl group in which each aromatic ring comprises from 5 to 14 carbons, e.g., biphenyl, triphenyl, binaphthyl, phenylnapthyl, etc. In certain embodiments, each aromatic ring system of an arylaryl group is independently a C₅-C₁₄ aromatic. In certain embodiments, each aromatic ring system of an arylaryl group is independently a C₅-C₁₀ aromatic ring. In certain embodiments, each aromatic ring system is identical, e.g., biphenyl, triphenyl, binaphthyl, trinaphthyl, etc.

“Cycloalkyl” by itself or as part of another substituent refers to a saturated or unsaturated cyclic alkyl radical. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane and the like. In certain embodiments, the cycloalkyl group is C₃-C₁₀ cycloalkyl. In certain embodiments, the cycloalkyl group is C₃-C₇ cycloalkyl.

“Cycloheteroalkyl” or “heterocyclyl” by itself or as part of another substituent, refers to a saturated or unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidine, quinuclidine and the like.

“Heteroalkyl, Heteroalkanyl, Heteroalkenyl and Heteroalkynyl” by themselves or as part of another substituent refer to alkyl, alkanyl, alkenyl and alkynyl groups, respectively, in which one or more of the carbon atoms (and any associated hydrogen atoms) are independently replaced with the same or different heteroatomic groups. Typical heteroatomic groups which can be included in these groups include, but are not limited to, —O—, —S—, —S—S—, —O—S—, —NR^{d}Rₑ—, ═N—N═, —N═N—, —N═N—NR^{f}R^{g} —PR^{h}—, —P(O)₂—, —P(O)R¹—, —OP(O)₂—, —S—O—, —S(O)—, —S(O)₂—, —SnR^{j}R^{k}— and the like, where R^{d}, R^{e}, R^{f}, R^{g}, R^{h}, R¹, R^{j} and R^{k} are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl” by itself or as part of another substituent, refers to a monovalent heteroaromatic radical derived by the removal of one hydrogen atom from a single atom of a heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, benzodioxole and the like. In certain embodiments, the heteroaryl group is from 5-20 membered heteroaryl. In certain embodiments, the heteroaryl group is from 5-10 membered heteroaryl. In certain embodiments, heteroaryl groups are those derived from thiophene, pyrrole, benzothiophene, benzofuran, indole, pyridine, quinoline, imidazole, oxazole and pyrazine.

“Heteroarylalkyl” by itself or as part of another substituent, refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heterorylalkynyl is used. In certain embodiments, the heteroarylalkyl group is a 6-30 membered heteroarylalkyl, e.g., the alkanyl, alkenyl or alkynyl moiety of the heteroarylalkyl is 1-10 membered and the heteroaryl moiety is a 5-20-membered heteroaryl. In certain embodiments, the heteroarylalkyl group is 6-20 membered heteroarylalkyl, e.g., the alkanyl, alkenyl or alkynyl moiety of the heteroarylalkyl is 1-8 membered and the heteroaryl moiety is a 5-12-membered heteroaryl.

“Aromatic Ring System” by itself or as part of another substituent, refers to an unsaturated cyclic or polycyclic ring system having a conjugated π electron system. Specifically included within the definition of “aromatic ring system” are fused ring systems in which one or more of the rings are aromatic and one or more of the rings are saturated or unsaturated, such as, for example, fluorene, indane, indene, phenalene, etc. Typical aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, pentacene, pentalene, pentaphene, perylene, phenalene, phenanthrene, picene, pleiadene, pyrene, pyranthrene, rubicene, triphenylene, trinaphthalene and the like.

“Heteroaromatic Ring System” by itself or as part of another substituent, refers to an aromatic ring system in which one or more carbon atoms (and any associated hydrogen atoms) are independently replaced with the same or different heteroatom.

Typical heteroatoms to replace the carbon atoms include, but are not limited to, N, P, O, S, Si, etc. Specifically included within the definition of “heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Typical heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Substituted” refers to a group in which one or more hydrogen atoms are independently replaced with the same or different substituent(s). Typical substituents include, but are not limited to, alkylenedioxy (such as methylenedioxy), -M, —R¹, ═O, —OR¹, —SR′, —S⁻, ═S, —CF₃, —CN, —OCN, —SCN, —NO, —NO₂, ═N₂, —N₃, —S(O)₂O⁻, —S(O)₂OH, —S(O)₂R¹, —OS(O)₂O⁻, —OS(O)₂R¹, —P(O)(O)₂, —P(O)(OR¹) (O), —OP(O)(OR¹)(OR^{m}), —C(O)R¹, —C(S)R¹, —C(O)OR¹, —C(O)NR¹R^{m}, —C(O)O⁻, —C(S)OR¹, —NRⁿC(O)NR¹R^{m}, —NRⁿC(S)NR¹R^{m}, (NR^{o})NR¹R^{m} and —C(NRⁿ)NR¹R^{m} where M is halogen; R¹, R^{m}, Rⁿ and R^{o} are independently hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl or substituted heteroaryl, or optionally R¹ and R^{m} together with the nitrogen atom to which they are bonded form a cycloheteroalkyl or substituted cycloheteroalkyl ring. In certain embodiments, substituents include -M, —R¹, ═O, —OR¹ —S⁻, ═S, —NR¹R^{m}, ═NR¹, —CF₃, —CN, —OCN, —SCN, —NO, —NO₂, ═N₂, —N₃, —S(O)₂R¹, —OS(O)₂O⁻, —OS(O)₂R¹, —P(O)(O⁻)₂, —P(O)(OR¹) (O), —OP(O)(OR¹)(OR^{m}), —C(O)R¹, —C(S)R¹, —C(O)OR¹, —C(O)NR¹R^{m}, —C(O)O⁻, —NRⁿC(O)NR¹R^{m}. In certain embodiments, substituents include -M, —R¹, ═O, —OR¹, SR¹, —NR¹R^{m}, —CF₃, —CN, —NO₂, —S(O)₂R¹, —P(O)(OR¹)(O⁻), —OP(O)(OR¹)(OR^{m}), —C(O)R¹, —C(O)OR¹, —C(O)NR¹R^{m}, —C(O)O⁻. In certain embodiments, substituents include -M, ═O, —OR¹, —SR¹, —NR¹R^{m}, —CF₃, —CN, —NO₂, —S(O)₂R¹, —OP(O)(OR¹)(OR^{m}), —C(O)R¹, —C(O)OR¹, —C(O)O⁻, where R¹, R^{m} and Rⁿ are as defined above. For example, a substituted group may bear a methylenedioxy substituent or one, two, or three substituents selected from a halogen atom, a C₁-C₄ alkyl group and a C₁-C₄ alkoxy group.

A hyphen (—) in the context of condensed molecular formulas indicates a single bond between the atom or group of atoms before the hyphen, and the atom or group of atoms after the hyphen. Examples include but are not limited to: N—H for a nitrogen/hydrogen single bond, C—O for at carbon/oxygen single bond, or C(═O)—O for a carbonyl carbon/oxygen single bond.

An equals sign (═) in the context of condensed molecular formulas indicates a double bond between the atom or group of atoms before the equals sign, and the atom or group of atoms after the equals sign. Examples include but are not limited to: C═O for a carbon/oxygen double bond, C(H)═C for at carbon/carbon double bond, or O═O for a oxygen/oxygen double bond.

A greater-than sign (>) in the context of condensed molecular formulas indicates two single bonds from two separate atoms of the group or groups of atoms before the greater-than sign to the atom after the greater-than sign. An example is R^{a}>NH, which indicates two single bonds from the group of atoms R^{a} to nitrogen. Accordingly, R^{a}>NH may be a cyclic compound such as piperidine, where R^{a} corresponds to the group of atoms —CH₂CH₂CH₂CH₂CH₂⁻. R^{a}>NH may alternatively be a non-cyclic compound such as ethylmethylamine, where R^{a} corresponds to the two groups of atoms —CH₂CH₃ and —CH₃.

such that R^{a}>N, indicates a cyclic compound, and in other cases indicates a straight-chain compound.

In the context of the present disclosure, an unmatched parenthesis in relation to chemical structures is taken to mean that the entire group inside the unmatched parenthesis is repeated the number of times specified by the index on the outside of the unmatched parenthesis.

The term “enzyme cleavable linker” and “enzyme cleavable linker platform” is used to refer to compounds of formula (I) and compounds of formula (II), and more specifically, to the molecular moiety linking R³/R¹⁰³, T/T¹⁰⁰, and R⁴/R¹⁰⁴.

After enzymatic cleavage between Y and R⁴, Y possesses a reactive lone-pair, and in certain cases a negative charge. Thus, Y is nucleophilic. The C═Z moiety is electrophilic and is thus subject to nucleophilic attack. Upon addition of Y to the C═Z double bond, a tetrahedral intermediate is formed (formula (III) below). Upon re-formation of the C═Z double bond, either Y or X can act as a leaving group to either re-form the linear linker motif (Y is leaving group) or form a ring-closed structure (X is leaving group) thus liberating R³X⁻. This mechanism is outlined in FIG. 2.

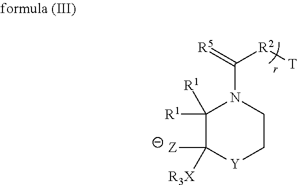

One embodiment of the invention relates to the compound of formula (III), wherein the meaning of the variables is the same as for formula I.

The nucleophilic Y⁻ is not necessarily obtained by direct enzymatic cleavage. In one embodiment, R⁴ is the moiety of formula IV or formula V. Upon hydrolysis by an aryl sulfatase, aryl sulfamidase, or aryl phosphatase, Y¹ is left as the negatively charged Y¹⁻ that self-immolates, leaving Y⁻ ready for ring closure as discussed above. The self-immolation mechanism is shown in FIG. 4.

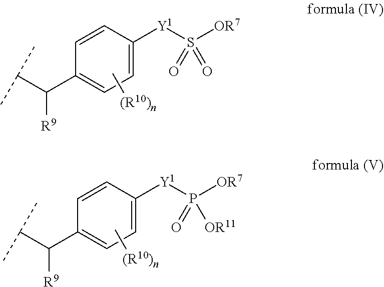

In one embodiment, R⁴ is the moiety of formula (VI) or formula (VII). Upon hydrolysis by an aryl sulfatase, aryl sulfamidase, or aryl phosphatase, Y¹ is left as the negatively charged Y¹⁻ that self-immolates leaving —YC(O)O⁻. Subsequently, the —YC(O)O⁻ moiety decarboxylates, leaving Y⁻ ready for ring closure as discussed above. The mechanism of this self-immolation is shown in FIG. 5.

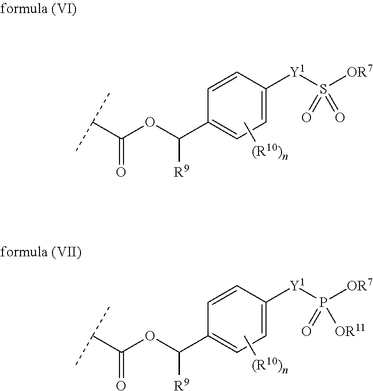

An advantage to the disclosed enzyme cleavable linker is that it distances R³ and the enzyme cleavable group YR⁴. This ensures that R³ will not sterically hinder the enzymatic activity at the enzyme cleavable site at YR⁴. This allows for conjugation of large and/or bulky R³ to the disclosed linkers to without affecting the rate of enzyme cleavage at the enzyme cleavable site at YR⁴.

One embodiment relates to the compound of formula (I) wherein the enzyme cleavable group —Y—R⁴ is cleaved by sulfatase or sulfamidase.

Examples of such structures are wherein Y is —O— or Y is —N(H)— and R⁴ is

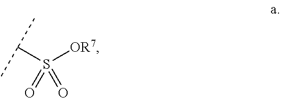

In a specific embodiment hereof, Y is O and the enzyme cleavable linker is cleaved by a sulfatase. Sulfatases are upregulated in afflicted cells during certain diseases such as cancer, and thus selective drug release is achieved. Sulfatase cleavable motifs have been shown to be stable in plasma for extended periods. It has also been shown that they are readily cleaved by appropriate sulfatases, for example Helix pomatia type H-1. Examples of sulfatases are sulfatase, cerebroside-sulfatase, steroid sulfatase, steryl-sulfatase, iduronate 2-sulfatase, N-acetylgalactosamine-6-sulfatase, N-sulfoglucosamine sulfohydrolase, glucosamine-6-sulfatase, N-sulfoglucosamine sulfohydrolase. A specific example is the sdsA1 sulfatase. Sdsa1 is a type III alkyl sulfatase expressed by Pseudomonas aeruginosa, a bacterium associated with multidrug resistance. The enzyme is responsible for sulfur scavenging from sodium dodecyl sulfate (SDS) as well as shorter alkyl sulfates.

In another specific embodiment hereof, Y is N and the bond between Y and R⁴ is cleaved by a sulfamidase. N-sulfamidase (E.C 3.10.1.1) is the only enzyme responsible for hydrolysing S—N bonds in humans and is a catalyst enabling heparin breakdown in lysosomes. (Sidhu et al., Acta Crystallogr D Biol Crystallogr, 2014, 70, 1321; Griffin et al., Protein Pept Lett., 2017, 24(8), 710). The lysosomal localisation and high specificity of this enzyme makes it an attractive candidate for ADC formats (Myette et al. J Biol Chem 2009, 284(5), 35189). Recent studies have reported higher activity levels of N-sulfamidase in patients suffering from chronic obstructive pulmonary disease, as well as mislocalisation of the enzyme from the lysosome (Weidner et al. Scientific Reports, 2009, 9).

One embodiment relates to the compound of formula (I) wherein the enzyme cleavable group —Y—R⁴ is cleaved following hydrolysis by aryl sulfatase or aryl sulfamidase.

Examples of such structures are wherein Y is —O— or Y is —N(H)— and R⁴ is

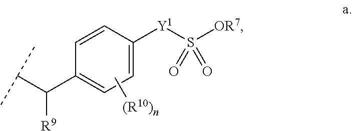

or wherein Y is —O— or Y is —N(H)— and R⁴ is

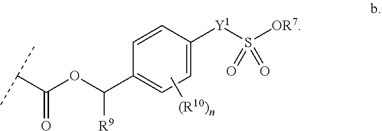

In both cases will the Y—R⁴ bond be cleaved after the self-immolation mechanism described previously.

In preferred embodiments, the substituents R^{m} are chosen such that the enzyme cleavable linker is stable in plasma for at least 4 days, but is cleaved by the corresponding aryl sulfatase or aryl sulfamidase when these enzymes are present. In an even more preferred embodiment, all R¹⁰ are hydrogen.

In a specific embodiment hereof, Y¹ is O, and the bond between Y¹ and S is cleaved by an aryl sulfatase. Such aryl sulfatase could be steroid sulfatase (STS). STS is responsible for hydrolysing steroid sulfates and is found mainly in steroidogenic and steroid-responsive tissue including prostate, ovaries and breast at low concentrations with higher levels in liver and adrenal gland tissues. Elevated expression levels of STS are found in oestrogen-dependent cancers including certain breast cancers, ovarian cancer and prostate cancer.

In another specific embodiment hereof, Y¹ is N, and the bond between Y¹ and S is cleaved by an aryl sulfamidase.

A specific embodiment of an aryl sulfatase (Y¹ is O) or aryl sulfamidase (Y¹ is N(H)) activated linker structure is formula (I) wherein R⁴ is

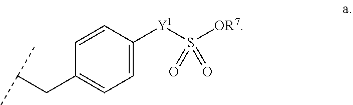

Another specific embodiment of a aryl sulfatase (Y¹ is O) or aryl sulfamidase (Y¹ is N(H)) activated linker structure is formula (I) wherein R⁴ is

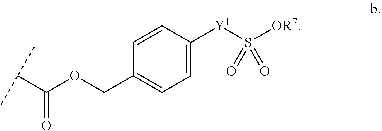

Yet another specific embodiment of the compound of formula (I) is wherein YR⁴ taken together is

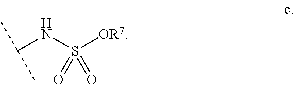

Preferably, R⁷ is H.

Yet another specific embodiment of the compound of formula (I) is wherein YR⁴ taken together is

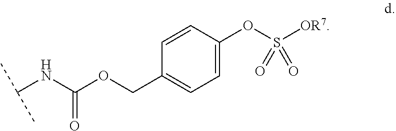

Preferably, R⁷ is H.

One embodiment relates to the compound of formula (I) wherein the enzyme cleavable group —Y—R⁴ is cleaved by a phosphatase, a phosphamidase, or an aryl phosphatase. Examples of such structures are formula (I) wherein Y is —O— or Y is —N(H)— and R⁴ is

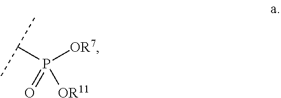

or wherein Y is —O— or Y —N(H)— and R⁴ is

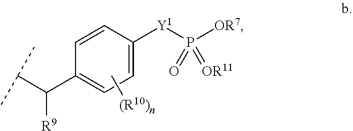

or wherein Y is —O— or Y is —N(H)— and R⁴ is

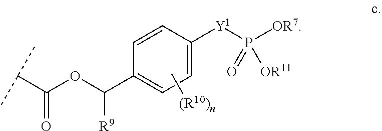

In preferred embodiments, the substituents R¹⁰ are chosen such that the enzyme cleavable linker is stable in plasma for at least 4 days, but is cleaved by an appropriate phosphamidase when these are present. In an even more preferred embodiment, all R¹⁰ are hydrogen.

In one embodiment of the present disclosure, Y is O and the enzyme cleavable linker is cleaved by a phosphatase. Examples of phosphatases are alkaline phosphatase, acidic phosphatase, phospholipase, phosphodiesterase, pyrophosphatase. A specific embodiment relates to R³X release in tissues with elevated human prostatic acid phosphatase (PAcP) activity. PAcP is an epithelium-specific antigen primarily present at high levels intracellularly in prostate cells (cPAcP, 0.5 mg/g wet prostate tissue) and in seminal fluids (sPAcP, 1 mg/mL). Elevated expression levels of sPAcP have been correlated with prostate cancer. Elevated serum PAcP levels are being used as a biomarker for prostate cancer diagnosis. Thus, R³X release as a diagnostic agent will indicate prostate cancer. R³X release as a drug, will selectively affect this tissue. Another specific example of an alkaline phosphatase is intestinal alkaline phosphatase (IAP). IAP is present throughout the gastrointestinal tract with the highest expression level in duodenum. Lower expression levels of IAP have been correlated with irritable bowel syndrome.

In another embodiment of the present disclosure, Y is N and the enzyme cleavable group is hydrolysed by a phosphamidase.

In an embodiment of the present disclosure, the bond between Y¹ and P is cleaved by an aryl phosphatase.

A specific embodiment of an aryl phosphatase activated linker structure is wherein Y is —O— or Y is —N(H)— and R⁴ is

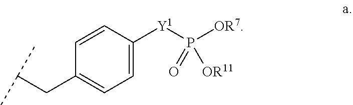

Another specific embodiment of an aryl phosphatase activated linker structure is formula (I) wherein Y is —O— or Y is —N(H)— and R⁴ is

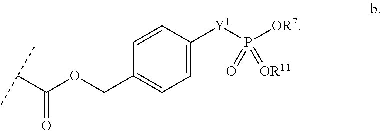

Yet another specific embodiment of the compound of formula (I) is wherein YR⁴ taken together is

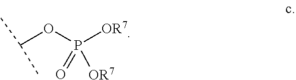

Preferably, R⁷ is H.

In one embodiment of the present disclosure, the enzyme cleavable group is conjugated to another enzyme cleavable group. An example of an enzyme cleavable group conjugated to another enzyme cleavable group is

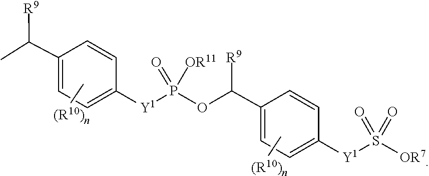

A linker conjugated to such an enzyme cleavable moiety would need cleavage by first an aryl sulfatase or aryl sulfamidase, and then an aryl phosphatase before R³XH is liberated from the enzyme cleavable linker.

In another embodiment of the present disclosure, Y—R⁴ is an enzyme cleavable group that is hydrolysed by a nuclease, and thus R⁴ comprises a nucleotide sequence. Examples of nucleases are DNase and RNase. In an embodiment, the property that nucleases can have a strong preference for either double-stranded or single-stranded nucleic acids is utilised. In a preferred embodiment of the present disclosure, R⁴ comprises a nucleotide sequence that is 15 to 100 nucleotides in length.

In an embodiment of the present disclosure, the bond between Y and R⁴ is cleaved by a glycosidase, and thus R⁴ comprises a mono-, oligo-, or polysaccharide. Glycosidases include, but are not limited to: glycosidase, β-galactosidase, α-galactosidase, α-(D-)-glucosidase, β-glucosidase, β-glucuronidase, α-mannosidase, N-acetyl-β-D-glucosaminidase, neuraminidase, cellulase, β-fucosidase, hyaluronidases.

In an embodiment of the present disclosure, the bond between Y and R⁴ is cleaved by a protease, and thus Y is N(R⁸) and R⁴ is a peptide sequence of 1 to 100 aminoacids. Proteases include, but are not limited to: dipeptidylpeptidases I, II and IV, plasminogen activator, calpain, elastase, trypsin, cathepsins B, C, L and O, urokinase, granzyme A, prostatin, thrombin, trypase, follipsin, kallikrein, plasmin, prohormone thiol protease, amyloid A4-generating enzymes, human adenovirus proteinase, kallikrein, HIV protease.

In an embodiment of the present disclosure, the bond between Y and R⁴ is cleaved by an esterase, and thus Y is S or O, and R⁴ is a substrate alkyl, aryl, substituted alkyl, or substituted aryl. Esterases include but are not limited to: cholinesterase, guanidinobenzoatase, thioesterases, lipases. Release of the R³X moiety in an way dependent on lipase endothelial lipase (LIPG) activity is advantageous as LIPG is biosynthesised in vascular endothelial cells with high metabolic rates. In addition, LIPG is found in liver, lung and kidney tissues where it is primarily situated near the cellular plasma membrane. Increased expression of LIPG levels correlates with pro-inflammatory markers, and high LIPG levels are found in type 2 diabetic patients. In patients with bronchial asthma, LIPG levels are elevated during eosinophilic inflammation in epithelial cells, alveolar type II cells, and lung endothelial cells. High LIPG have been reported in some testicular cancer, whilst down regulation of LIPG have been found in gastric cancer patient. Furthermore, LIPG has been shown to be upregulated in breast cancer and serve important roles in breast cancer proliferation.

One embodiment relates to the compound of formula (I) wherein both R¹ are the same and selected from hydrogen, methyl, ethyl, propyl, butyl, pentyl, hexyl, phenyl, or benzyl, or both R¹ taken together is —CH₂CH₂—.

In certain embodiments, R¹ are electron withdrawing groups which will accelerate the rate of ring closure because the electron density of the adjacent electrophilic centre (C═Z) is reduced. In other embodiments, R¹ are electron donating, which slows the rate of ring closure because the adjacent electrophilic centre is made more electron rich. In other embodiments, R¹ is large and will sterically hinder nucleophilic attack from Y on the electrophilic carbon of C═Z. In still other embodiments, le is small and will not hinder nucleophilic attack on C═Z, and the rate of ring closure is comparatively fast. In yet another embodiment of the present invention, the choice of R¹ will affect the rate of intramolecular ring closure due to the Thorpe-Ingold effect.

In an embodiment of the present disclosure, both R¹ are the same, thus avoiding achieving an achiral linker platform. An achiral linker platform provides a synthetic advantage in producing the linker platform, as resolution of enantiomers can be difficult, and because enantiomerically pure starting materials often are expensive. Employing an achiral linker may also be advantageous compared to a chiral linker, because regulatory affairs pertaining to chiral therapeutics can be challenging due to potertial adverse effects associated with enantiomers or diastereomers.

In a specific embodiment of the present disclosure both R¹ are hydrogen. Because hydrogen is a small substituent, intramolecular ring closure is comparatively fast.

In another specific embodiment of the present disclosure, both R¹ are methyl. Because methyl is a small substituent, intramolecular ring closure is comparatively fast.

In an embodiment of the present disclosure, both R¹ taken together is —CH₂CH₂—. Because —CH₂CH₂— is a substituent of intermediate size, and due to the unique molecular orbitals of the cyclopropane moiety, the rate of intramolecular ring closure is intermediate.

In another embodiment of the present disclosure, both R¹ are phenyl. Because phenyl is a large substituent, the rate of intramolecular ring closure is comparatively slow.

In an embodiment of the present disclosure, both R¹ are propyl. Because propyl is a large substituent, the rate of intramolecular ring closure is comparatively slow.

In an embodiment of the present disclosure, m is 2. When m is 2, the intramolecular ring closure reaction forms a six-membered ring. Six-membered rings generally form at a higher rate than seven- or eight-membered rings.

In an embodiment of the present disclosure, R⁶ is hydrogen, methyl, ethyl, propyl, butyl, pentyl, hexyl, phenyl, or benzyl, or, two R⁶ attached to the same carbon taken together is —CH₂CH₂—. The choice of R⁶ will affect the rate of intermolecular ring closure due to steric or electronic effects.

In a specific embodiment of the present disclosure, R⁶ is hydrogen. This allow the use of 2-aminoethanol as a synthetic starting material. The use of 2-aminoethanol is advantageous due to its inexpensiveness, which reduces overall costs of production.

In an embodiment of the present disclosure, two R⁶ group attached to the same carbon are identical, thus avoiding achieving an achiral linker platform.

An embodiment of the present disclosure relates to a compound of formula (I), wherein any one of R¹ is connected to any one of R⁶ with a bond, an alkylene group, or a heteroalkylene group, in such a way that it results in a cis-configuration of the substituent R³XC(═Z)— and the substituent —(C(R⁶)₂)ₖYR⁴ where k is 0 to m−1. Examples of such structures are given in FIG. 6.

The above embodiments details how the rate of ring closure and thereby the rate of release of drug can be adjusted by the choice of R¹, R⁶ and m to fit the particular needs. However, the rate of drug-release is also dependent on the election of X and Y. The first election criterion for X would be find an X on the drug, such that the attachment of X to the cleavable linker via X—C(═Z) inactivates the drug sufficiently. Likewise, the first election criterion for Y would be to match an enzyme that is active at the desired site of drug release. These first election criteria are then adjusted to achieve the desired speed of drug release, with X preferably being a similar or better leaving group than Y. For example, if Y is a nitrogen nucleophile, X is a sulfur, oxygen or nitrogen leaving group. If Y is an oxygen nucleophile, X is a sulfur or an oxygen leaving group. If fast drug release is desired, X is preferentially S or O, as S and O are better leaving groups than N.

One embodiment relates to the compound of formula (I) wherein R⁵ is O. In an embodiment the choice of R⁵ affects the hybridisation of the nitrogen atom bond to the R⁵C moiety. More specifically, the nature of the R⁵-carbon bond affects the hybridisation of said nitrogen atom. In some embodiments, the R⁵-carbon bond is a single bond, and the nitrogen atom is sp³ hybridised. I other embodiments, the R⁵-carbon bond is a double bond, and the nitrogen atom is sp³ hybridised. In a preferred embodiment, the R⁵-carbon bond is a double bond and nitrogen is sp² hybridised. The hybridisation of the nitrogen atom will in certain embodiment affect the rate of the ring-closure reaction.

In one embodiment of the present disclosure, R⁵ is oxygen, and R⁵═C—N taken together is a tertiary amide moiety. A tertiary amide moiety is especially advantageous for use in the present linker, because naturally occurring peptidases cannot cleave tertiary amides. Thus, the use of the tertiary amide moiety provides high in vivo stability of the enzyme cleavable linker. One embodiment relates to the compound of formula (I) wherein T is an antibody. By attaching an antibody to the disclosed enzyme cleavable linker, the compound will be concentrated at sites in the body with matching antigens. Thus, T provides a means of targeted delivery of the enzyme cleavable linker. Combined with the enzyme specificity elected for the enzyme cleavable group (—Y—R⁴), the drug (R³XH) will be released in sites with specific antigens and with activity of certain enzymes. In one embodiment of the present disclosure, T is an antibody, an antibody-derived antigen binding fragment (Fab), a single-chain variable fragment (scFv), a single-domain antibody (sdAb), a nanobody, a DARPin, a monobody, or an affibody, including the corresponding glycosylated variants and murine, humanized, and chimeric versions thereof. An antibody, also called an immunoglobulin (Ig), is large Y-shaped protein in the size range of roughly 150 kD with a well-defined quaternary structure. Antibodies are mainly produced by plasma B cells and function biologically to neutralize invading pathogens by binding to pathogen-specific antigens. T can be a bispecific antibody that may contain additional recombinantly or chemically inserted protein-based sequence motives to improve chemical or metabolic stability of said parent T, or to improve its targeting abilities and specificities e.g. by insertion of sortase A or transferrin receptor antibody.

T can be selected based on the ability to access and contact cellular targets. The biodistribution of antibodies and its derivables depends on the tissue type and can vary across the selected types. In general, the biodistribution has an indirect relationship between size and range of distribution. Given the large size of the antibody (10 nm), antibodies do not invade tissues as efficiently as smaller molecules, and its epitope must be situated towards the surface of a protein. When T is selected from the genetically engineered antibody derivables disclosed above, the compound of the disclosure can more easily penetrate tissues and access targets that are not necessarily located at the surface of a protein. Fab and scFv have molecular weights of about 55 and 28 kDa, respectively, and are in the size range of 7 and 3 nm. sdAb, nanobodies and DARPins are in the molecular weight range of 12-15 kDA and monobodies and affibodies have molecular weights of 10 and 6 kDa. These 5 molecules are all in the size range of about 3 nm. Being as small as 3 nm allows the molecules to target antigens located in crevices, such as near the active sites of many receptors.

In a preferred embodiment of the disclosure, T is an internalising antibody.

As outlined herein, targeted release of a conjugated cargo (R³X) is achieved via two mechanisms, namely: i) targeted delivery/uptake of the linker conjugate via the moiety T which ensures a higher concentration in the target cells compared to other cells of the subject and ii) release of cargo mediated by enzymes which may be up-regulated in the target cells compared to other cells of the subject. This mechanism of cargo delivery and release is especially advantageous if the cargo is a cytotoxic agent, because the conjugate carrying the cytotoxic agent is foremost delivered to the target cells and foremost cleaved within the target cells, releasing the cytotoxic agent. This is especially favourable in for instance chemotherapy, where adverse effects can be reduced significantly if only the afflicted cells in a subject are exposed to the cytotoxic agent. The mechanism is also advantageous if the cargo is a diagnostic agent, because the conjugate carrying the diagnostic agent is foremost delivered to the target cells and foremost cleaved within the target cells, releasing the diagnostic agent, thus providing for a higher concentration of the diagnostic agent within the targeted cells compared to the other cells of the subject.

In a further embodiment of the present disclosure, T is an antibody that is specific for disease relevant antigens including cancer specific antigens such as EGFR, HER2 GD2, PD1α, PDL1, RANKL, SLAMF7, antigens for autoimmune and inflammatory diseases (CD52, IL-1β, etc), cardiovascular diseases (PCSK9), infectious diseases (CD4) and osteoporosis (sclerostin). In an embodiment of the present disclosure, T is an antibody-derived antigen binding fragment (Fab) selected from the group consisting of: humanized kappa chain Fab that selectively binds to the anticoagulant dabigatran, chimeric IgG1 kappa-chain Fab acting as an glycoprotein IIb/IIIa receptor antagonist for use in individuals undergoing percutaneous coronary intervention, and humanized IgG1 kappa chain Fab acting as VEGFA inhibitor for treatment of neovasculation in patients suffering from macular oedema, diabetic retinopathy etc. In an embodiment of the present disclosure, T is a single-chain variable fragment (scFv) that is specific for antigens CD3 and CD19.

In a specific embodiment of the present disclosure, T is the antibody trastuzumab. This antibody binds to the HER2 receptor which is overexpressed in 20-30% of early-stage breast cancer patients. By binding to the receptor, internalization and downregulation is facilitated. Thus, the compound is internalized by the T moiety, whereby intracellular release of the drug is possible.

In a further embodiment, T is a nanobody such as 2Rs15d and structural analogues thereof that binds and activate the HER2 receptor to cause receptor-mediated endocytosis. In yet a further embodiment, T is an anti-human IgM Fab′2 able to induce B cell receptor clustering and internalization regardless of the internalization mechanism which depends on Fab concentration. In still another embodiment, T is a monobody that targets the Lck tyrosine kinase that in concert with Stx2B receptor Gb3 promote endocytosis of monobody.

In an embodiment of the present disclosure, T is a carbohydrate. The carbohydrate is a mono-, di-, oligo-, and poly-saccharides including the linear and branched versions thereof. The carbohydrates that contain more than one monosaccharide unit, which may be a hexose or a pentose, are chemically connected via S—, N—, C—, and O-glycosidic bonds of the type α-(1→2)—, α-(1→3)—, α-(1→4)—, α-(1→6)—, β-(1→2)—, β-(1→3)—, β-(1→4)—, and β-(1→6)—, or as epimeric mixtures. In one embodiment, the carbohydrate may be composed of monosaccharide components such as glucose, galactose, fructose and xylose, including in all cases the corresponding nitrogen, sulfur and phosphorous containing derivatives, and including in all cases deoxy-monosaccharide forms where tetrahydropyranyl defines the most deoxygenated monosaccharide. In the embodiment where T is composed of more than one monosaccharide, T may be composed of an all-carbohydrate structure or of multiple monosaccharides chemically connected via scaffolds of non-carbohydrate origin, such as peptidic.

In an embodiment of the present disclosure, T is D-mannose, which binds and recognizes the mannose receptor (CD206), highly expressed on specific cell types, including macrophages. Mannose based conjugates and other mannosylated drug carriers are advantageous for delivery of drugs to macrophages. In a preferred embodiment of the present disclosure, carbohydrate refers to N-Acetyl-D-Galactosylamine (GalNAc) based structures. GalNAc based structures binds and recognizes the high capacity receptor asialoglycoprotein receptor (ASGPR) abundantly expressed on hepatocytes. The ASGPR exhibits 50 times higher affinity for GalNAc than D-galactose. GalNAc multivalency is needed to reach nanomolar affinity on ASGPR.

In an embodiment of the present disclosure, T is a deoxyribonucleic acid (DNA) or a ribonucleic acid (RNA) based oligonucleotide, or a mixture thereof. The oligonucleotide is all-natural, and if not, contains either a phosphodiester backbone, a phosphorothioate backbone or a mixture thereof, and/or modifications on the 2′ position of the pentose sugar. In a further embodiment, T is an in vitro-selected single-stranded aptamer specifically recognizing disease or tissue specific antigens on the basis of its well-defined 3-dimensional structure. In a preferred embodiment, the aptamer specifically recognizes cell membrane receptor antigens to facilitate receptor mediated endocytosis. Further, the aptamer binds and recognizes tissue and/or disease specific antigens such as prostate-specific membrane antigen abundantly expressed in prostate cancer and in the vascular epithelium, CD4, HIV-1 envelope glycoprotein gp120, tenasin-C overexpressed in tumor stroma, EGFR, human protein tyrosine kinase 7, transferrin receptor, MUC1 receptor, and nucleolin.

In one embodiment of the present disclosure, T is a lipid. In some embodiments, the lipid is a natural lipid that is for example a fatty acyl, glycerolipid, glycerophospholipid, sphingolipid, saccharolipid, polyketide, sterol lipid or prenol lipids. In another embodiment, the lipid is a synthetic lipid accessible by chemical synthesis and chemically distinct from natural lipids.

In a further embodiment, T is a synthetic compound binding to and activating G protein-coupled receptors (GPCRs), such as agonists for peroxisome proliferator-activated receptors; PPARα, PPARγ, PPARδ, agoinsts for free fatty acid receptors (GPR); GPR40, GPR41, GPR43, and GPR120, and agonists for liver X receptos (LXR); LXRα and LXRβ. In a preferred embodiment, T can either facilitate internalisation once bound to the antigen presenting cell or can facilitate self-assembly. In some embodiments, T is cholesterol, a sterol lipid able to bind to the LDL receptor expressed in liver among other tissues to facilitate receptor mediated endocytosis. In some embodiments, T is the triterpene lipid squalene, a natural lipid used as excipient in pharmaceutical formulations and for delivery of oligonucleotides. In yet another preferred embodiment, T is agonistic in nature and/or can facilitate internalization once bound to the antigen presenting cell. In some embodiments, to achieve most efficient delivery of drug conjugates, T recognises receptor antigens that rapidly and continuously recycle back from the endosome back to the cellular membrane post cellular internalization, such as the LDL-receptor, asialoglycoprotein receptor (ASGPR), the transferrin receptor, the metabotropic glutamate receptor 5, the B cell receptor, the HER2 receptor and integrin receptors.

In one embodiment T is a cancer targeting group. Preferred cancer targeting groups are antibodies or fragments thereof that bind specifically to cancer cell markers. Cancer cell markers are biomolecules such as proteins, carbohydrates, glycoproteins, and the like that are exclusively or preferentially or differentially expressed on a cancer cell and thereby provide targets preferential or specific to the cancer. In various embodiments the preferential expression can be preferential expression as compared to any other cell in the organism, or preferential expression within a particular area of the organism (e.g within a particular organ or tissue). Examples of cancer cell markers are AFP, CA15-3 CA27-29, CA19-9, CA-125, calcitonin, calretinin, carcinoembryonic antigen, CD34, CD99MIC 2, CD117, chromagranin, chromosomes 3, 7, 17, and 9p21, cytokeratins, desmin, EMA, factor VIII, CD31 FL1, GFAP, GCDFP-15, HMB-45, hCG, inhibin, keratins, lymphocyte markers, MART-1, Myo D1, MSA, neurofilaments, NSE, FLAP, PSA, CD45, S100 protein, SMA, synaptophysin, thymidine kinase, Tumor M2-PK, and vimentin.

Above are illustrative examples of T as a biomolecule. In one embodiment R² is the chemical moiety that conjugates the enzyme cleavable linker to the biomolecule. In some embodiments a bioconjugation method is used for conjugating R² and T.

In an embodiment of the present disclosure, a non-selective conjugation method is used to conjugate the enzyme cleavable linker to T, such as a method that non-selectively conjugates on primary amines of T. In another embodiment, a selective conjugated method is used to conjugate the enzyme cleavable linker to T on specific functional groups on T. For example:
If the reactive functional group on T is a primary amine, reactive R² moieties can be chosen from N-hydroxysuccinimide esters, isothiocyanate or aldehyde;
If the reactive functional group on T is a carboxylate or a carboxylic acid, reactive R² moieties can be chosen from alcohol, hydrazine or amine;
If the reactive functional group on T is thiol, reactive R² moieties can be chosen from maleimide, α-halogenated carbonyl, or pyridyl disulfide;
If the reactive functional group on T is an alkyne, reactive R² moieties can be chosen from azide, and vice versa.

The reactive R² moieties such as N-hydroxysuccinimide esters, isothiocyanate, aldehyde, alcohol, hydrazine, amine, maleimide, α-halogenated carbonyl, pyridyl disulfide, alkyne, and azide may be especially useful because they are accessible synthetic intermediates. In a preferred embodiment, the reactive R² moiety is maleimide as shown in the examples herein. Maleimide is especially useful as a reactive group for conjugation.

In some embodiments of the present disclosure, r is 2 or more. I.e., more than one instance of the enzyme cleavable linker is conjugated to one molecule of T. In one embodiment, the multiple enzyme cleavable linkers conjugated to the same T are identical. In another embodiment, the enzyme cleavable linkers conjugated to the same T are different, for instance by having different R³ groups, releasing a plurality of drugs; having different R¹, such that release is at different rates; or even different YR⁴ moieties, such that drugs are released by several enzymes—or combinations thereof.

One embodiment relates to the compound of formula (I) wherein r is 1 to 12. It is preferred that r is selected from the group consisting of 1, 2, 3, 4, 5, 6, 7, 8, 9, 10, 11, and 12. Preferably, r is 3 or 4. In another embodiment, r is preferably 1 or 2.

In an embodiment of the present disclosure, R³X—H is a drug. The R³ moiety is conjugated to the enzyme cleavable linker via X, where X—H is a hydroxy group, a thiol group, a primary amine group, or a secondary amine group. The resulting moiety of the drug-linker-conjugate will in certain embodiments be an ester, a S-alkyl thioester, a secondary amide, or a tertiary amide.

For example, certain breast cancers are associated with upregulated phosphatase or sulfatase activity. To achieve linker activation, the enzyme cleavable group will be characterized in that Y is O. This leaves only S and O as X in the R³X leaving group.

In certain embodiments, the conjugated drug or have previously been shown useful in the treatment of a disease, disorder, or condition. In other embodiments, a person skilled in the art will have reason to believe that a certain compound will be useful in the treatment of a certain disease, disorder, or condition and will have a desire to utilise the disclosed enzyme cleavable linker platform for drug administration based on the properties on the disclosed enzyme cleavable linker platform. In yet another embodiment, a person skilled in the art will find it useful to employ the disclosed enzyme cleavable linker platform as a compound delivery system for compounds of clinical interest.

In an embodiment of the present disclosure, R³X—H is a diagnostically relevant compound. In one embodiment, the diagnostically relevant compound is a dye, for instance a fluorescent dye. In one embodiment, the fluorescent dye is resorufin. The fluorescence of resorufin is quenched upon conjugation to the enzyme cleavable linker. In this embodiment, the activity of a specific enzyme is monitored. Thus, the enzyme cleavable linker is conjugated to the dye resorufin and to an enzyme cleavable group specific to the enzyme under study. Upon cleavage of the enzyme cleavable group, the enzyme cleavable linker liberates the dye which, as the liberated dye, is fluorescent. This is a system for monitoring of the activity of the specific enzyme in vivo.

In an embodiment of the present invention, a fluorescent dye and a moiety T is conjugated to the enzyme cleavable group. In this embodiment, it is not known for which cells T facilitates internalisation. However, using the fluorescent properties of the dye, it is possible to assess for which cells T facilitates internalisation.

In preferred embodiments of the present disclosure, R³ has 19 or more atoms. That is, R³ has more than 20, such as 21, 22, 23, 24, 25 or even 26 atoms. In a preferred embodiment R³ is a small molecule, that is, R³ has a molecular weight below 2.000 daltons such as below 900 daltons.

In preferred embodiments of the present disclosure, the drug or diagnostically relevant compound possesses a lower pharmaceutical effect when conjugated to the enzyme cleavable linker compared to the drug or diagnostically relevant compound when not conjugated to the linker. That is, the compound of formula (I) is a prodrug. In a preferred embodiment, X is part of the pharmacophore of R³XH.

A preferred embodiment of the present disclosure provides for a compound of formula I wherein T is a cancer targeting group an R³X taken together with H as R³XH is a cytotoxic agent. In a further embodiment, the cytotoxic agent is querticin (CytI), PNU-159682 (CytII), camptothecin (CytIII), auristatin E (CytIV), or cryptothecin (CytV). In a preferred embodiment of the disclosure, T is an antibody that binds specifically to a cancer cell marker and R³X taken together with H as R³XH is querticin (CytI), PNU-159682 (CytII), camptothecin (CytIII), auristatin E (CytIV), or cryptothecin (CytV). In another preferred embodiment, T is a nanobody and R³XH is querticin (CytI), PNU-159682 (CytII), camptothecin (CytIII), auristatin E (CytIV), or cryptothecin (CytV). In another preferred embodiment of the disclosure, T is an antibody-derived antigen binding fragment, a single-chain variable fragment, a single-domain antibody, a DARPin, a monobody or an affibody, and R³X taken together with H as R³XH is querticin (CytI), PNU-159682 (CytII), camptothecin (CytIII), auristatin E (CytIV), or cryptothecin (CytV).

In an embodiment of the present disclosure, a compound disclosed herein is internalised into specific cells as dictated by the moiety T. Due to this selective internalisation, the compound disclosed herein appears in higher concentration within the cells where it is selectively internalised compared to cells where it is not selectively internalised.

In certain embodiments of the present disclosure, the enzyme cleavable linker is cleaved extracellularly. It follows that the conjugated drug or diagnostically relevant compound is released extracellularly.

In certain embodiments of the present disclosure, the enzyme cleavable linker is cleaved after internalisation into a cell. Following internalisation, the conjugated drug or diagnostically relevant compound is released intracellularly.

“Pharmaceutically acceptable salt” refers to a salt of a compound, which possesses the desired pharmacological activity of the compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methane sulfonic acid, ethanesulfonic acid, 1,2-ethane-disulfonic acid, 2-hydroxyethanesulfonic acid, benzenesulfonic acid, 4-chlorobenzenesulfonic acid, 2-Naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

In an embodiment of the present disclosure, the compound of formula (I) is administered to a patient in a therapeutically effective amount for treatment or prevention of a disease or a symptom. Another embodiment relates to the compound of formula (I) for use as a medicament.

As disclosed herein, the embodiments provide a composition, which comprises prodrugs based on enzyme cleavable linker platforms. The pharmaceutical composition according to the embodiments can further comprise a pharmaceutically acceptable carrier. The composition is conveniently formulated in a form suitable for oral (including buccal and sublingual) administration, for example as a tablet, capsule, thin film, powder, suspension, solution, syrup, dispersion, or emulsion. Alternatively, the composition is administered by intravenous, intramuscular, intrathecal, or subcutaneous injection, by rectal or vaginal insertion, by the ocular, otic, or nasal route, by inhalation or nebulisation, by the cutaneous route, or by transdermal delivery. The composition can contain components conventional in pharmaceutical preparations, e.g. one or more carriers, binders, lubricants, excipients (e.g., to impart controlled release characteristics), pH modifiers, sweeteners, bulking agents, colouring agents or further active agents.

In certain embodiments, the administration type is chosen as to achieve or assist in achieving a topical effect of treatment. In other certain embodiments, the administration type is chosen as to achieve or assist in achieving a systemic effect of treatment.

Compounds as described herein can be purified by any of the means known in the art, including recrystallisation, chromatographic means, such as high-performance liquid chromatography (HPLC), preparative thin layer chromatography, flash column chromatography, and ion exchange chromatography. For the chromatographic means, any suitable stationary phase can be used, including normal and reversed phases as well as ionic resins. See e.g. Introduction to Modern Liquid Chromatography, 2^{nd} Edition, ed. L. R. Snyder and J. J. Kirkland, John Wiley and Sons, 1979; and Thin Layer Chromatography, ed. E, Stahl, Springer-Verlag, New York, 1969.

Many general references providing commonly known chemical synthetic schemes and conditions useful for synthesizing the disclosed compounds are available (see e.g. Smith and March, March's Advanced Organic Chemistry: Reactions, Mechanisms, and Structure, 6^{th} edition, Wiley and Sons, Hoboken, N.J., 2007; or Vogel, A Textbook of Practical Organic Chemistry, Including Qualitative Organic Analysis, Fourth Edition, New York: Longman, 1978.

During any of the processes for preparation of the compounds of the present disclosure, it may be necessary and/or desirable to protect sensitive or reactive groups on any of the molecules concerned. This can be achieved by means of conventional protecting groups as described in standard works, such as T. W. Greene and P. G. M. Wuts, Protective Groups in Organic Synthesis, 4^{th} edition, Wiley, New York, 2006. The protecting groups can be removed at a convenient subsequent stage using methods known from the art.

The compounds described herein can contain one or more chiral centres and/or double bonds and therefore, can exist as stereoisomers, such as double-bond isomers (i.e. geometric isomers), enantiomers or diastereomers. Accordingly, all possible enantiomers and stereoisomers of the compounds including the stereoisomerically pure form (e.g. geometrically pure, enantiomerically pure, or diastereomerically pure) and enantiomeric and stereoisomeric mixtures are included in the description of the compounds herein. Enantiomeric and stereoisomeric mixtures can be resolved into their component enantiomers or stereoisomers using separation techniques or chiral synthesis techniques, or can be synthesised using enantiomerically pure or stereoisomerically pure precursors or reagents in stereoselective or stereospecific reactions well known to the skilled artisan.

The compounds described herein can also exist in several tautomeric forms, e.g. the enol form or the keto form, or mixtures thereof. Other molecular motifs capable of tautomerisation are well-known to persons skilled in the art. Accordingly, the chemical structures depicted herein encompass all possible tautomeric forms of the illustrated compounds.

The compounds described herein also include isotopically labelled compounds where one or more atoms have an atomic mass different from the atomic mass conventionally found in nature. Examples of isotopes that can be incorporated into the compounds disclosed herein include, but are not limited to, ²H, ³H, ¹¹C, ¹³C, ¹⁴C, ¹⁵N, ¹⁷O, ¹⁸O etc. Compounds can exist in unsolvated forms as well as solvated forms, including hydrated forms. In general, compounds can be hydrated or solvated. Certain compounds can exist in multiple crystalline or amorphous forms. In general, all physical forms are equivalent for the uses contemplated herein and are intended to be within the scope of the present disclosure.

An embodiment of the present disclosure is the compound of formula (II),

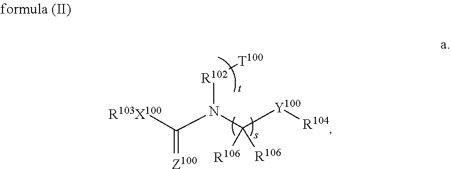

wherein:

R¹⁰² is a bond or linking group, analogous to R²;

R¹⁰³ is a moiety comprising at least 19 atoms, analogous to R³;

X¹⁰⁰ is —O— or —S—;

Y¹⁰⁰ is —O— or —N(R¹⁰⁸)—;

if Y¹⁰⁰ is —O—, R¹⁰³X¹⁰⁰ is R¹⁰³—O—, or R¹⁰³—S—, analogous to Y and X, whereas

if Y¹⁰⁰ is —N(R₁₀₈)—, R₁₀₃X₁₀₀ is R¹⁰³—O— or R¹⁰³—S—, analogous to Y and X; R¹⁰⁴ is:

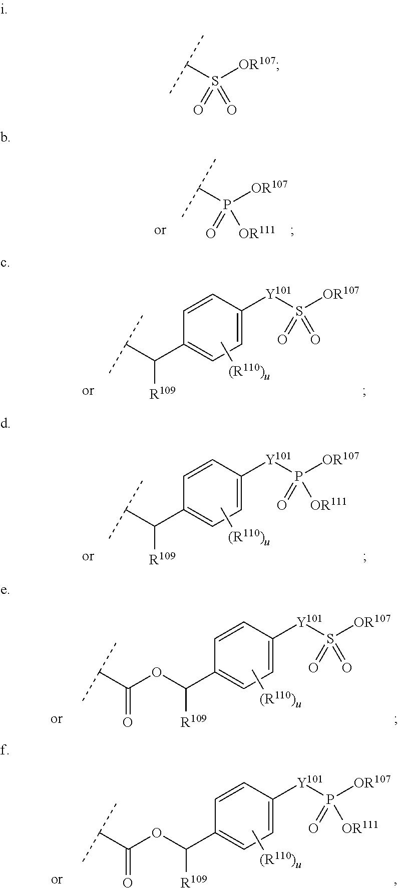

analogous to R⁴;

Y¹⁰¹ is —O— or —N(R¹⁰⁸)—, analogous to Y¹;

each R¹⁰⁶ is independently selected from hydrogen, alkyl, substituted alkyl, aryl, substituted aryl, heteroaryl, or substituted heteroaryl, or any two R⁶ attached to the same carbon form a cycloalkyl, substituted cycloalkyl, cycloheteroalkyl, or substituted cycloheteroalkyl together with the carbon to which they are attached, analogous to R⁶;

R¹⁰⁷ is a negative charge or hydrogen, analogous to R⁷;

each R¹⁰⁸ is independently selected from hydrogen, alkyl, substituted alkyl, aryl, or substituted aryl, analogous to R⁸;

R¹⁰⁹ is selected from hydrogen, alkyl, substituted alkyl, aryl, or substituted aryl;

each R¹¹⁰ is independently selected from hydrogen, alkyl, substituted alkyl, aryl, or substituted aryl, analogous to R¹⁰,

R¹¹¹ is a negative charge, hydrogen, alkyl, substituted alkyl, aryl, or substituted aryl, analogous to R¹¹;

Z¹⁰⁰ is —O— or —S—, analogous to Z;

T¹⁰⁰ is a moiety containing at least 19 atoms, analogous to T;

s is 2 or 3;

u is 0 to 4, analogous to n;

any R¹⁰⁶ is optionally taken together with another R¹⁰⁶ a bond, an alkylene group, or a heteroalkylene group;

or a pharmaceutically acceptable salt thereof.

After enzymatic cleavage between Y¹⁰⁰ and R¹⁰⁴, Y¹⁰⁰ possesses a reactive lone-pair, and in certain cases a negative charge. Thus, Y¹° ° is nucleophilic. The C═Z¹⁰⁰ moiety is electrophilic and is thus subject to nucleophilic attack. Upon addition of Y¹⁰⁰ to the C═Z¹⁰⁰ double bond, a tetrahedral intermediate is formed (formula (IV) or formula (V) below). Upon re-formation of the C═Z¹⁰⁰ double bond, either Y¹⁰⁰ or X¹⁰⁰ can act as a leaving group to either re-form the linear linker motif (Y¹⁰⁰ is leaving group) or form a ring-closed structure (X¹⁰⁰ is leaving group) thus liberating R¹⁰³(X¹⁰⁰)⁻. This mechanism is outlined in FIG. 3.

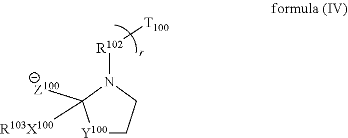

One embodiment of the invention relates to the compound of formula (IV) where the meaning of the variables is the same as for formula II.

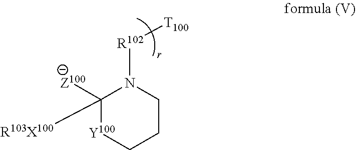

One embodiment of the invention relates to the compound of formula formula (V), where the meaning of the variables is the same as for formula II.

One preferred embodiment of the present disclosure relates to a compound of formula (II),

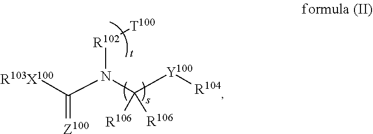

wherein R¹⁰³X¹⁰⁰ is R¹⁰³—O—, Z¹⁰⁰ is —O—, and Y₁₀₀ is —N(R¹⁰⁸)—. After the enzyme cleavable group Y¹⁰⁰—R¹⁰⁴ (corresponding to N(R¹⁰⁸)—R¹⁰⁴) is cleaved, Y¹⁰⁰ can perform a nucleophilic attack on the carbonyl group C═Z¹⁰⁰ (corresponding to C═O), which liberates R¹⁰³X¹⁰⁰ (corresponding to R¹⁰³O). This is a preferred embodiment because the nitrogen atom Y¹⁰° has sufficiently nucleophilicity to attack the carbonyl group C═Z¹⁰⁰, which is part of a carbamate. It is also a preferred embodiment of the disclosure because the oxygen of X¹⁰⁰, is a better leaving group than the nitrogen atom of Y¹⁰⁰, which favours liberation of the moiety R¹⁰³X¹⁰⁰ after Y¹⁰⁰ attacks the carbonyl group C═Z¹⁰⁰.

### DESCRIPTION OF FIGURES

FIG. 1: Overview of rates of intramolecular ring closure reaction for substrates 5A-E. The conditions are Amano lipase (ag/ml) in plasma at room temperature. The unit of the X axis of the graph is time. The unit of the y axis of the graph is % release.

FIG. 2: Mechanism of the intramolecular ring closure reaction for the compound of formula (I).

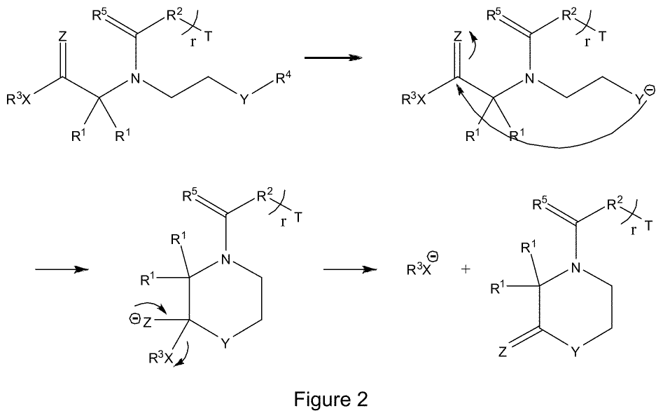

FIG. 3: Mechanism of the intramolecular ring closure reaction for the compound of formula (II).

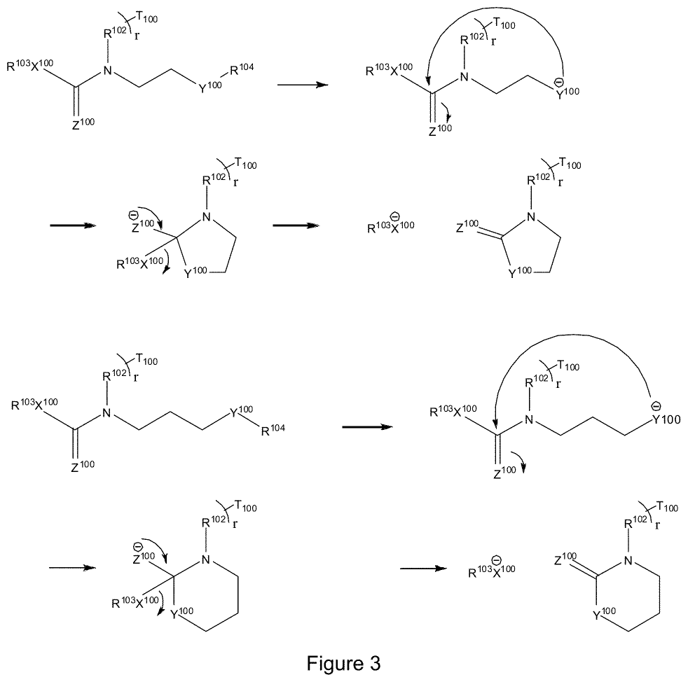

FIG. 4: Mechanism of the self-immolation of some embodiments of the compound of formula (I) and the compound of formula (II).

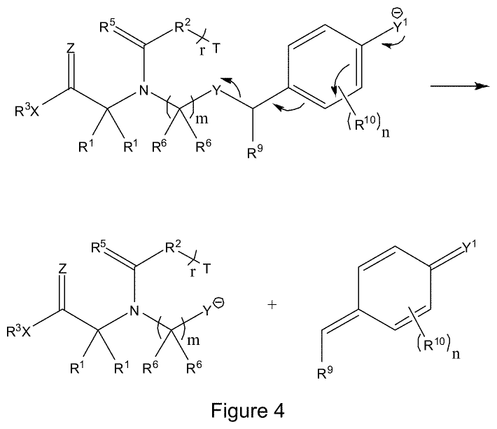

FIG. 5: Mechanism of the self-immolation of some embodiments of the compound of formula (I) and the compound of formula (II).

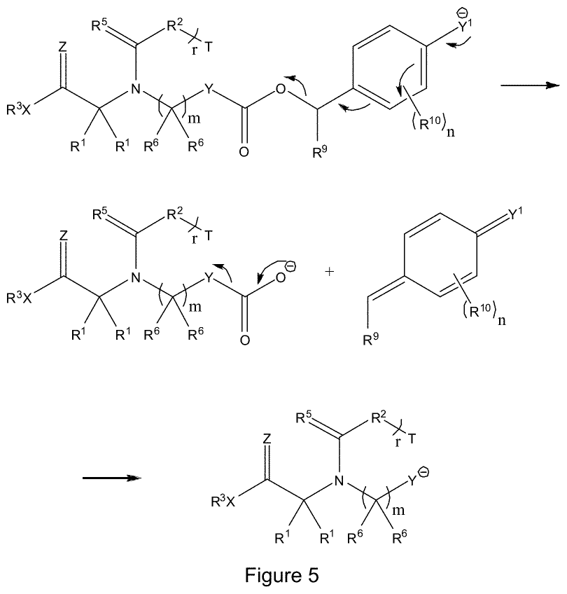

FIG. 6: Examples of the cis-criterion of the substituents after ring-closure. Substituents and the carbon atoms to which they are attached are shows in dashed boxes.

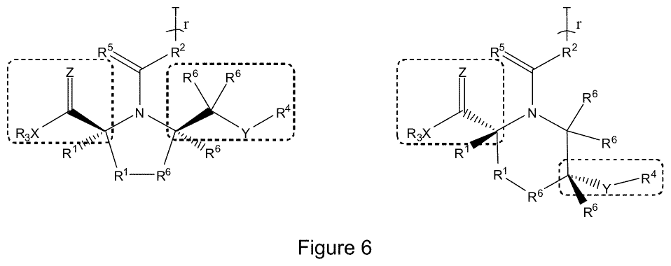

FIG. 7: An example of a synthetic pathway for producing a lipase cleavable linker relating to the compound of formula (I).

FIG. 8: An example of a synthetic pathway for producing a lipase cleavable linker relating to the compound of formula (I). 1-(4-bromophenyl)-2-hydroxyethan-1-one is used as a model drug.

FIG. 9: An example of a synthetic pathway for producing a lipase cleavable linker relating to the compound of formula (I) with a maleimide-based moiety for bioconjugation. The circled compound is a GLP-1 receptor allosteric modulator.

FIG. 10: An example of a synthetic pathway for producing a phosphatase cleavable linker relating to the compound of formula (I). The circled model drug mimics part of the structure of auristatin E, also shown in the figure.

FIG. 11: An example of a synthetic pathway for producing a phosphatase cleavable linker relating to the compound for formula (II). The circled moiety of the model drug mimics part of the structure of auristatin E, also shown in the figure.

FIG. 12: An example of a synthetic pathway for producing an aryl sulfatase cleavable linker relating to the compound of formula (II). The circled moiety of the model drug mimics part of the structure of auristatin E, also shown in the figure.

FIG. 13: An example of a synthetic pathway for producing a sulfamidase cleavable linker relating to the compound of formula (II). The circled moiety of the model drug mimics part of the structure of auristatin E, also shown in the figure.

FIG. 14: Structure of 2Rs15d-L-auristatin E drug conjugate and structure of Auristatin E drug conjugate.

FIG. 15: Synthetic pathway for producing a lipase cleavable linker relating to the compound of formula (I). 1-(4-bromophenyl)-2-hydroxyethan-1-one is used as a model drug.

FIG. 16: Overview of conditions for cleavage of compound X8 with a lipase with subsequent release of model drug X10 and formation of ring-closed structure X11.

FIG. 17: a) An example of LCMS UV trace of compound X8 in the presence of lipase at t=11 min. b) The results of the lipase-mediated release of X10 as observed by LCMS.

FIG. 18: Antibody drug conjugates with different enzyme cleavable linkers (phosphatase cleavable (PC), sulfatase cleavable (SC), sulfamidase cleavable (SAC), and lipase cleavable (LC) linkers) and cytotoxic agents (querticin (CytI), PNU-159682 (CytII), camptothecin (CytIII), auristatin E (CytIV), and cryptothecin (CytV)). The waved line indicates the site of conjugation between the enzyme cleavable linker and the cytotoxic agent.

FIG. 19: Sulfamidase cleavable camptothecin-based antibody-drug conjugates with different R-moieties that dictates the rate of release of camptothecin (a: R═H, b: R=Me, c: R=Et, d: R═Pr, e: R=Bn, f: R=Ph).

FIG. 20: Sulfamidase cleavable cryptothecin-based antibody-drug conjugates with different R-moieties that dictates the rate of release of cryptothecin (a: R═H, b: R=Me, c: R=Et, d: R═Pr, e: R=Bn, f: R=Ph).

FIG. 21: Sulfamidase cleavable antibody-drug conjugates comprising the model drug benzyl alcohol. Sulfamidase cleaves the sulfamide group, which forms a free amine. After cleavage of the sulfamide group, the free amine reacts on the carbamate carbonyl group, releasing the model drug.

### EXAMPLES

The following examples are put forth so as to provide those of ordinary skill in the art with a complete disclosure and description of how to make and use the embodiments.

### General Experimental Details

Reagents: All reagents were obtained from commercial sources and used without further purification.

Proton nuclear magnetic resonance (¹H-NMR) spectroscopy:

Proton nuclear magnetic resonance spectra were recorded using an internal deuterium lock (at 298 K unless stated otherwise) on Bruker DPX (400 MHz; 1H-13C DUL probe), Bruker Avance III HD (400 MHz; Smart probe), Bruker Avance III HD (500 MHz; Smart probe) and Bruker Avance III HD (500 MHz; DCH Cryoprobe) spectrometers. NMR spectra for compounds that appeared as mixtures of rotamers at 298 K are reported at a temperature that provided clear signals for unambiguous characterization. Chemical shifts (δH) are quoted in ppm to the nearest 0.01 ppm and are referenced to the residual non-deuterated solvent peak.

Carbon nuclear magnetic resonance (¹³C NMR) spectroscopy:

Carbon nuclear magnetic resonance spectra were recorded using an internal deuterium lock (at 298 K unless stated otherwise) on Bruker DPX (101 MHz), Bruker Avance III HD (101 MHz) and Bruker Avance III HD (126 MHz) spectrometers with broadband proton decoupling. Carbon spectra assignments are supported by DEPT editing, ¹H-¹³C HSQC or ¹H-¹³C HMBC spectra, or by analogy. Chemical shifts (SC) are quoted in ppm to the nearest 0.1 ppm and are referenced to the deuterated solvent peak. Data are reported as: chemical shift and assignment.

High resolution mass spectrometry (HMS):

Recorded on a Waters LCT Premier Time of Flight mass spectrometer. Reported mass values are within the error limits of ±5 ppm. ESI refers to the electrospray ionisation technique.

Liquid chromatography-mass spectrometry (LCMS):

LCMS analysis was performed on a Waters ACQUITY H-Class UPLC with an ESCi Multi-Mode Ionisation Waters SQ Detector 2 spectrometer using MassLynx 4.1 software; LC system: solvent A: 2 mMNH₄OAc in H₂O/MeCN (95:5); solvent B: MeCN; solvent C: 2% HCO₂H; gradient: A/B/C, 90:5:5-0:95:5 over 1 min at a flow rate of 0.6 mL min¹.

### Example 1: Synthesis of an Enzyme Cleavable Linker of Formula (I)

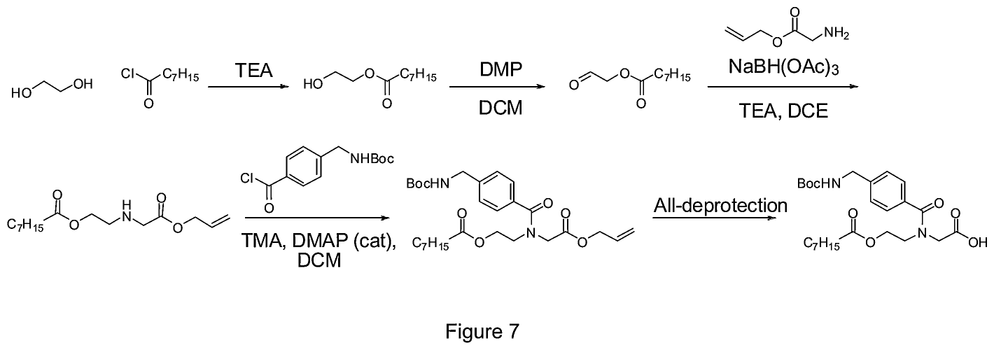

FIG. 7 shows a synthetic pathway for an enzyme cleavable linker of formula (I) comprising the synthetic procedures outlined below.

### 2-Hydroxyethyl octanoate

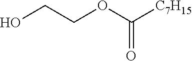

Octanoyl chloride (9.97 mL, 61.5 mmol) was added dropwise to a solution of ethylene glycol (34.25 mL, 615 mmol) and triethylamine (8.58 mL, 61.5 mmol) and stirred at rt overnight. The reaction mixture was quenched with water (150 mL) and extracted with CH₂Cl₂ (5×75 mL). The combined organic layers were washed with water (2×75 mL) and brine (75 mL), dried (MgSO₄), concentrated in vacuo and purified using a short plug of silica (0% to 5% MeOH in CH₂Cl₂) to yield the desired alcohol (5.7 g, 30.3 mmol, 49%) as a colourless oil.

R_{f}=0.24 (2.5% MeOH in CH₂Cl₂); vₘₐₓ (neat/cm⁻¹)=3453 (w, br, O—H), 2928 (m, C—H), 1736 (s, C═O), 1458, 1379; ¹H-NMR (400 MHz, DMSO-d₆) δ=4.77 (t, J=5.5 Hz, 1H), 4.01 (dd, J=5.8, 4.4 Hz, 2H), 3.55 (q, J=5.4 Hz, 2H), 2.28 (t, J=7.4 Hz, 2H), 1.52 (p, J=7.0 Hz, 2H), 1.26 (ddd, J=10.0, 5.2, 2.4 Hz, 8H), 0.91-0.79 (m, 3H); ¹³C NMR (101 MHz, DMSO-d₆) δ=172.98, 65.49, 58.97, 33.46, 31.12, 28.42, 28.36, 24.45, 22.04, 13.92; HRMS (ESI): m/z=189.15 [M+H]⁺, required m/z=189.1485 [C₁₀H₂₁O₃]⁺.

### 2-Oxoethyl octanoate

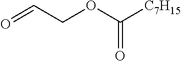

DMP (18.9 g, 44.5 mmol) was added to a solution of 2-Hydroxyethyl octanoate (5.59 g, 29.7 mmol) in CH₂Cl₂ (275 mL) at 0° C. The solution was stirred and allowed to slowly warm up to rt overnight. Reaction was diluted with 800 mL EtOAc and washed with H₂O (3×750 mL) followed by filtration. Organic phases washed with Na₂S₂O₃ (aq.) (3×500 mL), sat. NaHCO₃(aq.) (3×500 mL) and brine (2×500 mL). The combined organic layers were dried (MgSO₄), concentrated in vacuo and purified by flash column chromatography (3:7 EtOAc:heptane) to yield desired aldehyde (366 mg, 1.97 mmol, 74%) as a colourless oil.

R_{f}=0.30 (3:7 EtOAc:heptane); vₘₐₓ (neat/cm⁻¹)=2954 (m, C—H), 1737 (s, C═O), 1465, 1382; ¹⁻H NMR (400 MHz, Chloroform-d) δ=9.60 (s, 1H), 4.66 (s, 2H), 2.44 (t, J=7.5 Hz, 2H), 1.67 (p, J=7.5 Hz, 2H), 1.40-1.19 (m, 8H), 0.94-0.82 (m, 3H); ¹³C NMR (101 MHz, Chloroform-d) δ=195.97, 173.30, 68.62, 33.84, 31.76, 29.15, 29.02, 24.97, 22.72, 14.19; HRMS (ESI): m/z=187.13 [M+H]⁺, required 187.13 [C₁₀H₁₉O₃]⁺.

### 2-((2-(Allyloxy)-2-oxoethyl)amino)ethyl octanoate

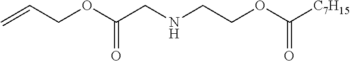

A suspension of triethylamine (1.35 mL, 9.66 mmol) and allyl glycinate (14.5 mmol) in DCE (5 mL) was stirred for 15 min before 2-oxoethyl octanoate (1.8 g, 9.66 mmol) and NaBH(OAc)₃ (4.10 g, 19.33 mmol) were added at rt. The stirred suspension was quenched after 18 h with sat. NaHCO₃(aq.) (150 mL) and extracted with CH₂Cl₂ (3×150 mL). The combined organic layers were dried (MgSO₄), concentrated in vacuo and purified by flash column chromatography (1:1 EtOAc:heptane) to yield desired amine (771 mg, 2.70 mmol, 28%) as a colourless oil.

R_{f}=0.17 (1:1 EtOAc:heptane); vₘₐₓ (neat/cm⁻¹)=3486 (w, br, N—H), 2925 (w, C—H), 1737 (s, C═O), 1645 (s, C═C), 1459; ¹H NMR (400 MHz, DMSO-d₆) δ=5.92 (ddt, J=17.3, 10.7, 5.4 Hz, 1H), 5.31 (dq, J=17.2, 1.7 Hz, 1H), 5.21 (dq, J=10.5, 1.4 Hz, 1H), 4.57 (dt, J=5.5, 1.5 Hz, 2H), 4.03 (t, J=5.7 Hz, 2H), 3.39 (s, 2H), 2.75 (t, J=5.7 Hz, 2H), 2.28 (t, J=7.4 Hz, 2H), 1.51 (p, J=7.2 Hz, 2H), 1.32-1.17 (m, 8H), 0.90-0.77 (m, 3H); ¹³C NMR (101 MHz, DMSO-d₆): δ=172.90, 171.84, 132.61, 117.77, 64.35, 63.60, 49.92, 46.94, 33.46, 31.10, 28.40, 28.34, 24.43, 22.02, 13.92; HRMS (ESI): m/z=286.2018 [M+H]⁺, required 286.2018 [C₁₅H₂₈NO₄]; UPLC-MS (ESI): m/z=286.6 [M+H]⁺, required 286.4 [C₁₅H₂₈NO₄]⁺.

### 2-(N-(2-(allyloxy)-2-oxoethyl)-4-(((tert-butoxycarbonyl)amino)methyl)benzamido)ethyl octanoate

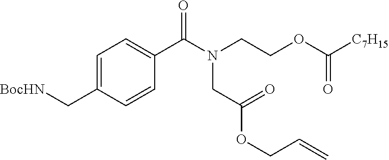

A suspension of 2-((2-(Allyloxy)-2-oxoethyl)amino)ethyl octanoate (468 mg, 1.64 mmol) and trimethylamine (298 μL, 2.13 mmol) in CH₂Cl₂ (6 mL) was cooled to 0° C. After 5 min tert-butyl (4-(chlorocarbonyl)benzyl)carbamate (575 mg, 2.13 mmol) and DMAP (cat. amount) were added and the mixture was allowed to reach rt. The stirred suspension was quenched after 18 h with H₂O (50 mL) and extracted with CH₂Cl₂ (2×50 mL). The combined organic layers were washed with 0.1M HCl (50 mL), sat. NaHCO₃(75 mL) and brin (75 mL), dried (MgSO₄), concentrated in vacuo and purified by flash column chromatography (1:1 EtOAc:heptane) to yield the desired product (351 mg, 0.68 mmol, 41%) as a colourless oil.

R_{f}=0.23 (1:1 EtOAc:heptane); ¹H NMR (400 MHz, DMSO-d₆): δ=7.88 (d, 2H, J=8.5 Hz), 7.75 (d, 2H, J=8.5 Hz), 7.46 (m, 3H), 7.38 (m, 2H), 5.46 (s, 2H), 4.34 (s, 2H), 4.21 (m, 2H), 3.67 (m, 2H), 2.28 (t, 2H, J=7.4 Hz), 1.55 (m, 2H), 1.28 (m, 8H), =0.87 (m, 3H); HRMS (ESI) m/z=546.1499 [M+H]⁺, required 546.1491 [C₂₇H₃₃NO₆Br]⁺.

### Example 2: Synthesis of an Enzyme Cleavable Linker of Formula (I)

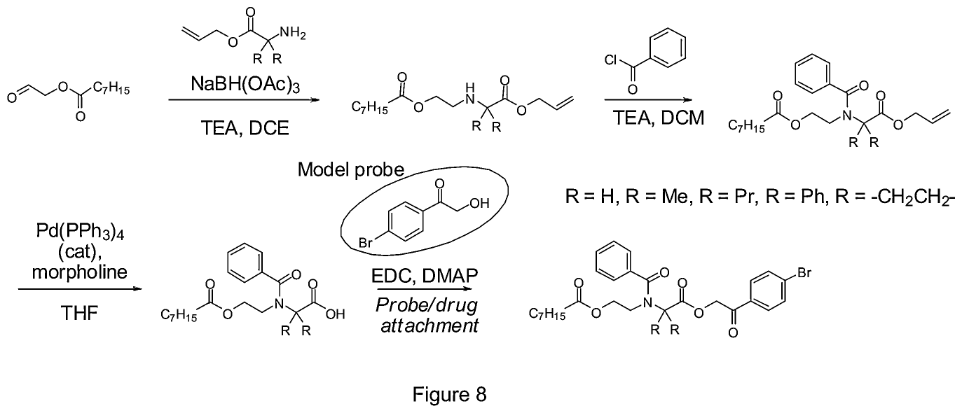

FIG. 8 shows a synthetic pathway for an enzyme cleavable linker of formula (I) comprising the synthetic procedures outlined below.

### 2-(N-(2-(allyloxy)-2-oxoethyl)benzamido)ethyl octanoate

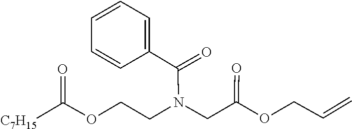

Benzoyl chloride (61.0 μL, 0.706 mmol) was added to a solution of triethylamine (98.4 μL, 0.706 mmol) and 2-((2-(Allyloxy)-2-oxoethyl)amino)ethyl octanoate (155 mg, 0.543 mmol) in CH₂Cl₂ (1 mL) at 0° C. The solution was warmed up to rt and stirred for 2 h before being quenched with water (10 mL) and extracted with CH₂Cl₂ (20 mL). The organic layer was washed with 1 M HCl (aq.) (10 mL), sat. NaHCO₃(aq.) (10 mL) and brine (10 mL). The organic layer was dried (MgSO₄), concentrated in vacuo and purified by flash column chromatography (25% EtOAc in PE) to yield the desired amide (125 mg, 0.321 mmol, 69%) as a colourless oil.

R_{f} 0.21 (25% EtOAc in PE); ¹H NMR (400 MHz, DMSO-d₆) δ=7.45 (m, 3H), 7.35 (m, 2H), 5.92 (m, 1H), 5.31 (d, 1H, J=17.6 Hz), 5.23 (d, 1H, J=10.6 Hz), 4.62 (d, 2H, J=5.0 Hz), 4.20 (m, 4H), 3.64 (m, 2H), 2.27 (t, 2H, J=7.3 Hz), 1.54 (m, 2H), 1.28 (m, 8H), 0.88 (m, 3H); 6C (126 MHz, DMSO-d₆, 373K) 172.0, 170.8, 168.6, 135.4, 131.7, 128.9, 127.8, 125.6, 117.4, 64.4, 61.8, 60.8, 44.8, 33.0, 30.4, 27.8, 27.6, 23.8, 21.3, 13.0; HRMS (ESI) m/z found 412.2105 [M+Na]⁺, required 412.2100 [C₂₂H₃₁NO₅Na]⁺.

### N-benzoyl-N-(2-(octanoyloxy)ethyl)glycine

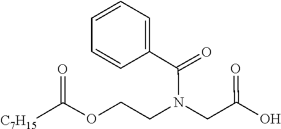

Tetrakis(triphenylphosphine)palladium(O) (20.6 mg, 17.8 μmol) and allyl ester 2-(N-(2-(allyloxy)-2-oxoethyl)benzamido)ethyl octanoate (139 mg, 0.357 mmol) were dissolved in THF (20 mL) and stirred at rt for 5 min. Morpholine (308 μL, 3.57 mmol) was added dropwise to the yellow solution and stirred for 4 h at rt. The resulting green solution was concentrated in vacuo and diluted with ether (10 mL) before being washed with 1 M HCl (aq.) (3×10 mL). The organic layer was filtered through a bed of celite washing thoroughly with ether, dried (MgSO₄), concentrated in vacuo and purified by flash column chromatography (0.5% acetic acid in 2:1 EtOAc/PE) to yield the desired acid (114 mg, 0.327 mmol, 91%) as a pale yellow oil.

Rf 0.39 (0.1% acetic acid in EtOAc); vmax (neat/cm-1) 3000 (w, br, O—H), 2928 (w, C—H), 1733 (s, C═O), 1643 (s, C═O), 1598 (m, C═C), 1575 (m, C═C), 1498, 1460; 6H (500 MHz, DMSO-d6, 373K) 7.43 (m, 3H), 7.35 (m, 2H), 4.19 (m, 2H), 4.07 (s, 2H), 3.63 (m, 2H), 2.28 (t, 2H, J=7.4 Hz), 1.55 (m, 2H), 1.29 (m, 8H), 0.88 (m, 3H); 6C (126 MHz, DMSO-d₆, 373K) 172.0, 170.7, 169.6, 135.7, 128.8, 127.8, 125.9, 63.8, 60.8, 47.6, 33.0, 30.4, 27.8, 27.6, 23.8, 21.2, 13.0; HRMS (ESI) m/z found 372.1773 [M+Na]⁺, required 372.1781 [C19H27NO5Na]⁺.

### 2-(N-(2-(2-(4-bromophenyl)-2-oxoethoxy)-2-oxoethyl)benzamido)ethyl octanoate

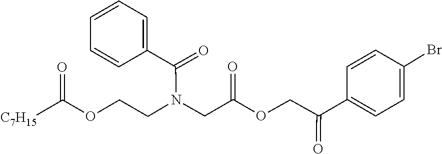

EDC (89.7 mg, 0.468 mmol) was added to a solution of acid N-benzoyl-N-(2-(octanoyloxy)ethyl)glycine (109 mg, 0.312 mmol), DMAP (57.2 mg, 0.468 mmol) and 1-(4-bromophenyl)-2-hydroxyethan-1-one (101 mg, 0.468 mmol) at 0° C. The suspension was warmed up to rt and stirred for 3 h before being diluted with CH2Cl2 (10 mL) and washed with sat. NaHCO₃(aq.) (10 mL) and 1 M HCl (aq.) (10 mL). The organic layer was dried (MgSO₄), concentrated in vacuo and purified by flash column chromatography (25% EtOAc in PE) to yield 2-(N-(2-(2-(4-bromophenyl)-2-oxoethoxy)-2-oxoethyl)benzamido)ethyl octanoate (97.0 mg, 0.177 mmol, 57%) as a colourless oil.

Rf 0.19 (25% EtOAc in PE); vmax (neat/cm-1) 2929 (w, C—H), 1735 (s, C═O), 1705 (s, C═O), 1640 (s, C═C), 1586 (s, C═C), 1455; 6H (500 MHz, DMSO-d₆): 7.88 (d, 2H, J=8.5 Hz, H3), 7.75 (d, 2H, J=8.5 Hz, H2), 7.46 (m, 3H, H21 and H23), 7.38 (m, 2H, H22), 5.46 (s, 2H, H6), 4.34 (s, 2H, H8), 4.21 (m, 2H, H10), 3.67 (m, 2H, H9), 2.28 (t, 2H, J=7.4 Hz, H12), 1.55 (m, 2H, H13), 1.28 (m, 8H, H14-H17), 0.87 (m, 3H, H18); 6C (126 MHz, DMSO-d6, 373K) 191.3, 172.0, 170.8, 168.3, 135.3, 132.8, 131.4, 129.2, 129.0, 127.8, 127.4, 126.0, 66.3, 62.9, 60.7, 47.2, 33.0, 30.4, 27.8, 27.6, 23.8, 21.3, 13.0; HRMS (ESI) m/z found 546.1499 [M+H]⁺, required 546.1491 [C27H33NO679Br]⁺.

### Example 3: Synthesis of an Enzyme Cleavable Linker of Formula (I)

FIG. 8 shows a synthetic pathway for an enzyme cleavable linker of formula (I) comprising the synthetic procedures outlined below.

### allyl 2-((2-(octanoyloxy)ethyl)amino)-2-methylpropanoate

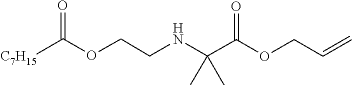

Allyl 2-amino-2-methylpropanoate (0.628 mmol) and 2-oxoethyl octanoate (0.515 mmol) were dissolved in DCM (5 mL) was stirred for 1 hour before NaBH(OAc)₃ (0.802 mmol) were added and the resulting suspension stirred for 17 h. The reaction was quenched with sat. NaHCO₃(aq.) (150 mL) and extracted with CH₂Cl₂ (3×15 mL). The combined organic layers were dried (MgSO₄), concentrated in vacuo and purified by flash column chromatography (20-50% EtOAc in heptane) to yield desired amine (0.271 mmol, 47%) as a colourless oil.

¹H NMR (400 MHz, DMSO-d₆): δ=5.93 (ddt, J=17.3, 10.8, 5.3 Hz, 1H), 5.31 (dq, J=17.1, 1.8 Hz, 1H), 5.21 (dq, J=10.5, 1.5 Hz, 1H), 4.57 (dt, J=5.4, 1.6 Hz, 2H), 4.03 (t, J=5.8 Hz, 2H), 2.75 (t, J=5.6 Hz, 2H), 2.28 (t, J=7.3 Hz, 2H), 1.55 (s, 6H), 1.51 (p, J=7.2 Hz, 2H), 1.32-1.17 (m, 8H), 0.90-0.77 (m, 3H); UPLC-MS (ESI): m/z found 314.3 [M+H]⁺, required 314.2 [C₁₇H₃₂NO₄]⁺.

### 2-(N-(1-(allyloxy)-2-methyl-1-oxopropan-2-yl)benzamido)ethyl octanoate

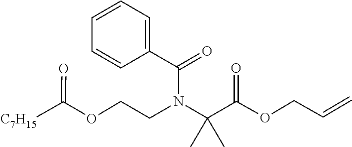

Benzoyl chloride (0.706 mmol) was added to a solution of triethylamine (98.4 μL, 0.706 mmol) and 2-((1-(allyloxy)-2-methyl-1-oxopropan-2-yl)amino)ethyl octanoate (0.543 mmol) in CH₂Cl₂ (1 mL) at 0° C. The solution was warmed up to rt and stirred for 2 h before being quenched with water (10 mL) and extracted with CH₂Cl₂ (20 mL). The organic layer was washed with 1 M HCl (aq.) (10 mL), sat. NaHCO₃(aq.) (10 mL) and brine (10 mL). The organic layer was dried (MgSO₄), concentrated in vacuo and purified by flash column chromatography (25% EtOAc in PE) to yield the desired amide (0.261 mmol, 48%) as a colourless oil.

1H-NMR (500 MHz, DMSO-d₆): δ=7.47 (m, 3H), 7.33 (m, 2H), 5.92 (m, 1H), 5.31 (d, 1H, J=17.4 Hz), 5.23 (d, 1H, J=10.4 Hz), 4.62 (d, 2H, J=4.9 Hz), 4.20 (m, 2H), 3.62 (m, 2H), 2.25 (t, 2H, J=7.3 Hz), 1.71 (s, 6H), 1.55 (m, 2H), 1.26 (m, 8H), 0.87 (m, 3H); UPLC-MS (ESI): m/z found 412.2105 [M+Na]+, required 412.2100 [C22H31NO5Na]⁺.

### 2-methyl-2-(N-(2-(octanoyloxy)ethyl)benzamido)propanoic acid

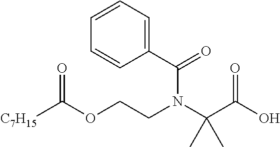

Tetrakis(triphenylphosphine)palladium(0) (19.6 mg, 16.9 μmol) and allyl ester 2-(N-(1-(allyloxy)-2-methyl-1-oxopropan-2-yl)benzamido)ethyl octanoate (0.250 mmol) were dissolved in THF (20 mL) and stirred at rt for 5 min. Morpholine (216 μL, 2.50 mmol) was added dropwise to the yellow solution and stirred for 4 h at rt. The resulting green solution was concentrated in vacuo and diluted with ether (10 mL) before being washed with 1 M HCl (aq.) (3×10 mL). The organic layer was filtered through a bed of celite washing thoroughly with ether, dried (MgSO4), concentrated in vacuo to give the desired acid as a pale yellow oil. Used without further purification in the next step. UPLC-MS (ESI) m/z found 400.2105 [M+Na]+, required 400.21 [C21H31NO5Na+].

### 2-(N-(1-(2-(4-bromophenyl)-2-oxoethoxy)-2-methyl-1-oxopropan-2-yl)benzamido)ethyl octanoate

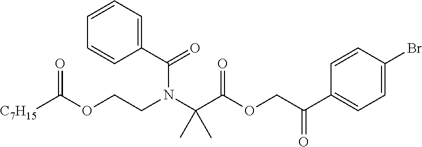

EDC (0.374 mmol) was added to a solution of acid N-benzoyl-N-(2-(octanoyloxy)ethyl)glycine (0.250 mmol), DMAP (0.374 mmol) and 1-(4-bromophenyl)-2-hydroxyethan-1-one (0.374 mmol) at 0° C. The suspension was warmed up to rt and stirred for 3 h before being diluted with CH₂Cl₂ (10 mL) and washed with sat. NaHCO₃(aq.) (10 mL) and 1 M HCl (aq.) (10 mL). The organic layer was dried (MgSO4), concentrated in vacuo and purified by flash column chromatography (30% EtOAc in heptane) to yield 2-(N-(1-(2-(4-bromophenyl)-2-(λ3-oxidaneylidene)ethoxy)-2-methyl-1-oxopropan-2-yl)benzamido)ethyl octanoate (0.120 mmol, 48%) as a colourless oil.

1H-NMR (500 MHz, DMSO-d₆): δ=7.88 (d, 2H, J=8.5 Hz), 7.75 (d, 2H, J=8.5 Hz), 7.46 (m, 3H), 7.38 (m, 2H), 5.46 (s, 2H), 4.21 (m, 2H), 3.67 (m, 2H), 2.28 (t, 2H, J=7.4 Hz), 1,28 (s, 6H), 1.55 (m, 2H), 1.28 (m, 8H), 0.87 (m, 3H). HRMS (ESI): m/z found 574.19 [M+H]+, required 574.18 [C29H37NO6Br]+.

### Example 4: Synthesis of an Enzyme Cleavable Linker of Formula (I)

FIG. 8 shows a synthetic pathway for an enzyme cleavable linker of formula (I) comprising the synthetic procedures outlined below.

### allyl 1-((2-(octanoyloxy)ethyl)amino)cyclopropane-1-carboxylate

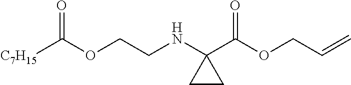

Allyl 1-aminocyclopropane-1-carboxylate (0.628 mmol) and 2-oxoethyl octanoate (0.515 mmol) were dissolved in DCM (5 mL) was stirred for 1 hour before NaBH(OAc)₃ (0.802 mmol) were added and the resulting suspension stirred for 17 h. The reaction was quenched with sat. NaHCO₃(aq.) (150 mL) and extracted with CH₂Cl₂ (3×15 mL). The combined organic layers were dried (MgSO₄), concentrated in vacuo and purified by flash column chromatography (20-50% EtOAc in heptane) to yield desired amine (0.273 mmol, 53%) as a colourless oil.

¹H NMR (400 MHz, DMSO-d₆) δ=5.92 (ddt, J=17.3, 10.7, 5.4 Hz, 1H), 5.31 (dq, J=17.2, 1.7 Hz, 1H), 5.21 (dq, J=10.5, 1.4 Hz, 1H), 4.57 (dt, J=5.5, 1.5 Hz, 2H), 4.03 (t, J=5.7 Hz, 2H), 2.75 (t, J=5.7 Hz, 2H), 2.28 (t, J=7.4 Hz, 2H), 1.51 (p, J=7.2 Hz, 2H), 1.32-1.17 (m, 10H), 0.90-0.77 (m, 5H); UPLC-MS (ESI): m/z found 312.4 [M+H]⁺, required 312.2 [C₁₇H₃₀NO₄]⁺.

### allyl 1-(N-(2-(octanoyloxy)ethyl)benzamido)cyclopropane-1-carboxylate

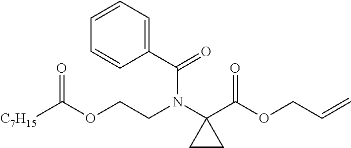

Benzoyl chloride (0.355 mmol) was added to a solution of triethylamine (98.4 μL, 0.353 mmol) and 2-((1-(allyloxy)-2-methyl-1-oxopropan-2-yl)amino)ethyl octanoate (0.273 mmol) in CH₂Cl₂ (1 mL) at 0° C. The solution was warmed up to rt and stirred for 2 h before being quenched with water (10 mL) and extracted with CH₂Cl₂ (20 mL). The organic layer was washed with 1 M HCl (aq.) (10 mL), sat. NaHCO₃(aq.) (10 mL) and brine (10 mL). The organic layer was dried (MgSO₄), concentrated in vacuo and purified by flash column chromatography (25% EtOAc in PE) to yield the desired amide (0.137 mmol, 50%) as a colourless oil.

δH (500 MHz, DMSO-d₆, 373K): 7.44 (m, 3H), 7.33 (m, 2H), 5.91 (m, 1H), 5.31 (d, 1H, J=17.5 Hz), 5.22 (d, 1H, J=10.5 Hz), 4.62 (d, 2H, J=5.0 Hz), 4.19 (m, 2H), 3.64 (m, 2H), 2.27 (t, 2H, J=7.3 Hz), 1.54 (m, 2H), 1.27 (m, 10H), 0.89 (m, 5H); UPLC-MS (ESI) m/z found 438.25 [M+Na]+, required 438.23 [C24H33NO5Na+].

### 1-(N-(2-(octanoyloxy)ethyl)benzamido)cyclopropane-1-carboxylic acid

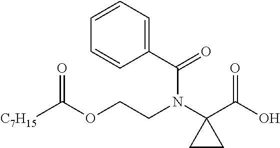

Tetrakis(triphenylphosphine)palladium(O) (10.7 mg, 9.3 μmol) and allyl ester 2-(N-(1-(allyloxy)-2-methyl-1-oxopropan-2-yl)benzamido)ethyl octanoate (0.137 mmol) were dissolved in THF (15 mL) and stirred at rt for 5 min. Morpholine (118 μL, 1.37 mmol) was added dropwise to the yellow solution and stirred for 4 h at rt. The resulting green solution was concentrated in vacuo and diluted with ether (10 mL) before being washed with 1 M HCl (aq.) (3×10 mL). The organic layer was filtered through a bed of celite washing thoroughly with ether, dried (MgSO4), concentrated in vacuo to give the desired acid as a pale yellow oil. Used without further purification in the next step. UPLC-MS (ESI) m/z found 398.21, [M+Na]+, required 398.19 [C21H29NO5Na]⁺.

### 2-(4-bromophenyl)-2-oxoethyl 1-(N-(2-(octanoyloxy)ethyl)benzamido)cyclopropane-1-carboxylate

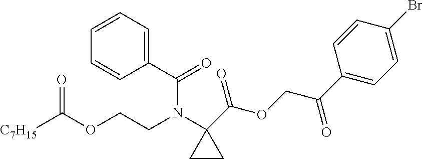

EDC (0.374 mmol) was added to a solution of acid allyl 1-((2-(octanoyloxy)ethyl)amino)cyclopropane-1-carboxylate (0.250 mmol), DMAP (0.374 mmol) and 1-(4-bromophenyl)-2-hydroxyethan-1-one (0.374 mmol) at 0° C. The suspension was warmed up to rt and stirred for 3 h before being diluted with CH2Cl2 (10 mL) and washed with sat. NaHCO3(aq.) (10 mL) and 1 M HCl (aq.) (10 mL). The organic layer was dried (MgSO4), concentrated in vacuo and purified by flash column chromatography (30% EtOAc in heptane) to yield 2-(4-bromophenyl)-2-oxoethyl 1-(N-(2-(octanoyloxy)ethyl)benzamido)cyclopropane-1-carboxylate (0.120 mmol, 48%) as a colourless oil.

1H-NMR (500 MHz, DMSO-d₆): δ=7.90 (d, 2H, J=8.6 Hz), 7.74 (d, 2H, J=8.6 Hz), 7.45 (m, 3H), 7.38 (m, 2H), 5.46 (s, 2H), 4.21 (m, 2H), 3.67 (m, 2H), 2.28 (t, 2H, J=7.3 Hz), 1.55 (m, 2H), 1.28 (m, 8H), 1.18-0.87 (m, 7H). UPLC-MS (ESI) m/z found 572.18 [M+H]⁺, required 572.16 [C29H35NO6Br]⁺.

### Example 5: Synthesis of an Enzyme Cleavable Linker of Formula (I)

FIG. 8 shows a synthetic pathway for an enzyme cleavable linker of formula (I) comprising the synthetic procedures outlined below.

### 2-((4-((Allyloxy)carbonyl)heptan-4-yl)amino)ethyl octanoate

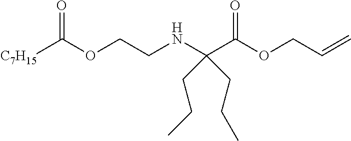

Allyl 2-amino-2-propylpentanoate (0.628 mmol) and 2-oxoethyl octanoate (0.515 mmol) were dissolved in DCM (5 mL) was stirred for 1 hour before NaBH(OAc)₃ (0.802 mmol) were added and the resulting suspension stirred for 17 h. The reaction was quenched with sat. NaHCO₃(aq.) (150 mL) and extracted with CH₂Cl₂ (3×15 mL). The combined organic layers were dried (MgSO₄), concentrated in vacuo and purified by flash column chromatography (20-50% EtOAc in heptane) to yield desired amine (0.144 mmol, 23%) as a colourless oil.

¹H NMR (400 MHz, DMSO-d₆) δ=5.92 (ddt, J=17.3, 10.7, 5.4 Hz, 1H), 5.31 (dq, J=17.2, 1.7 Hz, 1H), 5.21 (dq, J=10.5, 1.4 Hz, 1H), 4.57 (dt, J=5.5, 1.5 Hz, 2H), 4.03 (t, J=5.7 Hz, 2H), 3.39 (s, 2H), 2.75 (t, J=5.7 Hz, 2H), 2.28 (t, J=7.4 Hz, 2H), 1.53 (m, 6H), 1.32-1.17 (m, 12H), 0.90-0.77 (m, 9H); UPLC-MS (ESI): m/z found 370.4 [M+H]⁺, required 370.3 [C₂₁H₄₀NO₄]⁺.

### 2-(N-(4-((Allyloxy)carbonyl)heptan-4-yl)benzamido)ethyl octanoate

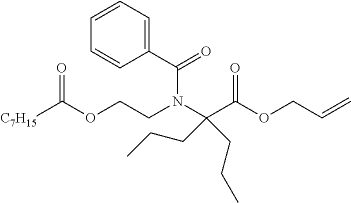

Benzoyl chloride (0.188 mmol) was added to a solution of triethylamine (52.2 μL, 0.187 mmol) and 2-((4-((allyloxy)carbonyl)heptan-4-yl)amino)ethyl octanoate (0.144 mmol) in CH₂Cl₂ (1 mL) at 0° C. The solution was warmed up to rt and stirred for 2 h before being quenched with water (10 mL) and extracted with CH₂Cl₂ (20 mL). The organic layer was washed with 1 M HCl (aq.) (10 mL), sat. NaHCO₃(aq.) (10 mL) and brine (10 mL). The organic layer was dried (MgSO₄), concentrated in vacuo and purified by flash column chromatography (40% EtOAc in PE) to yield the desired amide (0.089 mmol, 62%) as a colourless oil.

δH (500 MHz, DMSO-d₆): 7.45 (m, 3H), 7.35 (m, 2H), 5.92 (m, 1H), 5.31 (d, 1H, J=17.6 Hz), 5.23 (d, 1H, J=10.6 Hz), 4.62 (d, 2H, J=5.0 Hz), 4.20 (m, 2H), 3.64 (m, 2H), 2.27 (t, 2H, J=7.3 Hz), 1.67 (m, 4H), 1.54 (m, 2H, H15), 1.27 (m, 12H), 0.88 (m, 9H, H20); UPLC-MS (ESI) m/z found 496.33 [M+Na]+, required 496.30 [C28H43NO5Na]+.

### 2-(N-(2-(octanoyloxy)ethyl)benzamido)-2-propylpentanoic acid

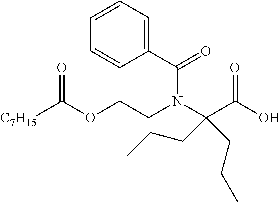

Tetrakis(triphenylphosphine)palladium(O) (7.0 mg, 6.1 μmol) and allyl ester 2-(N-(4-((allyloxy)carbonyl)heptan-4-yl)benzamido)ethyl octanoate (0.089 mmol) were dissolved in THF (10 mL) and stirred at rt for 5 min. Morpholine (77 μL, 0.89 mmol) was added dropwise to the yellow solution and stirred for 4 h at rt. The resulting green solution was concentrated in vacuo and diluted with ether (10 mL) before being washed with 1 M HCl (aq.) (3×10 mL). The organic layer was filtered through a bed of celite washing thoroughly with ether, dried (MgSO₄), concentrated in vacuo to give the desired acid as a pale yellow oil. Used without further purification in the next step. UPLC-MS (ESI) m/z found 352.23 [M+Na]⁺, required 362.25 [C18H35NO4Na]⁺.

### 2-(N-(4-((2-(4-bromophenyl)-2-oxoethoxy)carbonyl)heptan-4-yl)benzamido)ethyl octanoate

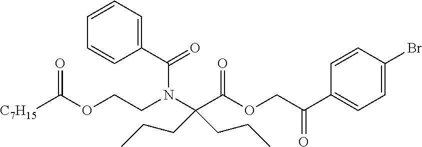

EDC (0.137 mmol) was added to a solution of acid 2-(N-(2-(octanoyloxy)ethyl)benzamido)-2-propylpentanoic acid (0.089 mmol), DMAP (0.133 mmol) and 1-(4-bromophenyl)-2-hydroxyethan-1-one (0.133 mmol) at 0° C. The suspension was warmed up to rt and stirred for 3 h before being diluted with CH₂Cl₂ (10 mL) and washed with sat. NaHCO3(aq.) (10 mL) and 1 M HCl (aq.) (10 mL). The organic layer was dried (MgSO4), concentrated in vacuo and purified by flash column chromatography (50% EtOAc in heptane) to yield 2-(N-(4-((2-(4-bromophenyl)-2-oxoethoxy)carbonyl)heptan-4-yl)benzamido)ethyl octanoate (0.034 mmol, 38%) as a colourless oil.

¹H-NMR (500 MHz, DMSO-d₆): δ=7.88 (d, 2H, J=8.5 Hz), 7.75 (d, 2H, J=8.5 Hz), 7.46 (m, 3H), 7.38 (m, 2H), 5.46 (s, 2H), 4.21 (m, 2H), 3.67 (m, 2H), 2.28 (t, 2H, J=7.4 Hz), 1,86 (m, 4H), 1.55 (m, 2H, H13), 1.28 (m, 12H), 0.87 (m, 9H). UPLC-MS (ESI) m/z found 652.24 [M+Na]⁺, required 652.22 [C33H44BrNO6Na]⁺.

### Example 6: Synthesis of an Enzyme Cleavable Linker of Formula (I)

FIG. 8 shows a synthetic pathway for an enzyme cleavable linker of formula (I) comprising the synthetic procedures outlined below.

### 2-((2-(allyloxy)-2-oxo-1,1-diphenylethyl)amino)ethyl octanoate

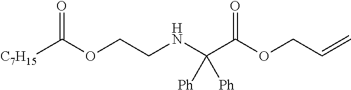

Allyl 2-amino-2,2-diphenylacetate (0.628 mmol) and 2-oxoethyl octanoate (0.515 mmol) were dissolved in DCM (5 mL) was stirred for 1 hour before NaBH(OAc)₃ (0.802 mmol) were added and the resulting suspension stirred for 17 h. The reaction was quenched with sat. NaHCO₃(aq.) (150 mL) and extracted with CH₂Cl₂ (3×15 mL). The combined organic layers were dried (MgSO₄), concentrated in vacuo and purified by flash column chromatography (20-50% EtOAc in heptane) to yield desired amine (0.200 mmol, 32%) as a colourless oil.

¹H NMR (400 MHz, DMSO-d₆): δ=7.37-7.29 (m, 10H), 5.93 (ddt, J=17.3, 10.7, 5.4 Hz), 5.30 (dq, J=17.1, 1.8 Hz, 1H), 5.21 (dq, J=10.5, 1.4 Hz, 1H), 4.57 (dt, J=5.5, 1.5 Hz, 2H), 4.03 (t, J=5.7 Hz, 2H), 3.39 (s, 2H), 2.75 (t, J=5.7 Hz, 2H), 2.28 (t, J=7.4 Hz, 2H), 1.51 (p, J=7.2 Hz, 2H), 1.32-1.17 (m, 8H), 0.90-0.77 (m, 3H); UPLC-MS (ESI) m/z required 438.3 [M+H]⁺, required 438.3 [C₂₇H₃₆NO₄]⁺.

### 2-(N-(2-(allyloxy)-2-oxo-1,1-diphenylethyl)benzamido)ethyl octanoate

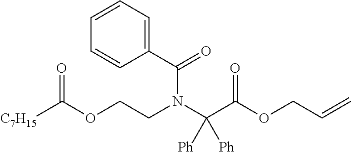

Benzoyl chloride (0.261 mmol) was added to a solution of triethylamine (72.6 μL, 0.260 mmol) and 2-((2-(allyloxy)-2-oxo-1,1-diphenylethyl)amino)ethyl octanoate (0.200 mmol) in CH₂Cl₂ (1 mL) at 0° C. The solution was warmed up to rt and stirred for 2 h before being quenched with water (10 mL) and extracted with CH2Cl2 (20 mL). The organic layer was washed with 1 M HCl (aq.) (10 mL), sat. NaHCO3(aq.) (10 mL) and brine (10 mL). The organic layer was dried (MgSO4), concentrated in vacuo and purified by flash column chromatography (60% EtOAc in heptane) to yield the desired amide (0.118 mmol, 59%) as a colourless oil.

1H-NMR (500 MHz, DMSO-d₆): δ=7.45 (m, 3H), 7.36 (m, 2H), 7.35-7.28 (m, 10H), 5.92 (m, 1H), 5.31 (d, 1H, J=17.6 Hz), 5.23 (d, 1H, J=10.6 Hz), 4.62 (d, 2H, J=5.0 Hz), 4.20 (m, 2H), 3.64 (m, 2H), 2.27 (t, 2H, J=7.3 Hz), 1.54 (m, 2H), 1.28 (m, 8H), 0.88 (m, 3H); UPLC-MS (ESI) m/z found 564.25 [M+Na]⁺, required 564.27 [C34H39NO5Na]⁺.

### 2-(N-(2-(octanoyloxy)ethyl)benzamido)-2,2-diphenylacetic acid

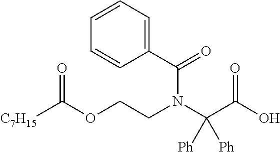

Tetrakis(triphenylphosphine)palladium(O) (7.0 mg, 6.1 μmol) and allyl ester 2-(N-(2-(allyloxy)-2-oxo-1,1-diphenylethyl)benzamido)ethyl octanoate (0.089 mmol) were dissolved in THF (10 mL) and stirred at rt for 5 min. Morpholine (77 μL, 0.89 mmol) was added dropwise to the yellow solution and stirred for 4 h at rt. The resulting solution was concentrated in vacuo and diluted with ether (10 mL) before being washed with 1 M HCl (aq.) (3×10 mL). The organic layer was filtered through a bed of celite washing thoroughly with ether, dried (MgSO4), concentrated in vacuo to give the desired acid as a pale yellow oil. Used without further purification in the next step. LCMS (ESI) m/z found 524.23 [M+Na]⁺, required 524.24 [C31H35NO5Na]⁺.

### 2-(N-(2-(2-(4-bromophenyl)-2-oxoethoxy)-2-oxo-1,1-diphenylethyl)benzamido)ethyl octanoate

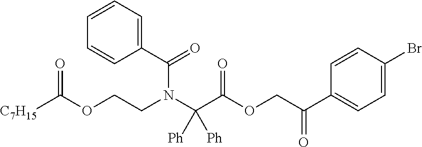

EDC (0.137 mmol) was added to a solution of acid 22-(N-(2-(octanoyloxy)ethyl)benzamido)-2,2-diphenylacetic acid (0.089 mmol), DMAP (0.133 mmol) and 1-(4-bromophenyl)-2-hydroxyethan-1-one (0.133 mmol) at 0° C. The suspension was warmed up to rt and stirred for 3 h before being diluted with CH2Cl2 (10 mL) and washed with sat. NaHCO3(aq.) (10 mL) and 1 M HCl (aq.) (10 mL). The organic layer was dried (MgSO₄), concentrated in vacuo and purified by flash column chromatography (50% EtOAc in heptane) to yield 2-(N-(2-(2-(4-bromophenyl)-2-oxoethoxy)-2-oxo-1,1-diphenylethyl)benzamido)ethyl octanoate (0.034 mmol, 38%) as a colourless oil.

1H-NMR (500 MHz, DMSO-d₆): δ=7.87 (d, 2H, J=8.5 Hz, H3), 7.76 (d, 2H, J=8.5 Hz, H2), 7.48 (m, 3H, H21 and H23), 7.36 (m, 2H, H22), 7.35-7.7.26 (m, 10H, 5.46 (s, 2H), 4.21 (m, 2H), 3.67 (m, 2H), 2.28 (t, 2H, J=7.5 Hz), 1.55 (m, 2H), 1.26 (m, 8H), 0.85 (m, 3H). UPLC-MS (ESI): m/z found 720.22 [M+Na]⁺, required 720.19 [C39H40BrNO6Na]⁺.

### Example 7: Synthesis of an Enzyme Cleavable Linker of Formula (I)

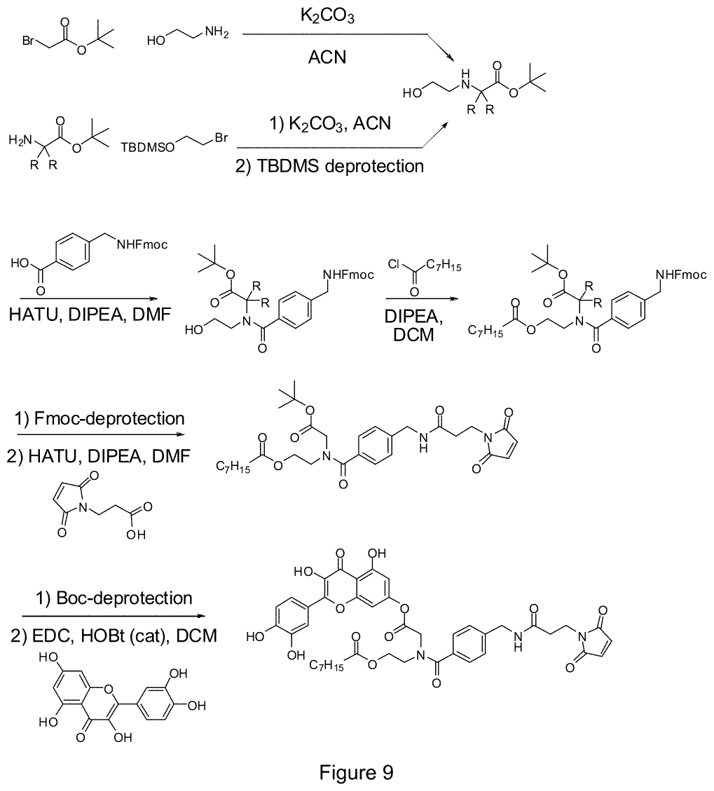

FIG. 9 shows a synthetic pathway for an enzyme cleavable linker of formula (I) comprising the synthetic procedures outlined below.

### tert-butyl (2-hydroxyethyl)glycinate

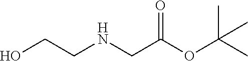

To a 0° C. solution of 2-aminoethanol (2.00 g, 32.8 mmol) and K₂CO₃ (22.60 g, 164 mmol) in 80 mL acetonitrile was added a solution of tert-butyl 2-bromoacetate (32.8 mmol) in 2 mL of acetonitrile over a 1 h period. The solution was stirred for 1 h at 0° C. and filtered. The filtrate was concentrated in vacuo to give a residue which was subjected to flash column chromatography (0% to 10% MeOH in EtOAc) to yield the desired product in 44%.

R_{f}=0.16 (5% MeOH in EtOAc); ¹H NMR (400 MHz, Chloroform-d) δ=3.62-3.56 (m, 2H), 3.29 (s, 2H), 2.78-2.69 (m, 2H), 1.43 (d, J=3.0 Hz, 9H); ¹³C NMR (101 MHz, Chloroform-d) δ=172.12, 81.49, 60.99, 51.29, 51.16, 28.17; UPLC-MS (ESI) m/z found 176.15 [M+H]⁺, required 176.13 [C8H18NO3]⁺.

### tert-butyl N-(4-(((((9H-fluoren-9-yl)methoxy)carbonyl)amino)methyl)benzoyl)-N-(2-hydroxyethyl)glycinate

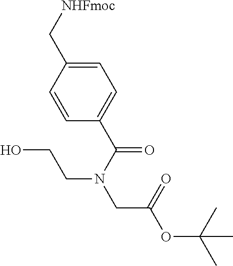

A suspension of 4-(((((9H-fluoren-9-yl)methoxy)carbonyl)amino)methyl)benzoic acid (5.97 g, 16.0 mmol), DIPEA (14.0 mL, 79.9 mmol) and tert-butyl (2-hydroxyethyl)glycinate (2.8 g, 16.0 mmol) in DMF (40 mL) was stirred for 5 min before HATU (5.85 g, 16.0 mmol) was added at rt. After 12 h the reaction was diluted with 75 mL EtOAc and washed with brine (4×75 mL), NH4Cl (75 mL) and NaHCO₃(75 mL). The organic phases were dried (MgSO₄), concentrated in vacuo and purified by flash column chromatography (50% to 100% EtOAc in heptane) to yield the desired product (77%) as a white solid.

R_{f}=0.34 (EtOAc); ¹H NMR (400 MHz, Chloroform-d) δ=7.76 (d, J=7.6 Hz, 2H), 7.60 (d, J=7.5 Hz, 2H), 7.50 (d, J=7.8 Hz, 2H), 7.40 (t, J=7.5 Hz, 2H), 7.35-7.25 (m, 4H), 5.18 (s, 1H), 4.48 (d, J=6.8 Hz, 2H), 4.39 (d, J=5.9 Hz, 2H), 4.23 (t, J=6.7 Hz, 1H), 4.10 (s, 2H), 3.61 (t, J=5.0 Hz, 2H), 3.48 (t, J=4.9 Hz, 2H), 1.48 (d, J=38.6 Hz, 9H); ¹³C NMR (101 MHz, Chloroform-d) δ=172.57, 170.86, 156.61, 143.98, 141.47, 140.13, 134.85, 128.07, 127.84, 127.49, 127.20, 125.13, 120.13, 83.19, 66.84, 59.40, 53.31, 49.16, 47.43, 44.80, 28.15; UPLC-MS (ESI) m/z found 552.9 [M+Na]⁺, required 553.2 [C₃₁H₃₄N₂NaO_{6]}⁺.

### 2-(4-(((((9H-fluoren-9-yl)methoxy)carbonyl)amino)methyl)-N-(2-(tert-butoxy)-2-oxoethyl)benzamido)ethyl octanoate

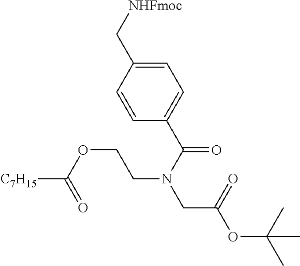

Octanoyl chloride (2.19 mL, 12.9 mmol) was added dropwise to a solution of tert-butyl N-(4-(((((9H-fluoren-9-yl)methoxy)carbonyl)amino)methyl)benzoyl)-N-(2-hydroxyethyl)glycinate (1.37 g, 2.57 mmol) and DIPEA (4.48 mL, 25.7 mmol) in CH₂Cl₂ (50 mL) at rt. After 14 h the reaction was quenched with H₂O (100 mL) and extracted with CH₂Cl₂ (4×60 mL). The combined organic layers were washed with H₂O (150 mL), brine (3×150 mL), NH₄Cl (150 mL), NaHCO₃ (150 mL) and brine (150 mL), dried (MgSO₄) and concentrated in vacuo. The crude product was purified by flash column chromatography (100% heptane to 100% EtOAc) to yield the desired product (1.12 mg, 1.70 mmol, 66%) as a yellow oil.

R_{f}=0.73 (EtOAc); ¹H NMR (400 MHz, Chloroform-d) δ=7.77 (d, J=7.5 Hz, 2H), 7.60 (d, J=7.5 Hz, 2H), 7.46-7.26 (m, 8H), 5.10 (d, J=6.2 Hz, 1H), 4.49 (d, J=6.8 Hz, 2H), 4.40 (d, J=5.3 Hz, 2H), 4.36 (s, OH), 4.23 (t, J=6.8 Hz, 1H), 4.19 (s, 1H), 4.11 (dd, J=8.6, 5.7 Hz, 1H), 3.93 (s, 1H), 3.79 (d, J=5.7 Hz, 1H), 3.58 (t, J=5.7 Hz, 1H), 2.31 (dt, J=13.1, 7.5 Hz, 2H), 1.61 (dt, J=13.7, 6.8 Hz, 2H), 1.47 (d, J=29.9 Hz, 9H), 1.36-1.16 (m, 8H), 0.94-0.77 (m, 3H); ¹³C NMR (101 MHz, Chloroform-d) δ=172.21, 168.56, 168.15, 156.61, 143.97, 141.50, 140.41, 135.11, 127.87, 127.63, 127.38, 127.20, 125.12, 120.15, 82.18, 66.88, 60.54, 53.02, 48.18, 47.44, 44.81, 34.25, 31.78, 29.24, 29.06, 28.25, 28.14, 24.93, 22.73, 21.21, 14.35, 14.21; UPLC-MS (ESI) m/z found 657.1 [M+H]⁺, required 657.4 [C₃₉H₄₉N₂O₇]⁺.

### 2-(N-(2-(tert-Butoxy)-2-oxoethyl)-4-((3-(2,5-dioxo-2,5-dihydro-1H-pyrrol-1-yl)propanamido)methyl)benzamido)ethyl octanoate

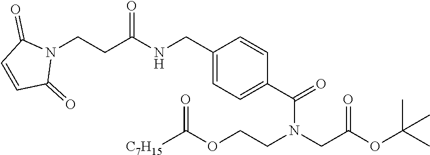

2-(4-(((((9H-Fluoren-9-yl)methoxy)carbonyl)amino)methyl)-N-(2-(tert-butoxy)-2-oxoethyl)benzamido)ethyl octanoate (390 mg, 0.59 mmol) was dissolved in diethylamine and DCM (1:1, 7 mL) and stirred at rt until complete Fmoc deprotection (4 h). The reaction mixture was concentrated in vacuo and used directly in next step. The crude tert-butyl N-(4-(aminomethyl)benzoyl)-N-(2-hydroxyethyl)glycinate was dissolved in anhydrous DMF (10 mL) and stirred under nitrogen atmosphere. 3-(2-hydroxy-5-oxo-2,5-dihydro-1H-pyrrol-1-yl)propanoic acid (100 mg, 0.59 mmol), HATU (271 mg, 0.71 mmol) and anhydrous DIPEA (517 μL, 2.97 mmol) were added and the solution was stirred at rt for 3 h. The reaction was diluted with EtOAc (50 mL) and washed with water (60 mL), brine (4×60 mL), NH₄Cl (60 mL), NaHCO₃(60 mL) and brine (60 mL). The organic phases were dried over MgSO₄, concentrated in vacuo and purified by flash column chromatography (heptane EtOAc+5% MeOH) to yield the title compound as a yellow oil (152 mg, 44%).

R_{f}=0.36 (EtOAc); ¹H NMR (400 MHz, DMSO-d₆) δ 8.50 (t, J=5.6 Hz, 1H), 7.35-7.22 (m, 4H), 7.01 (s, 2H), 4.23 (dt, J=12.1, 5.9 Hz, 4H), 4.08 (s, 2H), 3.65 (tt, J=7.4, 4.5 Hz, 4H), 2.43 (t, J=7.3 Hz, 2H), 2.29-2.25 (m, 2H), 1.43 (s, 9H), 1.30-1.19 (m, 10H), 0.85 (t, J=6.5 Hz, 3H); UPLC-MS (ESI) m/z found 686.8 [M+H]⁺, calc. 686.7 [C₃₁H₄₄N₃O₈]⁺.

### 2-(N-(2-((2-(3,4-dihydroxyphenyl)-3,5-dihydroxy-4-oxo-4H-chromen-7-yl)oxy)-2-oxoethyl)-4-((3-(2,5-dioxo-2,5-dihydro-1H-pyrrol-1-yl)propanamido)methyl)benzamido)ethyl octanoate

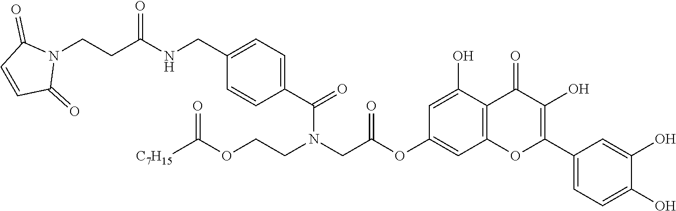

2-(N-(2-(tert-Butoxy)-2-oxoethyl)-4-((3-(2,5-dioxo-2,5-dihydro-1H-pyrrol-1-yl)propanamido)methyl)benzamido)ethyl octanoate (150 mg, 0.26 mmol) was dissolved in TFA/DCM (4:6, 5 mL) and stirred at rt until complete tert-butyl deprotection (2 h). The reaction mixture was concentrated in vacuo and co-evaporated with THF (×7). EDC (226 μL, 1.27 mmol) and HOBt (62 mg, 0.51 mmol) were added to a solution of the crude N-(4-((3-(2,5-dioxo-2,5-dihydro-1H-pyrrol-1-yl)propanamido)methyl)benzoyl)-N-(2-(octanoyloxy)ethyl)glycine in anhydrous DCM (3 mL). Additionally, DIPEA (111 μL, 0.63 mmol) was added to achieve a basicity of pH 7-8. A solution of quercitin (77 mg, 0.25 mmol) in anhydrous DMF (1 mL) was added at rt, and the solution was stirred under nitrogen atmosphere overnight. The crude reaction mixture was purified directly using preparative HPLC (linear gradient) and lyophilized yielding the title compound as a yellow solid (64 mg, 31%).

¹H NMR (400 MHz, DMSO-d₆) δ 12.38 (d, J=23.4 Hz, 1H), 10.90-10.77 (m, 1H), 10.50 (d, J=54.3 Hz, 1H), 9.67 (d, J=48.3 Hz, 1H), 8.51 (t, J=5.9 Hz, 1H), 7.97 (d, J=8.6, 2.3 Hz, 1H), 7.42-7.26 (m, 5H), 7.10 (d, J=8.7 Hz, 1H), 7.00 (s, 2H), 6.44 (d, J=2.1 Hz, 1H), 6.19 (d, J=2.0 Hz, 1H), 4.56 (d, J=8.8 Hz, 1H), 4.47-4.39 (m, 1H), 4.36-4.20 (m, 3H), 4.19-4.10 (m, 1H), 3.86-3.74 (m, 1H), 3.69-3.58 (m, 3H), 2.43 (t, J=7.3 Hz, 2H), 2.34-2.18 (m, 2H), 1.58-1.38 (m, 2H), 1.22-1.13 (m, 8H), 0.87-0.79 (m, 3H); UPLC-MS (ESI) m/z found 814.8 [M+H]⁺, calc. 814.8 [C₄₂H₄₄N₃O₁₄]⁺.

### Example 8: Synthesis of an Enzyme Cleavable Linker of Formula (I)

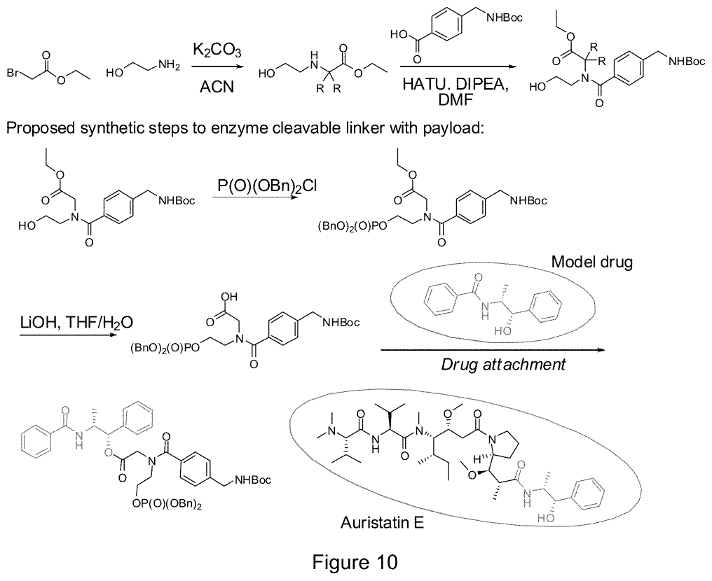

FIG. 10 shows a synthetic pathway for an enzyme cleavable linker of formula (I) comprising the synthetic procedures outlined below.

### Ethyl (2-hydroxyethyl)glycinate

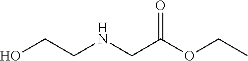

To a 0° C. solution of 2-aminoethanol (32.8 mmol) and K₂CO₃ (164 mmol) in 80 mL acetonitrile was added a solution of ethyl bromoacetate (32.8 mmol) in 2 mL of acetonitrile over a 1 h period. The solution was stirred for 1 h at 0° C. and filtered. The filtrate was concentrated in vacuo to give a residue which was subjected to flash column chromatography (0% to 10% MeOH in EtOAc) to yield the desired product in 41%.

R_{f}=0.16 (5% MeOH in EtOAc); ¹H NMR (400 MHz, Chloroform-d): δ=5.48 (bs, 1H), 4.71 (bs, 1H), 4.15 (quart, 2H), 3.58 (s, 2H), 3.51 (m, 2H), 2.74 (m, 2H), 1.31 (t, 2H); UPLC-MS (ESI) m/z found 148.13 [M+H]⁺, required 148.10 [C6H14NO_{3]}⁺.

### Ethyl N-(4-(((tert-butoxycarbonyl)amino)methyl)benzoyl)-N-(2-hydroxyethyl)glycinate

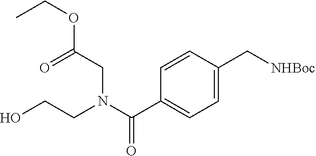 [SMILES: CCOC(=O)CN(CCO)C(=O)c1ccc(CNC(=O)OC(C)(C)C)cc1]

A suspension of 4-(((tert-butoxycarbonyl)amino)methyl)benzoic acid (16.0 mmol), DIPEA (14.0 mL, 79.9 mmol) and Ethyl (2-hydroxyethyl)glycinate (16.0 mmol) in DMF (40 mL) was stirred for 5 min before HATU (5.85 g, 16.0 mmol) was added at rt. After 12 h the reaction was diluted with 75 mL EtOAc and washed with brine (4×75 mL), NH₄Cl (75 mL) and NaHCO₃(75 mL). The organic phases were dried (MgSO₄), concentrated in vacuo and purified by flash column chromatography (50% to 100% EtOAc in heptane) to yield the desired product (39%) as a white oil.

R_{f}=0.34 (EtOAc); ¹H NMR (400 MHz, Chloroform-d) δ=7.48 (d, J=7.8 Hz, 2H), 7.31 (d, J=7.5 Hz, 3H), 4.97-4.82 (m, 1H), 4.30 (dt, J=21.3, 6.6 Hz, 4H), 4.19 (s, 2H), 3.62 (t, J=4.8 Hz, 2H), 3.52 (t, J=4.8 Hz, 2H), 1.46 (s, 9H), 1.33 (t, J=7.1 Hz, 3H); ¹³C NMR (101 MHz, Chloroform-d) δ=172.79, 171.73, 156.05, 140.84, 134.51, 127.96, 127.83, 127.46, 127.17, 79.88, 62.18, 59.56, 53.38, 48.49, 44.42, 28.53, 14.24; UPLC-MS (ESI) m/z found 403.21 [M+Na]⁺, required 403.18 [C₁₉H₂₈N₂NaO_{6]}⁺.

### Example 9: Synthesis of an Enzyme Cleavable Linker of Formula (II)

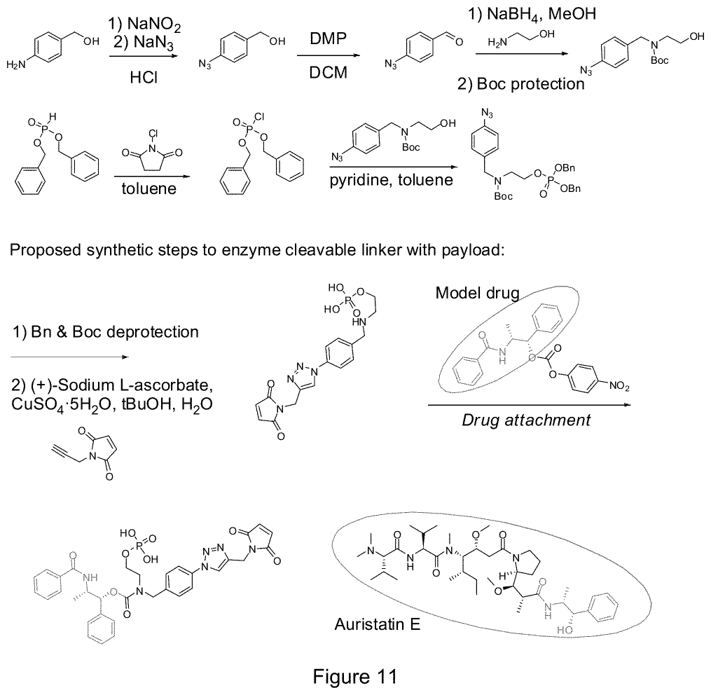

FIG. 11 shows a synthetic pathway for an enzyme cleavable linker of formula (II) comprising the synthetic procedures outlined below.

### (4-Azidophenyl)methanol

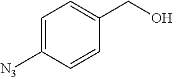 [SMILES: [N-]=[N+]=Nc1ccc(CO)cc1]

4-aminobenzylalcohol (1.0 g, 8.12 mmol, 1.0 eq.) was dissolved in 8.1 mL 6 M HCl and cooled in an ice bath to 0° C. A solution of NaNO₂ (0.84 g, 12.2 mmol, 1.5 eq.) in 21 mL H2O cooled to 0° C. was added dropwise. The yellow reaction mixture was stirred at 0° C. for 30 min. before dropwise addition of NaN3 (2.1 g, 32.5 mmol, 4.0 eq.) in 41 mL H2O keeping the temperature below 0° C. After 15 min. at 0° C., the reaction was allowed to slowly reach rt and was stirred for additionally 2 h. The reaction was quenched with 100 mL sat. aqueous NaHCO₃ and stirred for 30 min. before extraction with Et2O (3×100 mL). The organic phase was dried with MgSO₄ and concentrated in vacuo. The residue was dissolved in 3 mL EtOAc:Heptane (1:2) and purified through a plug of SiO2 using the same eluent system yielding the title compound as a light-yellow oil (3.60 g, 99%).

¹H-NMR (400 MHz, Chloroform-d) δ 7.28 (d, J=8.5 Hz, 2H), 6.97 (d, J=8.5 Hz, 2H), 4.56 (s, 2H), 2.88 (s, 1H). ¹³C-NMR (101 MHz, Chloroform-d) δ 139.27, 137.58, 128.52, 119.07, 64.44. UPLC-MS (ESI): m/z found 172.0 [M+Na]⁺, required 172.1 [C7H7N3NaO]⁺.

### 4-Azidobenzaldehyde

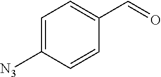 [SMILES: [N-]=[N+]=Nc1ccc(C=O)cc1]

(4-azidophenyl)methanol (3.60 g, 24.14 mmol, 1 eq.) was dissolved in 125 mL dry CH₂Cl₂ followed by addition of Dess—Martin periodinane (15.78 g, 37.21 mmol, 1.5 eq.) and the reaction mixture was stirred for 18 h at rt at which state oxidation was completed. The reaction mixture was diluted with EtOAc (100 mL) resulting in biproduct being precipitated. H2O was added resulting in more biproduct precipitating. The reaction mixture was filtrated and washed with CH₂Cl₂ followed by wash with sat. aqueous Na₂S₂O₃ (150 mL), sat. aqueous NaHCO₃ (150 mL), and brine (150 mL). The organic layer was dried with Na₂SO₄ and concentrated in vacuo. The crude product was purified by plug on SiO₂ using EtOAc:Heptane (1:4) yielding the title compound as a brown oil (3.29 g, 93%).

¹H-NMR (400 MHz, Chloroform-d) δ 9.92 (s, 1H), 7.86 (d, J=8.5 Hz, 2H), 7.14 (d, J=8.5 Hz, 2H). ¹³C-NMR (101 MHz, Chloroform-d) δ 190.62, 146.32, 133.31, 131.60, 119.54.

### tert-Butyl (4-azidobenzyl)(2-hydroxyethyl)carbamate

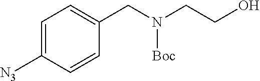 [SMILES: CC(C)(C)OC(=O)N(CCO)Cc1ccc(N=[N+]=[N-])cc1]

Ethanol amine (0.411 mL, 6.80 mmol, 1 eq.) was dissolved in dry MeOH (10 mL) under nitrogen atmosphere. MgSO₄ (1.64 g, 13.63 mmol, 2 eq.) was added followed by addition of 4-azidobenzaldehyde (1.00 g, 6.80 mmol, 1 eq.) dissolved in 3 mL dry MeOH, and the reaction mixture was stirred for 20 h. The reaction mixture was cooled to 0° C. and NaBH4 (0.257 g, 6.80 mmol, 1 eq.) was added portion wise. The reaction was stirred for 1.5 h before quenching with H2O. The reaction was extracted with EtOAc (5×100 mL) and the combined organic layers were washed with brine (1×500 mL). The organic layer was dried over MgSO₄ and concentrated in vacuo. The crude was dissolved in sat. aq. NH4Cl:dioxane (1:1) (20 mL) followed by addition of Boc anhydride (1.78 g, 8.16 mmol, 1.2 eq.) and the reaction mixture was stirred for 20 h. The reaction mixture was extracted with EtOAc (3×50 mL) and the combined organic phases were dried over Na2SO4 followed by concentration in vacuo. The crude product was purified by flash chromatography on SiO2 using EtOAc:Heptane (2:3) yielding the title compound as a brown oil (1.36 g, 68%).

(400 MHz, Chloroform-d) δ 7.23 (d, J=7.9 Hz, 2H), 6.99 (d, J=8.0 Hz, 2H), 4.46 (s, 2H), 3.77-3.62 (m, 2H), 3.50-3.23 (m, 2H), 2.81 (s, 1H), 1.47 (s, 9H). ¹³C-NMR (101 MHz, Chloroform-d) δ 157.19, 139.14, 135.16, 128.77, 119.24, 80.81, 62.00, 51.58, 49.83, 28.46. UPLC MS (ESI) m/z found 291.2 required 291.15 [C14H19N4O3]⁻.

### tert-Butyl (4-azidobenzyl)(2-((bis(benzyloxy)phosphoryl)oxy)ethyl)carbamate

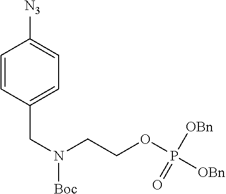 [SMILES: CC(C)(C)OC(=O)N(CCOP(=O)(OCc1ccccc1)OCc1ccccc1)Cc1ccc(N=[N+]=[N-])cc1]

Dibenzyl phosphorochloridate was prepared in situ by reacting N-chloro-succinimide (1.86 mg, 13.95 mmol, 1 eq.) and dibenzyl phosphonate (3.07 mL, 13.95 mmol, 1 eq.) in anhydrous toluene (25 mL) under inert atmosphere for 2 h at rt. The reaction mixture was filtered to remove succinimide. To a solution of tert-butyl (4-azidobenzyl)(2-hydroxyethyl)carbamate (1.36 g, 4.65 mmol, 1 eq.) in anhydrous pyridine (25 mL) at −40° C. under inert atmosphere was added dropwise a solution of dibenzyl phosphorochloridate in toluene. The reaction mixture was stirred for 2 h at −40° C. before it was placed in the freezer at −20° C. for 20 h. The reaction mixture was allowed to warm to rt and was subsequently quenched with H2O (20 mL) before concentration and co-evaporation with heptane in vacuo. The resulting crude residue was re-dissolved in EtOAC (200 mL) and the organic layer was washed with 1 M H2SO4 (2×75 mL) and sat. aq. NaHCO₃(2×75 mL). The organic layer was dried over Na2SO4 followed by concentration in vacuo. The crude product was purified by flash chromatography on SiO₂ using EtOAc:Heptane (2:3) yielding the title compound as a brown oil (1.61 g, 63%).

1H-NMR (400 MHz, DMSO-d₆) δ 7.42-7.31 (m, 10H), 7.22 (d, J=8.4 Hz, 2H), 7.06 (d, J=8.0 Hz, 2H), 5.01 (d, J=8.0 Hz, 4H), 4.34 (s, 2H), 4.08-3.95 (m, 2H), 3.32 (d, J=7.8 Hz, 2H), 1.35 (d, J=21.4 Hz, 9H). 13C-NMR (101 MHz, DMSO-d₆) δ 154.88, 138.16, 136.04, 135.97, 129.01, 128.72, 128.49, 128.41, 127.81, 119.16, 79.38, 68.61, 68.55, 64.89, 27.92. 31P-NMR (162 MHz, DMSO-d₆) δ −0.98 (d, J=10.5 Hz). UPLC MS (ESI): m/z found 553.6 [M+H]⁺, required 553.22 [C28H34N4O6P]⁺.

### Example 10: Synthesis of an enzyme cleavable linker of formula (II)

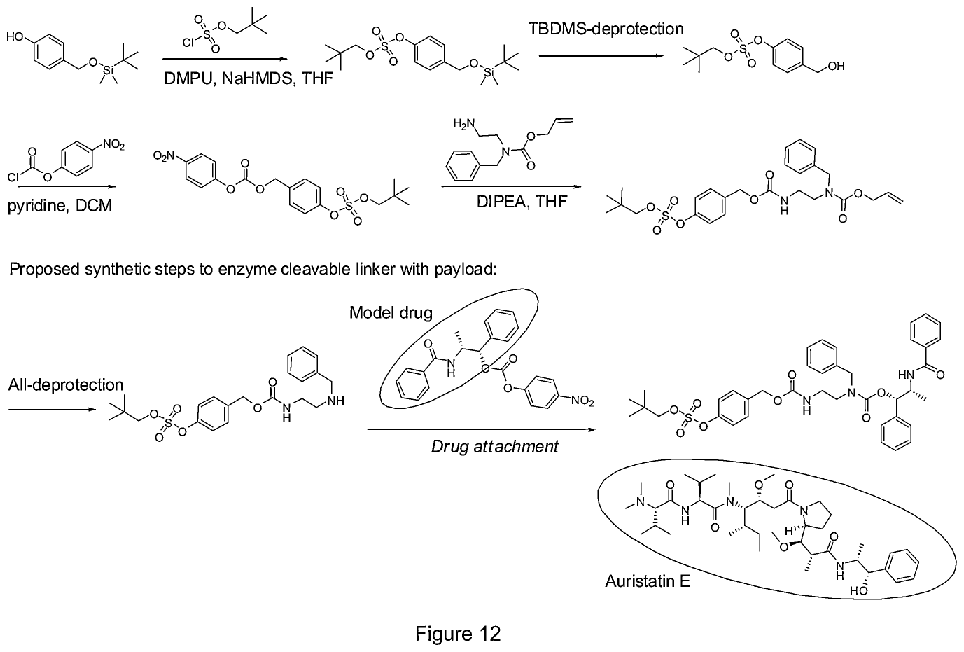

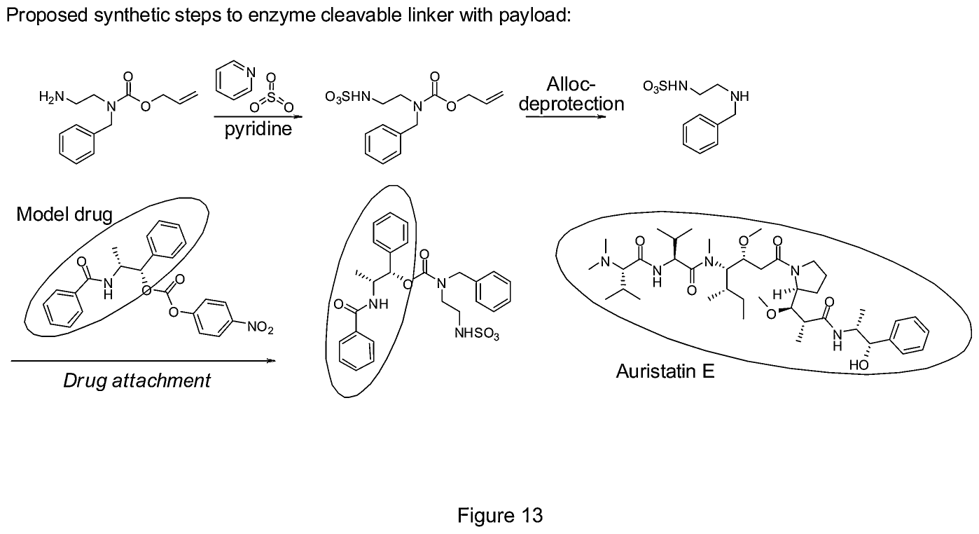

FIG. 12 and FIG. 13 show a synthetic pathway for an enzyme cleavable linker of formula (II) comprising the synthetic procedures outlined below.

### Neopentyl chlorosulfate

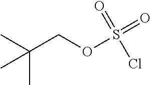

Sulfuryl chloride (4.3 mL, 53.0 mmol, 1 eq.) in dry DCM (20 mL) was cooled to −78 degrees C. in a dry ice/acetone bath in N₂-atmosphere. A solution of neopentyl alcohol (6.68 g, 75.8 mmol, 1.4 eq.) and freshly distilled pyridine (4.3 mL, 53.0 mmol, 1 eq.) in dry DCM (20 mL) was added through a separation funnel dropwise (<1 drop/sec for the first ˜5 mL and then ˜2 drops/sec for the remaining solution). The reaction mixture stirred for 5 hours at RT and was filtered with suction three times and concentrated in vacuo. The crude product was carefully distilled at reduced pressure to give the desired product in yield 69%.

¹H-NMR (400 MHz, CDCl₃): δ=4.23 (s, 2H), 1.10 (s, 9H). ¹³C-NMR (101 MHz, CDCl³-d): δ=85.50, 31.94, 25.90. UPLC-MS (ESI) m/z found 168.8 [M−H₂O]⁻, required 168.0 [C₅H₉ClO₂S]⁻.

### 4-(((Tert-butyldimethylsilyl)oxy)methyl)phenol

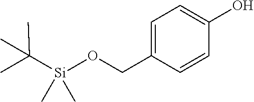 [SMILES: CC(C)(C)[Si](C)(C)OCc1ccc(O)cc1]

To a dry flask, 4-hydroxymethylphenol (5 g, 40.28 mmol, 1 eq.) was dissolved in dry THF (42 mL) and cooled in an ice-bath. Imidazole (5.48 g, 80.56 mmol, 2 eq.) was added and the reaction mixture was left stirring for 10 minutes. Then tert-butylchlorodimethylsilane (7.28 g, 48.33 mmol, 1.2 eq) was added. The reaction stirred at room temperature for 2 hours until LCMS showed full consumption of the starting material. The mixture was quenched with ammonium chloride (15 mL) and extracted with ethyl acetate (3×60 mL). The combined organic layer was dried with anhydrous magnesium sulfate, filtered and concentrated in vacuo. The product was purified by column chromatography on silica gel using 4:1 heptane/EtOAc as eluent to give the desired product 1 in 70% yield.

¹H-NMR (400 MHz, CDCl₃): δ=7.21 (dd, J=21.4, 8.0 Hz, 2H), 6.80 (d, 1H), 4.69 (s, 2H), 0.96 (s, 9H), 0.12 (s, 6H). 13C-NMR (101 MHz, CDCl₃): δ=154.62, 133.50, 127.87, 115.11, 64.83, 26.00, 18.46, 5.15. UPLC-MS (ESI): m/z found 237.0 [M−H]⁻, required 237.1 [C₁₃H₂₁O₂Si]⁻.

### 4-(((Tert-butyldimethylsilyl)oxy)methyl)phenyl neopentyl sulfate

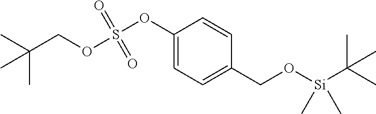 [SMILES: CC(C)(C)COS(=O)(=O)Oc1ccc(CO[Si](C)(C)C(C)(C)C)cc1]

A solution of 4-(((tert-butyldimethylsilyl)oxy)methyl)phenol (2.29 g, 9.62 mmol, 1 eq.) in DMPU (9.6 mL) and THF (28 mL) and was cooled in a dry ice and acetone bath. NaHMDS (10.6 mL, 1 M in THF, 10.56 mmol, 1.1 eq) was added over 4 minutes and the solution was left stirring for 15 minutes before neopentyl chlorosulfate (1.7 mL, 10.56 mmol, 1.1 eq.) was added and the reaction stirred for further 15 minutes in ice bath and 20 minutes at RT. TLC indicated remaining 4-(((tert-butyldimethyl silyl)oxy)-methyl)phenol, so more neopentyl chlorosulfate (0.85 mL, 5.28 mmol, 0.55 eq.) was added and the reaction mixture was left stirring for 10 minutes and further neopentyl chlorosulfate (0.85 mL) was added on the basis of another TLC test. After 10 minutes, the reaction was quenched with NaHCO3(75 mL). The reaction was extracted with EtOAc (4×50 mL) and H2O (2×40 mL) and 50 mL brine. The combined organic layer was dried with anhydrous Na₂SO₄, filtered and concentrated in vacuo. The product was purified by column chromatography on silica gel by gradually eluating with pure heptane to 17:3 heptane/EtOAc. This resulted in isolation of the product 4-(((tert-butyldimethylsilyl)oxy)methyl)phenyl neopentyl sulfate in 65% yield.

¹H-NMR (400 MHz, CDCl₃): δ=7.39 (d, J=8.6 Hz, 2H), 7.29 (d, 2H), 4.76 (s, 2H), 4.10 (s, 2H), 1.02 (s, 9H), 0.97 (s, 9H), 0.13 (s, 6H). ¹³C-NMR (101 MHz, CDCl₃): δ=149.07, 140.73, 127.31, 120.80, 83.37, 64.14, 31.94, 18.39, 5.29. UPLC-MS (ESI): m/z found 387.0 [M−H]⁻, required 387.2 [C₁₈H₃₁O₅SSi]⁻.

### 4-(Hydroxymethyl)phenyl neopentyl sulfate

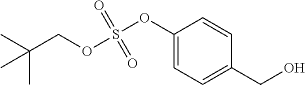 [SMILES: CC(C)(C)COS(=O)(=O)Oc1ccc(CO)cc1]

TBAF (7 mL, 1 M in THF, 6.94 mmol, 1.5 eq.) was added to a solution of 4-(((tert-butyldimethylsilyl)oxy)methyl)phenyl neopentyl sulfate (1.8 g, 4.63 mmol, 1 eq.) in dry THF (10 mL) and left with stirring overnight under N₂ atmosphere. As LCMS indicated consumption of starting material, the reaction was quenched with PBS buffer (35 mL) and diluted with EtOAc (70 mL) the aqueous phase was extracted with EtOAc (3×50 mL) and the combined organic layer washed with H₂O (3×75 mL) and brine (75 mL). The organic layer was dried with anhydrous MgSO₄, filtered and concentrated in vacuo to give 4-(Hydroxymethyl)phenyl neopentyl sulfate in excellent yield of 95%.

¹H-NMR (400 MHz, CDCl₃): δ=7.43 (d, J=8.5 Hz, 2H), 7.34-7.29 (m, 2H), 4.72 (s, 2H), 4.11 (s, 2H), 1.03 (s, 9H). ¹³C-NMR (101 MHz, CDCl₃): δ=149.50, 140.09, 128.31, 121.14, 83.48, 64.36, 31.95, 25.96. UPLC-MS (ESI): m/z found 237.0 [M−H]⁻, required 237.1 [C₁₂H₁₇O₅S]⁻.

### Neopentyl (4-((((4-nitrophenoxy)carbonyl)oxy)methyl)phenyl) sulfate

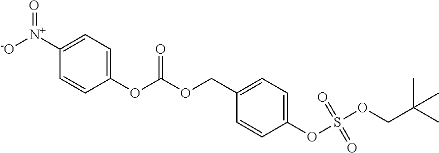

Pyridine dried over KOH (0.35 mL, 4.22 mmol, 1 eq.) and 4-nitrophenyl chloroformate (0.85 g, 4.22 mmol, 1 eq.) was added to a solution of 4-(hydroxymethyl)phenyl neopentyl sulfate (1.16 g, 4.22 mmol, 1 eq.) in dry DCM (12 mL) under a N2 atmosphere and left stirring for 4 hours. The reaction was diluted with DCM (75 mL) and washed with 1 M HCl(aq.) (50 mL), saturated NaHCO₃(aq.) (75 mL), H₂O (5×75 mL) and brine (75 mL). The organic layer was dried with anhydrous MgSO₄, filtered and concentrated in vacuo. The crude product was purified by silica gel chromatography using 7:1 heptane/EtOAc as eluent to obtain neopentyl (4-((((4-nitrophenoxy)carbonyl)oxy)methyl)phenyl) sulfate in yield of 22%.

¹H-NMR (400 MHz, CDCl₃): δ=8.34-8.26 (m, 2H), 7.57-7.49 (m, 2H), 7.43-7.39 (m, 2H), 7.39-7.35 (m, 2H), 5.32 (s, 2H), 4.13 (s, 2H), 1.03 (s, 9H). ¹³C-NMR (101 MHz, CDCl₃-d): δ=171.16, 155.39, 152.36, 150.56, 145.48, 133.44, 130.32, 125.34, 121.76, 121.47, 83.67, 69.79, 60.40, 31.96, 25.96, 21.06, 14.20. UPLC-MS (ESI): m/z found 456.6 [M+H₂O]+, required 457.1 [C₁₉H₂₃NO₁₀S]⁺.

### tert-Butyl (2-(benzylamino)ethyl)carbamate

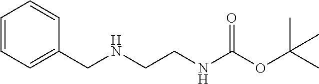 [SMILES: CC(C)(C)OC(=O)NCCNCc1ccccc1]

A solution of N-boc-ethylenediamine (2.0 mL, 12.49 mmol, 1 eq.) and methanol (23 mL) was added anhydrous MgSO₄ (3 g, 24.98 mmol, 2 eq.) and benzaldehyde (1.3 mL, 12.49 mmol, 1 eq.) and left stirring at RT overnight. The solution 4-aminobenzylalcohol (1.0 g, 8.12 mmol, 1.0 eq.) was dissolved in 8.1 mL 6 N HCl and cooled in an ice bath to 0° C. A solution of NaNO₂ was cooled to 0 degrees C. and NaBH4 (0.47 g, 12.49 mmol, 1 eq.) was added portionwise and left stirring for 5 hours before it was quenched with H2O (20 mL). The reaction mixture was extracted with EtOAc (3×50 mL) and the combined organic layer was dried with anhydrous MgSO₄, filtered and concentrated in vacuo. The product was used without further purification.

UPLC-MS (ESI): m/z found 251.2 [M+H]⁺, required 251.2 [C₁₄H₂₃N₂O₂]⁺.

### Allyl benzyl(2-((tert-butoxycarbonyl)amino)ethyl)carbamate

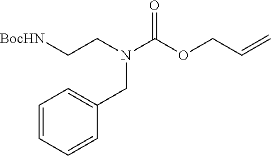 [SMILES: C=CCOC(=O)N(CCNC(=O)OC(C)(C)C)Cc1ccccc1]

tert-Butyl (2-(benzylamino)ethyl)carbamate (3.12 g, 12.48 mmol, 1 eq.) was dissolved in 1:1 dioxane/H2O (50 mL) and cooled to 0 degrees C. NaHCO₃(1.15 g, 13.74 mmol, 1.1 eq.) and allyl chloroformate (3.1 mL, 13.74 mmol, 1.1 eq.) were added. The reaction mixture was left stirring on ice bath for 40 minutes until it was left at room temperature for further 3 hours. Reaction mixture was extracted with EtOAc (3×50 mL) and the combined organic layer was dried with MgSO4, filtered and concentrated in vacuo. Silica filtration using ratio 4:1 heptane/EtOAc to give the desired product in quantitative yield.

¹H-NMR (400 MHz, CDCl₃): δ=7.47-7.15 (m, 6H), 6.10-5.85 (m, 1H), 5.44-5.14 (m, 2H), 4.79-4.58 (m, 3H), 4.55 (s, 2H), 3.47-3.34 (m, 2H), 3.29 (s, 3H), 1.46 (s, 9H). ¹³C-NMR (101 MHz, CDCl₃): δ=156.78, 137.53, 132.79, 128.66, 128.20, 127.92, 127.49, 127.31, 126.98, 117.82, 117.58. UPLC-MS (ESI): m/z found 357.0 [M+Na]⁺, required 357.2 [C₁₈H₂₆N₂NaO_{4]}⁺.

### Allyl (2-aminoethyl)(benzyl)carbamate

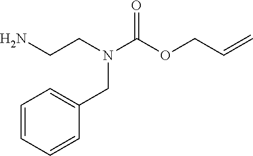 [SMILES: C=CCOC(=O)N(CCN)Cc1ccccc1]

20% TFA in DCM (20 mL) was added to allyl benzyl(2-((tert butoxycarbonyl)amino)ethyl)carbamate (0.15 g, 0.455 mmol, 1 eq.) and was left stirring for 1.5 hours. The reaction mixture was den coevaporated 9 times with THF. The product was used without purification in the next synthesis step.

UPLC-MS (ESI): m/z found 235.3 [M+H]⁺, required 235.1 [C₁₃H₁₉N₂O₂]⁺.

### 4-(7-Benzyl-3,8-dioxo-2,9-dioxa-4,7-diazadodec-11-en-1-yl)phenyl neopentyl sulfate

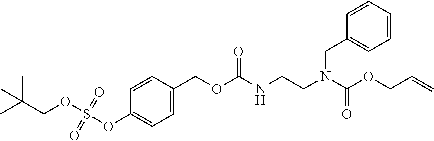 [SMILES: C=CCOC(=O)N(CCNC(=O)OCc1ccc(OS(=O)(=O)OCC(C)(C)C)cc1)Cc1ccccc1]

Allyl (2-aminoethyl)(benzyl)carbamate (0.2 g, 0.455 mmol, 1 eq.) and neopentyl (4-((((4-nitrophenoxy)carbonyl)oxy)methyl)phenyl) sulfate (0.107 g, 0.455 mmol, 1 eq.) were combined in dry THF (4 mL). The solution was added DIPEA until pH was basic (in total 2.2 mL). The reaction was left stirring in N2 atmosphere overnight at RT. The reaction was diluted with 20 mL of EtOAc and washed with H2O (10×20 mL) and brine (2×20 mL) to remove 4-nitrophenol. The organic layer was dried with anhydrous MgSO4, filtered and concentrated in vacuo. The crude product was purified with column chromatography using 8:1 heptane/ethyl acetate as eluent to obtain the title compound in 67% yield.

¹H-NMR (400 MHz, DMSO-d₆): δ=7.49-7.44 (m, 2H), 7.43-7.38 (m, 3H), 7.36-7.17 (m, 6H), 5.35-5.24 (m, 1H), 5.24-5.09 (m, 2H), 5.04 (s, 2H), 4.60-4.51 (m, 2H), 4.51-4.37 (m, 2H), 4.22 (s, 2H), 3.23 (t, J=6.5 Hz, 3H), 3.20-3.07 (m, 2H), 2.50 (p, J=1.8 Hz, 4H), 0.94 (s, 9H). ¹³C-NMR (101 MHz, DMSO-d⁶): δ=156.49, 149.51, 138.37, 137.41, 133.82, 129.87, 128.97, 127.80, 127.62, 127.43, 121.73, 117.20, 83.84, 65.80, 64.79, 50.53, 32.08, 25.91. UPLC-MS (ESI): m/z found 535.6 [M+H]⁺, required 535.2 [C₂₆H₃₅N₂O₈S]⁺.

### Example 11: Stability Tests

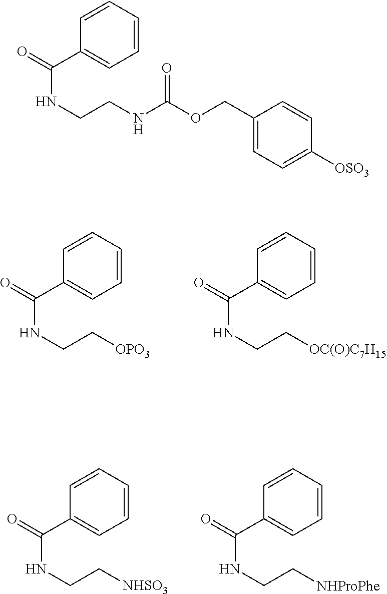

The above compounds were dissolved in a minimum of DMSO and added to a plasma sample, PBS, or MilliQ water and stored at room temperature. Aliquots were taken out at different time points and LC-MS and HPLC used for analysis. LCMS analysis required a small organic extraction of each aliquot in order to remove the protein prior to analysis. Analytical HPLC allowed direct injection and offers high sensitivity and reliability. Caffeine was included as an internal standard. No hydrolysis was observed for any of the compounds after 4 days of incubation.

### Example 12: Enzyme Tests

### Cleavage by Helix pomatia Type H-1

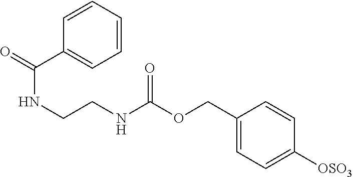

The above compound was dissolved in a minimum of DMSO and added to Helix pomatia Type H-1 in phosphate buffer solutions at pH 5, 6 and 7.4, respectively at room temperature. Aliquots were taken out at different time points and LC-MS and HPLC used for analysis. LCMS analysis required a small organic extraction of each aliquot in order to remove the protein prior to analysis. Analytical HPLC allowed direct injection and offers high sensitivity and reliability. Caffeine was included as an internal standard. The compound was almost fully hydrolysed at 5 min.

### Cleavage by Acid Phosphatase

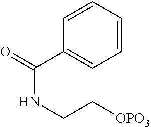

The above compound was dissolved in a minimum of DMSO and added to acid phosphatase in phosphate buffer solutions at pH 5, 6 and 7.4, respectively at room temperature. Aliquots were taken out at different time points and LC-MS and HPLC used for analysis. LCMS analysis required a small organic extraction of each aliquot in order to remove the protein prior to analysis. Analytical HPLC allowed direct injection and offers high sensitivity and reliability. Caffeine was included as an internal standard. The compound was almost fully hydrolysed at 1 hour.

### Cleavage by Human Sulfamidase/SGSH Protein

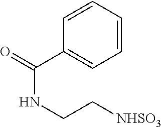

The above compound is dissolved in a minimum of DMSO and added to Human Sulfamidase/SGSH Protein in phosphate buffer solutions at pH 5, 6 and 7.4, respectively at room temperature. Aliquots are taken out at different time points and LC-MS and HPLC used for analysis. LCMS analysis requires a small organic extraction of each aliquot in order to remove the protein prior to analysis. Analytical HPLC allows for direct injection and offers high sensitivity and reliability. Caffeine is included as an internal standard. The compound is almost fully hydrolysed at in 5 min to 1 hour.

### Cleavage by Amano Lipase

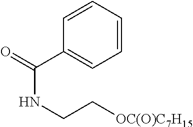

The above compound was dissolved in a minimum of DMSO and added to amano lipase in phosphate buffer solutions at pH 5, 6 and 7.4, respectively at room temperature. Aliquots were taken out at different time points and LC-MS and HPLC used for analysis. LCMS analysis required a small organic extraction of each aliquot in order to remove the protein prior to analysis. Analytical HPLC allowed direct injection and offers high sensitivity and reliability. Caffeine was included as an internal standard. The compound was almost fully hydrolysed at 5 min.

### Cleavage by Dipetidyl Aminopeptidase

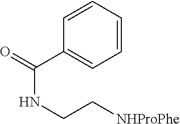

The above compound was dissolved in a minimum of DMSO and added to acid phosphatase in phosphate buffer solutions at pH 5, 6 and 7.4, respectively at room temperature. Aliquots were taken out at different time points and LC-MS and HPLC used for analysis. LCMS analysis required a small organic extraction of each aliquot in order to remove the protein prior to analysis. Analytical HPLC allowed direct injection and offers high sensitivity and reliability. Caffeine was included as an internal standard. The compound was almost fully hydrolysed at 1 hour.

### Example 13: Lipase Linker-Drug Conjugate

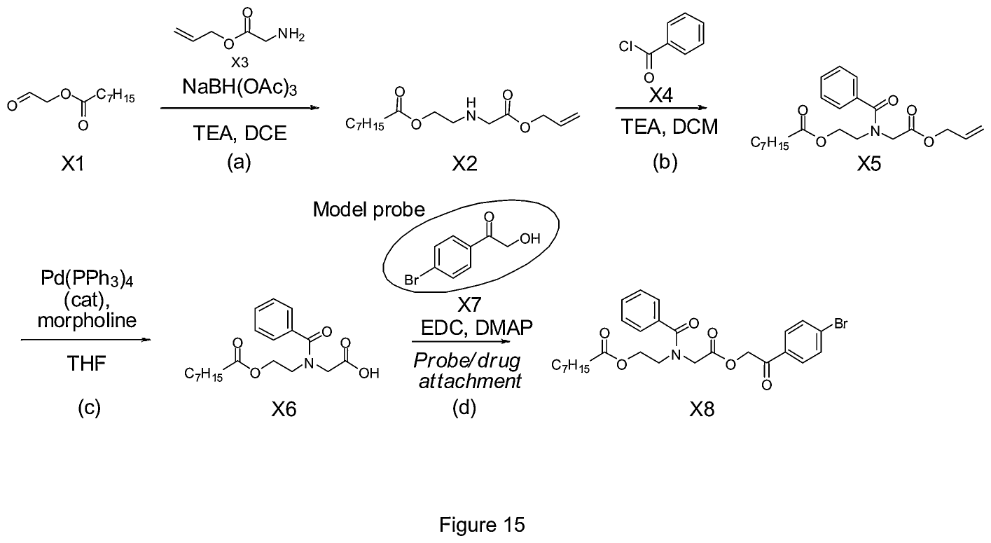

In order to generate the linker X8 (see FIG. 15), the synthetic route of FIG. 15 was undertaken. Late-stage attachment of the model drug (step d) would allow a wide variety of probes to be easily investigated. The bromohydroxyketone X7 was chosen as the model probe to attach to the linker. The presence of the aromatic group would allow the release reaction to be monitored by LCMS. In addition, the bromo-substitution increases the molecular weight, aiding ionisation with a characteristic isotope pattern. A benzoyl group would mimic the biomolecule-attachment point.

### Synthesis

Compound X2 (FIG. 15) was assessed from a reductive amination (as described in synthesis of 2-((2-(allyloxy)-2-oxoethyl)amino)ethyl octanoate). With the secondary amine X2 in hand, amide coupling with benzoyl chloride was attempted using benzoyl chloride and triethylamine, step b (as described in synthesis 2-(N-(2-(allyloxy)-2-oxoethyl)benzamido)ethyl octanoate). The Pd(0)-mediated allyl deprotection (step c) proceeded smoothly to furnish X5 (as described in synthesis of N-benzoyl-N-(2-(octanoyloxy)ethyl)glycine). In order to attach the model probe compound (X7), an EDC/DMAP coupling reaction was employed (as described in synthesis 2-(N-(2-(2-(4-bromophenyl)-2-oxoethoxy)-2-oxoethyl)benzamido)ethyl octanoate).

### Lipase and Stability Tests

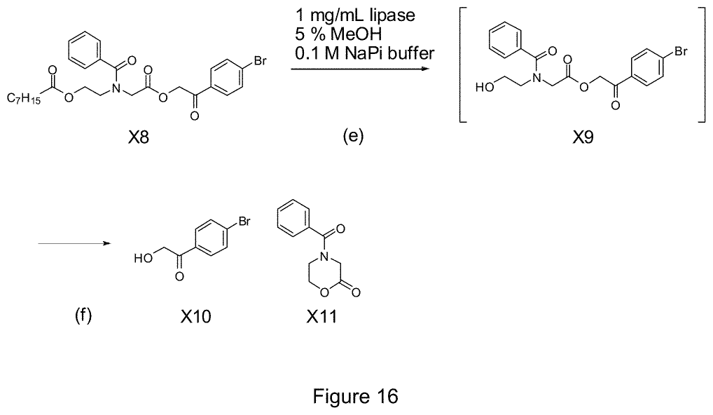

Compound X8 was exposed to lipase in phosphate buffered solutions at pH 6 and 7.4. The chosen lipase was Amano Lipase from Pseudomonas fluorescens, as a useful indicator as to demonstrate the enzyme cleavage and probe release. The pH 6 and 7.4 tests were conducted to determine if any difference in the rate of intramolecular lactonisation would be observed. Initial tests involved incubation of the linker (1 mM in 2% DMSO/buffer) with 1 mg/mL lipase (>20 U/mL) at room temperature (FIG. 16). Caffeine was included as an internal standard. Aliquots were taken out at different time points and LC-MS and HPLC used for analysis.

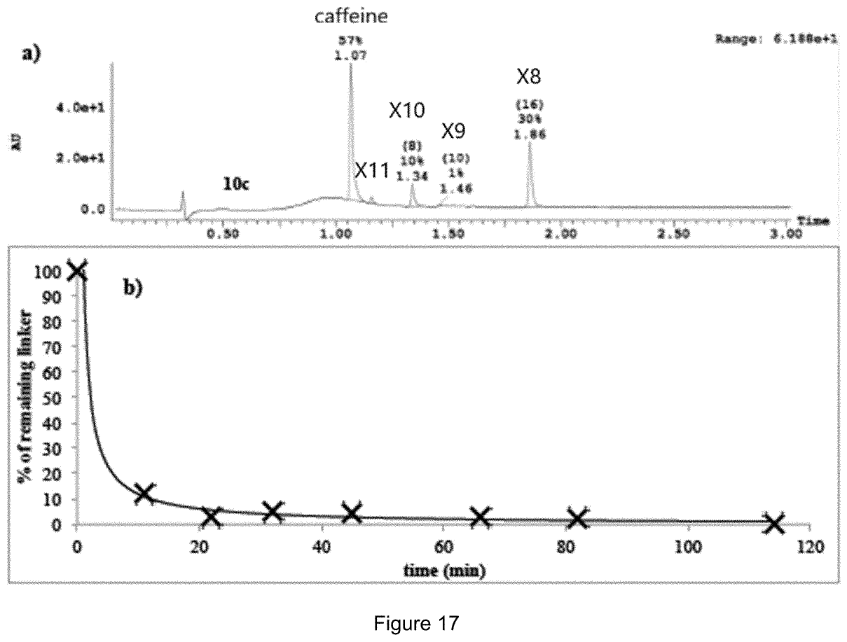

For linker X8 at pH 7.4, full conversion to X10 and X11 was observed in <15 min with no significant accumulation of the intermediate X9. Cleavage of linker X8 showed a non-linear conversion to products over 114 min with negligible accumulation of intermediate X9 (FIG. 17).

Contrastingly, at pH 6, major accumulation of X9 was observed, with full conversion to X10 and X11 only occurring after 60 min, indicating that the rate of cyclisation was clearly lower at the more acidic pH.

### Example 14: Kinetics of Model Prodrug Release Step

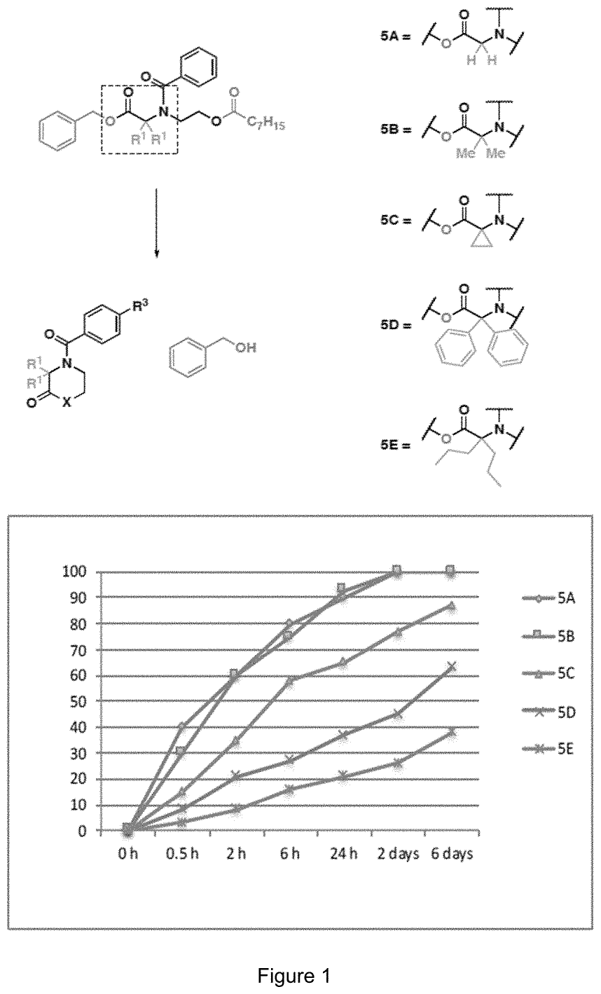

The self-immolative release introduces a second step with its own kinetic parameters. For many applications, the self-immolative step needs to be fast enough in order to ensure a fast release at the activation site. In other applications, a steady and slow release is desired. We have obtained comparative data on the kinetics of the self-immolative step so that anyone can choose a linker suited to their applications (FIG. 1).

Linker compounds were dissolved in a minimum of MeCN and added to lipase in phosphate buffer solutions at pH 7.4 at room temperature. The chosen lipase was the commercially available Amano lipase from Pseudomonas fluorescence. Aliquots were taken out at different time points and LC-MS and HPLC used for analysis. LCMS analysis required a small organic extraction of each aliquot in order to remove the protein prior to analysis. Analytical HPLC allowed direct injection and offers high sensitivity and reliability. Caffeine was included as an internal standard. Accumulation of the intermediate was observed in several linker analogous. The % release of benzyl alcohol was taken as a measure of extend of ring closure (FIG. 1).

### Example 15: Specific Constructs

Trastuzumab—alkyl sulfate—Auristatin E

In an example, biomolecule T is the antibody trastuzumab, the enzyme cleavable moiety is an alkyl sulfate (Y is oxygen, R⁴ is —S(O)₂—OR⁷), the rate determining groups R¹ and R⁶ are hydrogen. The construct binds to HER2, to induce internalization into the target cell. Lysosomal enzymatic cleavage by upregulated steroid sulfatase and subsequent kinetically controlled ring closure would result in the release of R³X, in this specific embodiment the highly cytotoxic Auristatin E. Accordingly the goal of killing breast cancer cells could be achieved swiftly and with high selectivity.

Nanobody-Phosphatase Linker-Drug Conjugate

Due to their inherently simplified structure when compared to full-sized antibodies, nanobodies, such as HER2 specific 2Rs15d, can be readily expressed in E. coli-a host system that enables facile production of nanobodies in large amounts and at a low cost.

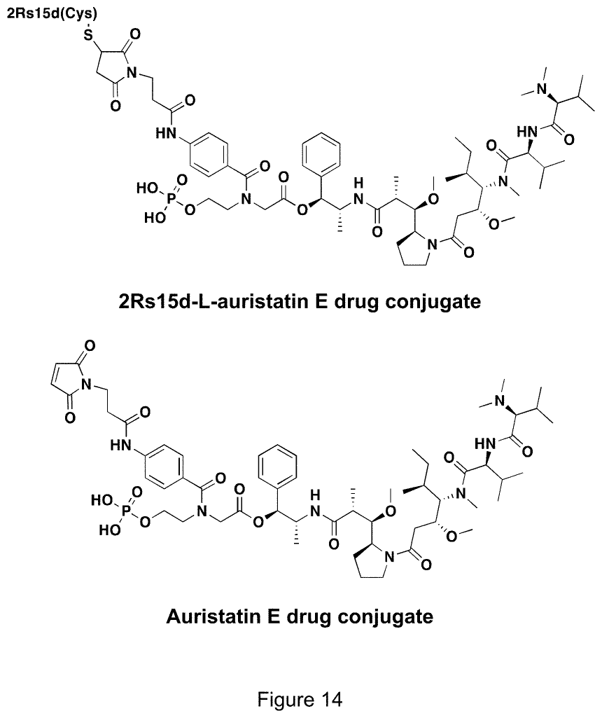

An exemplary structure is 2Rs15d-L-auristatin E drug conjugate (FIG. 14) targeted for breast cancer. Auristatin E works by acting as a tubulin inhibitor intracellularly to arrest cell division. The nanobody 2Rs15d will ensure receptor mediated endocytosis once the drug conjugate binds to HER2 receptor, overexpressed in 20-30% female breast cancer. Once taken up inside the lysosome of a breast cancer cell, a lysosomal phosphatase will cleave the phosphate ester, thereby releasing a free alcohol, which cyclizes and releases the auristatin E.

Production and testing of nanobody drug conjugate—overview. (1) expression of 2Rs15d in E. coli, (2) purification of 2Rs15d, (3) synthesis of a phosphatase cleavable linker (L) covalently attached to auristatin E, (4) evaluation of L-auristatin E (Q) stability in buffer, plasma, and buffer containing phosphatase, the latter serving as a model system for lysosomal phosphatase, (5) bioconjugation of L-auristatin E constructs to 2Rs15d and purification of the final drug conjugate, and (7) in vitro testing of the drug conjugate.

Expression. Biosynthesis of 2Rs15d is performed in E. coli SHuffle® T7 Competent cells able to manufacture the two disulfide bonds present in the 2Rs15d peptide core. The sequence coding for 2Rs15d is cloned into pET-22b(+) vector, which is transfected into E. coli SHuffle® T7 Competent cells. The cells are grown in LB medium with 0.1% glucose and 1 mM MgCl₂ at 37° C. (+ampicilin). After OD₆₀₀ₙₘ reaches 0.7, induction of expression is realized by adding 250 μL IPTG stock (1 M) (for 0.5 L culture). Growth and expression are continued for 4 hours at 37° C. with shaking (180-250 rpm). After expression, cells are harvested by centrifugation for 15 min at 9,000 xg, supernatant discarded, and pellet stored at −20° C.

Work up and purification. Cell pellet is thawed for 15 min on ice and resuspended in 10 mL lysis buffer, after which lysozyme (final concentration of 1 mg/mL) and Benzonase® Nuclease solution (final concentration: 25 U/mL) are added. The mixture is incubated on ice for 30 min to achieve complete cell lysis. Lysate is centrifuged at 14,000×g for 30 min at 4° C. to pellet cellular debris. Supernatant containing desired soluble proteins is directly loaded onto Fast Start Columns for purification. After wash, bound His-tagged 2Rs15d is eluted 2 times with elution buffer. The desired fractions containing crude 2Rs15d are subjected to size exclusion chromatography. Purity of purified 2Rs15d is confirmed by SDS page (4-20% Mini-PROTEAN TGX Precast Protein Gels) using appropriate ladder (Precision Plus Protein™ Dual Xtra Prestained Protein Standards). The molecular weight of 2Rs15d is further confirmed by ESI-HRMS.

Synthesis. Synthetic steps towards a a phosphatase cleavable linker is described in detail in Example 9.

Stability and release of auristatin E from auristatin E drug conjugate. (a) 0.1 mg auristatin E drug conjugate (FIG. 14) is dissolved in 0.5 mL 1x PBS (137 mM NaCl, 10 mM phosphate, 2.7 mM KCl, and a pH of 7.4) in a 1.5 mL Eppendorf tube fitted with a magnet. The solution is stirred at 37° C., and samples are withdrawn (50 μL) at various time intervals and quenched in liquid nitrogen. Stability is assessed by LCMS, readout is area under the curve (AUC). (b) 0.1 mg auristatin E drug conjugate is dissolved in 0.5 mL human plasma (Sigma Aldrich, P9523) in a 1.5 mL Eppendorf tube fitted with a magnet. The solution is stirred at 37° C., and samples are withdrawn (50 μL) at various time intervals and quenched in liquid nitrogen. Stability is assessed by LCMS, readout is area under the curve (AUC). (c) 0.1 mg auristatin E drug conjugate is dissolved in 0.5 mL 1x PBS containing in a 1.5 mL Eppendorf tube fitted with a magnet, and this mixture is charged with purified recombinant human lysosomal acid phosphatase (ACP2) (MyBioSource, MBS7111128). The solution is stirred at 37° C., and samples are withdrawn (50 μL) at various time intervals and quenched in liquid nitrogen. Stability is assessed by LCMS, readout is area under the curve (AUC).

Bioconjugation of auristatin E drug conjugate to 2Rs15d to give 2Rs15d-L-auristatin drug conjugate and purification. This procedure is in accordance with literature (Bioconjugate Chem. 2014, 25, 979-988). The maleimide functional group is reacted with the terminal cysteine on 2Rs15d. Prior to bioconjugation and to liberate free cysteines from potential dimer 2Rs15d, 2Rs15d is subjected to mild reducing conditions by subjecting it to 2-mercaptoethylamine (2-MEA, 180 equivalents) at a concentration of 1 mg/mL 2Rs15d, pH 7.4 for 90 min at 37° C. Linker construct auristatin E drug conjugate (10 equivalents) dissolved in 0.2 M NH₄₀Ac (pH 6.0) is added to freshly reduced 2Rs15d to obtain the desired nanobody-drug conjugate. Finally, the nanobody-drug conjugate is purified using SEC.

In vitro testing of 2Rs15d-L-auristatin E drug conjugate. The internalization properties of 2Rs15d-L-auristatin E drug conjugate are analyzed by fluorescent marking (Alexa Fluor 488, 532, or 647) of the 2Rs15d-L-auristatin E drug conjugate. Using fluorescence confocal microscopy, receptor-specific internalization the 2Rs15d-L-auristatin E drug conjugate in HER2-positive SK-BR-3 and BT-474 human BC cell lines (HER-negative cells are used as control) is evaluated. Prior to adding the 2Rs15d-L-auristatin E drug conjugate, cells are treated with proteases to strip membrane proteins from the cell surface. Fluorescently marked constructs will also be subjected to flow cytometry to measure cell surface binding to HER2. Next, the cytotoxic effects of the 2Rs15d-L-auristatin E drug conjugate is evaluated; cells are subjected to a dilution series of the 2Rs15d-L-auristatin E drug conjugate. Efficacy of the 2Rs15d-L-auristatin E drug conjugate is estimated based on MTS cytotoxic assay (or other appropriate viability assays) using cell count as readout.

### Example 16: Synthesis of a Phosphatase Cleavable Linker of Formula (I)

The procedures below outline the synthesis of a phosphatase cleavable linker.

### tert-Butyl (2-((bis(benzyloxy)phosphoryl)oxy)ethyl)glycinate

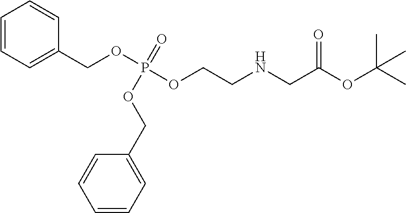 [SMILES: CC(C)(C)OC(=O)CNCCOP(=O)(OCc1ccccc1)OCc1ccccc1]

In a pre-dried round-bottom flask, tert-butyl (2-hydroxyethyl)glycinate (0.40 g, 2.26 mmol) was dissolved in anhydrous DCM (60 mL), added dibenzyl N,N-diisopropylphosphoramidite (0.75 mL, 2.26 mmol) and tetrazole (0.45 M in MeCN, 5.07 mL), and the reaction was allowed to stir at rt for 4 h. The reaction mixture was cooled to 0° C., added Luperox® TBH70X (70 wt. % in water, 2.23 mL), then brought to rt and stirred vigorously overnight at rt under nitrogen atmosphere. The reaction was diluted in DCM (50 mL), the organic phase washed with sat. NaHCO₃(2×40 mL), dried over Na₂SO₄, and the title compound was obtained after flash chromatography (EtOAc→EtOAc+5% MeOH) as a clear oil (502 mg, 51%).

R_{f}=0.50 (EtOAc+2% MeOH); ¹H NMR (400 MHz, Chloroform-d) δ 7.37-7.32 (m, 10H), 5.08-5.02 (m, 4H), 4.11 (dt, J=7.5, 5.2 Hz, 2H), 3.31 (s, 2H), 2.86 (t, J=5.2 Hz, 2H), 2.02 (s, 1H), 1.45 (s, 9H); UPLC-MS (ESI) m/z found 436.2 [M+H]⁺, calc. 436.19 [C₂₂H₃₁NO₆P]⁺.

### tert-Butyl N-(4-azidobenzoyl)-N-(2-((bis(benzyloxy)phosphoryl)oxy)ethyl)glycinate

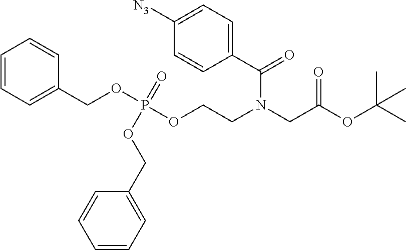 [SMILES: CC(C)(C)OC(=O)CN(CCOP(=O)(OCc1ccccc1)OCc1ccccc1)C(=O)c1ccc(N=[N+]=[N-])cc1]

In a pre-dried round-bottom flask, tert-butyl (2-((bis(benzyloxy)phosphoryl)oxy)ethyl)glycinate (429 mg, 0.98 mmol) was dissolved in anhydrous DCM (6.4 mL), added DIPEA (0.52 mL, 2.95 mmol) and DMAP (12 mg, 0.10 mmol), the reaction mixture was cooled to 0° C. and stirred for 15 min, after which 4-azidobenzoyl chloride (2.2 M in anhydrous DCM, 0.5 mL) was added dropwise. The reaction mixture was allowed to slowly reach rt and stirred overnight under nitrogen atmosphere, then diluted in DCM (40 mL), added sat. NaHCO₃(25 mL), the aqueous phase was extracted with DCM (3×25 mL), the organic phase dried over Na₂SO₄, and the title compound was obtained after flash chromatography (EtOAc/PE, 1:1) as a clear oil (272 mg, 48%).

R_{f}=0.20 (EtOAc/PE, 1:1); ¹H NMR (400 MHz, Chloroform-d) δ 7.33 (app. s, 12H), 6.96 (d, J=8.2 Hz, 2H), 5.13-4.90 (m, 4H), 4.30-3.42 (m, 6H), 1.47* (s, 3H), 1.40 (s, 6H); UPLC-MS (ESI) m/z found 581.6 [M+H]⁺, calc. 581.22 [C₂₉H₃₄N₄O₇P]⁺.

### tert-Butyl N-(4-aminobenzoyl)-N-(2-(phosphonooxy)ethyl)glycinate

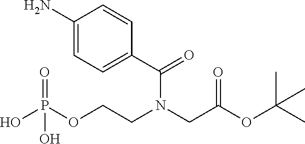 [SMILES: CC(C)(C)OC(=O)CN(CCOP(=O)(O)O)C(=O)c1ccc(N)cc1]

In a pre-dried round-bottom flask, tert-butyl N-(4-azidobenzoyl)-N-(2-((bis(benzyloxy)phosphoryl)oxy)ethyl)glycinate (61 mg, 0.11 mmol) was dissolved in freshly distilled MeOH (1.5 mL), the flask was flushed with nitrogen, added 5% Pd/C (2.5 mg), re-evacuated with hydrogen (×3), and the reaction was stirred under hydrogen atmosphere at rt for 4 h. The reaction mixture was filtered and the solvent removed to give the title compound as a light yellow solid (33 mg, 84%).

¹H NMR (400 MHz, DMSO-d₆) δ 7.08 (app. s, 2H), 6.54 (d, J=8.2 Hz, 2H), 4.03 (s, 2H), 3.96 (q, J=7.4, 6.9 Hz, 2H), 3.55 (t, J=6.0 Hz, 2H), 1.40 (s, 9H); UPLC-MS (ESI) m/z found 372.9 [M]⁻, calc. 373.12 [C₁₅H₂₂N₂O₇P]⁻.

### N-(4-(3-(2,5-dioxo-2,5-dihydro-1H-pyrrol-1-yl)propanamido)benzoyl)-N-(2-(phosphonooxy)ethyl)glycine

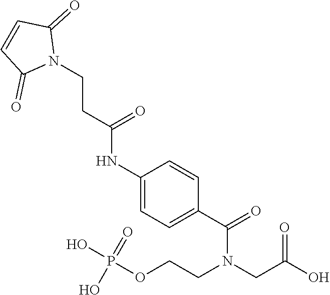 [SMILES: O=C(O)CN(CCOP(=O)(O)O)C(=O)c1ccc(NC(=O)CCN2C(=O)C=CC2=O)cc1]

In a pre-dried microwave vial, a solution of tert-butyl N-(4-aminobenzoyl)-N-(2-(phosphonooxy)ethyl)glycinate (24 mg, 65 μmol), 3-maleimidepropionic acid (10 mg, 60 μmol), N,N,N′,N′-tetramethylchloroformamidinium hexafluorophosphate (TCFH) (18 mg, 65 μmol) in anhydrous DMF (0.2 mL) was added N-methylimidazole (10 μL, 0.13 mmol), and the reaction was left to stir at rt for 20 h. The crude reaction mixture was purified directly using preparative HPLC (linear gradient) and lyophilized yielding the desired product (4 mg, 13%) as a white solid.

¹H NMR (400 MHz, DMSO-d₆) δ 7.63-7.52 (m, 2H), 7.40-7.20 (m, 2H), 7.02 (s, 2H), 4.12-3.61 (m, 8H), 2.60 (t, J=7.1 Hz, 2H), 1.43 (s, 6H), 1.35* (s, 3H); UPLC-MS (ESI) m/z found 526.7 [M+H]⁺, calc. 526.16 [C₂₂H₂₉N₃O₁₀P]⁺.

The title compound was synthesized from the corresponding tert-butyl ester. In a pre-dried microwave vial, tert-butyl ester (3 mg, 6 μmol) dissolved in anhydrous DCM (1 mL) was slowly added TFA (0.16 mL), and the reaction was stirred at rt for 1.5 h. The title compound was concentrated on nitrogen flow to give a light brown solid (2.6 mg, 97%).

UPLC-MS (ESI) m/z found 468.5 [M−H]⁻, calc. 468.08 [C₁₈H₁₉N₃O₁₀P]⁺.

### Example 17: Synthesis of an Sulfatase Cleavable Linker of Formula (I)

The procedures below outline the synthesis of a sulfatase cleavable linker.

### tert-Butyl N-(4-(((((9H-fluoren-9-yl)methoxy)carbonyl)amino)methyl)benzoyl)-N-(2-(sulfooxy)ethyl) glycinate

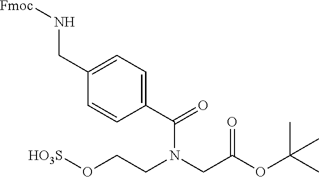

tert-Butyl N-(4-(((((9H-fluoren-9-yl)methoxy)carbonyl)amino)methyl)benzoyl)-N-(2-hydroxyethyl) glycinate (500 mg, 0.94 mmol) and sulfur trioxide pyridine complex (165 mg, 1.04 mmol) was suspended in pyridine (1 mL) and stirred at rt for 1 h. Pyridine was removed by co-evaporation (toluene) and the crude was purified by flash column chromatography (EtOAc→EtOAc+10% MeOH, 10% AcOH) to yield the title compound as a white sticky solid (494 mg, 86%).

¹H NMR (400 MHz, DMSO-d₆) δ 8.91-8.84 (m, 1H), 8.46 (tt, J=7.8, 1.6 Hz, 1H), 8.03-7.93 (m, 2H), 7.89 (d, J=7.6 Hz, 3H), 7.70 (t, J=6.4 Hz, 2H), 7.42 (t, J=7.4 Hz, 2H), 7.37-7.22 (m, 5H), 4.37 (dd, J=6.8, 3.9 Hz, 2H), 4.22 (q, J=7.1, 5.8 Hz, 3H), 4.10 (s, 1H), 3.98 (s, 1H), 3.93 (t, J=5.8 Hz, 1H), 3.80 (t, J=5.9 Hz, 1H), 3.60 (t, J=5.8 Hz, 1H), 3.40 (t, J=5.9 Hz, 1H), 1.45 (s, 5H), 1.34 (s, 3H). UPLC-MS (ESI) m/z found 608.9 [M−H]⁻, calc. 609.7 [C₃₁H³³N₂O₉S]⁻.

### tert-Butyl N-(4-((3-(2,5-dioxo-2,5-dihydro-1H-pyrrol-1-yl) propanamido) methyl) benzoyl)-N-(2-sulfo oxy) ethyl) glycinate

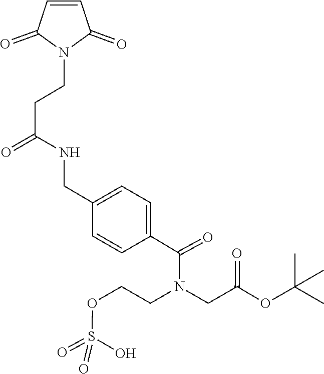 [SMILES: CC(C)(C)OC(=O)CN(CCOS(=O)(=O)O)C(=O)c1ccc(CNC(=O)CCN2C(=O)C=CC2=O)cc1]

tert-Butyl N-(4-(((((9H-fluoren-9-yl)methoxy)carbonyl)amino)methyl)benzoyl)-N-(2-hydroxyethyl) glycinate (273 mg, 0.45 mmol) was dissolved in diethylamine and DCM (1:1, 5 mL) and stirred at rt until complete Fmoc deprotection (25 min). The reaction was added THF and concentrated in vacuo. Crude tert-butyl N-(4-(aminomethyl)benzoyl)-N-(2-(sulfooxy)ethyl)glycinate was dissolved in anhydrous DCM (10 mL) and added 3-(2,5-dioxo-2,5-dihydro-1H-pyrrol-1-yl)propanoic acid (75 mg, 0.45 mmol), HATU (204 mg, 0.54 mmol) and DIPEA (389 μL, 2.24 mmol). The reaction was stirred at rt for 3 h under nitrogen atmosphere. The solvent was removed in vacuo and the crude was purified by flash column chromatography (EtOAc→EtOAc+10% MeOH, 10% AcOH) to yield the title compound as a colorless amorphous solid (93 mg, 39%).

¹H NMR (400 MHz, Chloroform-d) δ 7.41 (d, J=7.7 Hz, 1H), 7.33 (d, J=7.8 Hz, 1H), 7.26-7.20 (m, 1H), 6.69 (d, J=6.5 Hz, 2H), 6.26 (d, J=32.6 Hz, 1H), 4.40 (d, J=5.8 Hz, 2H), 4.33 (t, J=5.2 Hz, 1H), 4.23 (s, 1H), 4.14-4.01 (m, 3H), 3.85 (t, J=7.0 Hz, 3H), 3.59 (t, J=5.8 Hz, 1H), 2.59 (t, J=7.0 Hz, 2H), 1.49 (s, 5H), 1.41 (s, 6H). UPLC-MS (ESI) m/z found 537.8[M−H]⁻, calc. 538.2 [C₂₃H₂₈N₃O₁₀S]⁻.

### Example 18: Conjugation to Cytotoxic Agents Targeting DNA Replication

Conjugation of cytotoxic agents, such as querticin (CytI), PNU-159682 (CytII), camptothecin (CytIII), auristatin E (CytIV), and cryptothycin (CytV), to the enzyme cleavable linkers is carried out as outlined. The cytotoxic agents target DNA replication i.e. by DNA intercalation, DNA topoisomerase I inhibition, or tubulin inhibition.

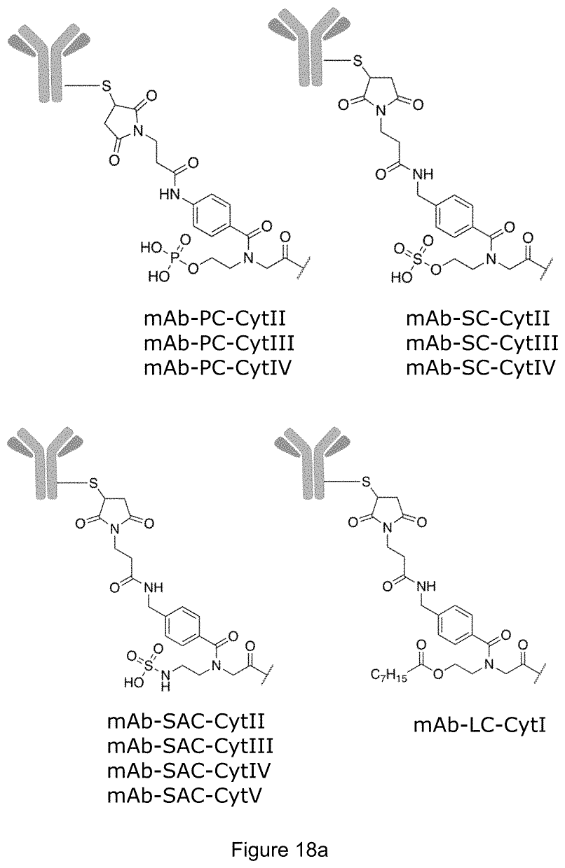

Enzyme cleavable linker, such as a phosphatase cleavable linker (PC), a sulfatase cleavable linker (SC), or a sulfamidase cleavable linker) bearing a free carboxylic acid functionality and a maleimide functionality (PC, SC or SAC) (1 equivalent) is transferred to a pre-dried round bottom flask. Anhydrous DCM is added, and the suspension is cooled by means of an ice bath. To the pre-cooled suspension of enzyme cleavable linker, the cytotoxic agent bearing free hydroxy function (Cyt-II, Cyt-III or Cyt-IV, FIG. 18 (2 equivalents) and DIPEA (4 equivalents) are added, and the reaction mixture is stirred at rt for 5 min. In another pre-dried round bottom flask, BTFFH (fluoro-N,N,N′,N′-bis(tetramethylene) formamidinium hexafluorophosphate) coupling reagent (1.5 equivalent) is dissolved in anhydrous DCM, after which the BTFFH solution is added slowly to the stirring suspension of enzyme cleavable linker and cytotoxic agent. The reaction is left stirring overnight at rt under inert atmosphere, then concentrated on nitrogen flow, purified using preparative HPLC, and lyophilized to give the desired payload such as PC-CytII, PC-CytIII, PC-CytIV, SCCyt-II, SC-CytIII, SC-CytIV, SAC-CytII, SAC-CytIII, or SAC-CytIV). In particular, these conjugates comprise an ester bond between the carboxylic acid moiety of the enzyme cleavable linker and a hydroxy group of the cytotoxic agent.

The same procedure is used to access payloads with amine-containing drugs (e.g. CytV), such as SAC-CytV. In particular, these conjugates comprise an amide bond between the carboxylic acid moiety of the enzyme cleavable linker and an amine group of the cytotoxic agent.

### Example 19: Bioconjugation of Payload to Antibody

The following bioconjugation procedure is used to install one or more linker-drug conjugates (such as PC-CytII, PC-CytIII, PC-CytIV, SC-CytII, SC-CytIII, SC-CytIV, SAC-CytII, SAC-CytIII, SAC-CytIV, or LC-CytI) onto an antibody of choice.

The antibody (1 equivalent) is dissolved in Tris-buffered saline and TCEP (tris(2-carboxyethyl)-phosphine) (50 equivalents) is added. The reaction is incubated at 37° C. for 2 h. A solution of payload (8 equivalent) in DMSO (10% v/v) is added, the reaction is gently shaked for 1 h at rt, and then quenched with N-acetyl cysteine (32 equivalents). Next, the crude antibody-drug-conjugate (ADC) is subjected to spin filtration (40 K MWCO) and then size-exclusion chromatography to yield the isolated antibody (mAb) conjugated to one or more linker-drug conjugates. Examples of ADCs obtainable by this method are: mAb-PC-CytII, mAb-PC-CytIII, mAb-PC-CytIV, mAb-SC-CytII, mAb-SC-CytIII, mAb-SC-CytIV, mAb-SAC-CytII, mAb-SAC-CytIII, mAb-SAC-CytIV, mAb-SAC-CytV, or mAb-LC-CytI (FIG. 18), with varying drug-to-antibody ratios.

A similar procedure is used to access a series of sulfamidase cleavable camptothecin-based payloads such as mAb-SAC-CytIII-a (R═H), mAb-SAC-CytIII-b (R=Me), mAb-SAC-CytIII-c (R=Et), mAb-SAC-CytIII-d (R═Pr), mAb-SAC-CytIII-e (R═Bn), mAb-SAC-CytIII-f (R═Ph), mAb-SAC-CytV-a (R═H), mAb-SAC-CytV-b (R=Me), mAb-SAC-CytV-c (R=Et), mAb-SAC-CytV-d (R═Pr), mAb-SAC-CytV-e (R═Bn), and mAb-SAC-CytV-f (R═Ph), wherein the functional groups R dictate drug release rate (FIGS. 18-20).

### Example 20: Release Rate of Camptothecin

The following protocol can be used to assess the release rate of camptothecin from sulfamidase cleavable ADCs mAb-SAC-CytIII-a (R═H), mAb-SAC-CytIII-b (R=Me), mAb-SAC-CytIII-c (R=Et), mAb-SAC-CytIII-d (R═Pr), mAb-SAC-CytIII-e (R=Bn), and mAb-SAC-CytIII-f (R=Ph).

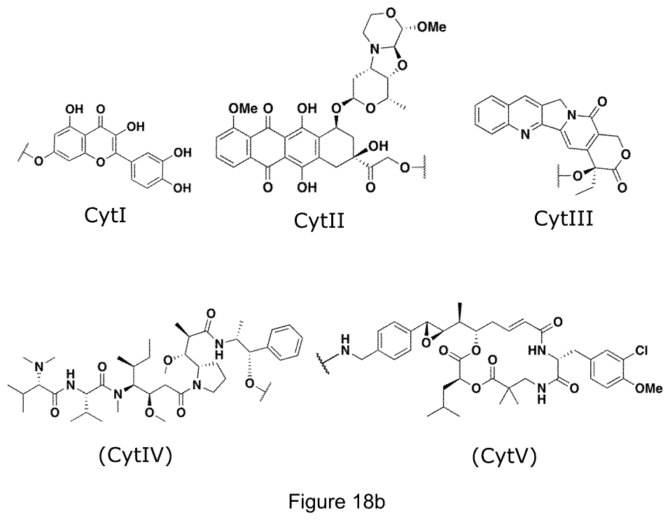

To a 1.5 mL Eppendorf tube containing 150 uL 20 mM ADC in 50 mM IVIES, pH 5.5 assay buffer, 150 uL recombinant human sulfamidase/SGSH (rhSGSH) (Catalog #8380-SU, R&D Systems) (40 μg/mL, in assay buffer) is added and the reaction is incubated at 37° C. with gentle shaking. Samples (20 uL aliqoutes) are withdrawn at different time points (5 min, 30 min, 60 min, 3 h, 6 h, 12 h, 24 h, 48 h, and 72 h), immediately quenched in 2 M NaOH (20 uL), and the extent of reaction (drug release) is analyzed by LCMS (5 uL injections). The moiety in position R affects the rate of release of camptothecin: larger and bulky moieties in the position of R (FIG. 19) such as Ph and Bn provides for a slower release of camptothecin. In comparison, smaller moieties in the position of R provides for a faster release of camptothecin.

### Example 21: Release Rate of Cryptothecin

The following protocol can be used to assess the release rate of cryptothecin from sulfamidase cleavable ADCs mAb-SAC-CytV-a (R═H), mAb-SAC-CytV-b (R=Me), mAb-SAC-CytV-c (R=Et), mAb-SAC-CytV-d (R═Pr), mAb-SAC-CytV-e (R=Bn), and mAb-SAC-CytV-f (R=Ph).

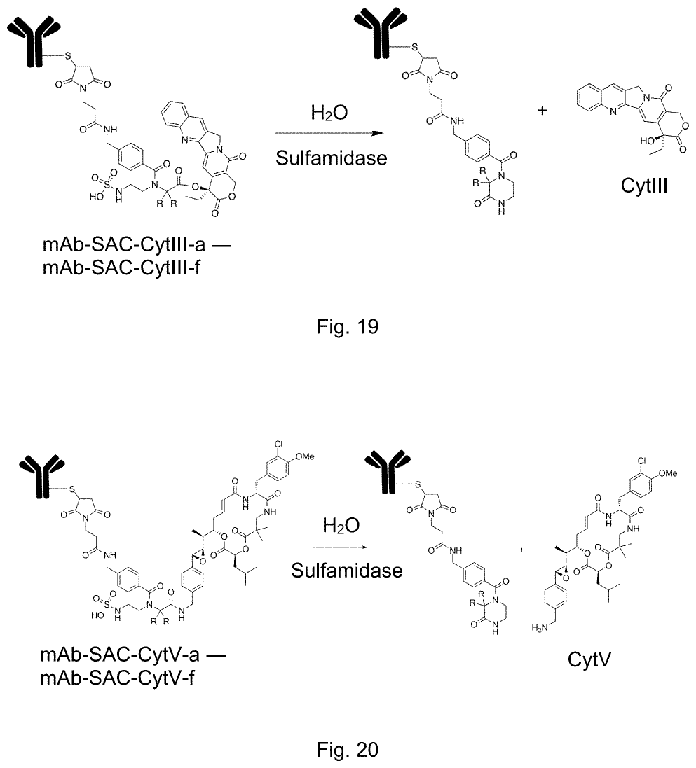

To a 1.5 mL Eppendorf tube containing 150 uL 20 mM ADC in 50 mM IVIES, pH 5.5 assay buffer, 150 uL recombinant human sulfamidase/SGSH (rhSGSH) (Catalog #8380-SU, R&D Systems) (40 μg/mL, in assay buffer) is added and the reaction is incubated at 37° C. with gentle shaking. Samples (20 uL aliqoutes) are withdrawn at different time points (5 min, 30 min, 60 min, 3 h, 6 h, 12 h, 24 h, 48 h, and 72 h), immediately quenched in 2 M NaOH (20 uL), and the extent of reaction (drug release) is analyzed by LCMS (5 uL injections). The moiety in the position R affects the rate of release of cryptothecin: larger and bulky moieties in the position of R (FIG. 20) such as Ph and Bn provides for a slower release of cryptothecin. In comparison, smaller moieties in the position of R provides for a faster release of cryptothecin.

### Example 22: Release Rate of Model Drug

The following protocol can be used to assess the release rate of model drug (benzyl alcohol) from sulfamidase cleavable ADC mAb-SAC2-BnOH.

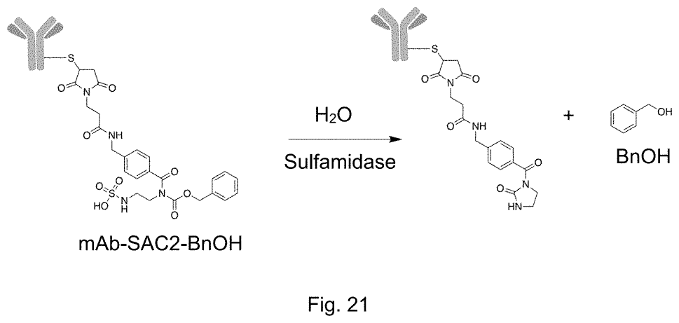

To a 1.5 mL Eppendorf tube containing 150 uL 20 mM ADC in 50 mM IVIES, pH 5.5 assay buffer, 150 uL recombinant human sulfamidase/SGSH (rhSGSH) (Catalog #8380-SU, R&D Systems) (40 μg/mL, in assay buffer) is added and the reaction is incubated at 37° C. with gentle shaking. Samples (20 uL aliqoutes) are withdrawn at different time points (5 min, 30 min, 60 min, 3 h, 6 h, 12 h, 24 h, 48 h, and 72 h), immediately quenched in 2 M NaOH (20 uL), and the extent of reaction (sulfamidase cleavage followed by BnOH release) is analyzed by LCMS (5 uL injections). The rate of cleavage by the sulfamidase to form the free amine is fast, proceeding almost instantly or over a few or several minutes. The subsequent ring closure and release of BnOH is comparatively slower, proceeding over several minutes or several hours (FIG. 21).

What is claimed is:

1. A compound of formula (I), 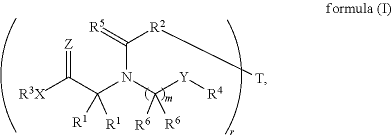
wherein:
each R¹ is independently selected from hydrogen, alkyl, substituted alkyl, aryl, substituted aryl, heteroaryl, or substituted heteroaryl or both R¹ together with the carbon to which they are attached form a cycloalkyl, substituted cycloalkyl, cycloheteroalkyl, or substituted cycloheteroalkyl;
R² is a bond or linking group;
R³ is a moiety comprising at least 19 atoms;
X is >N—, ═N—, —N(H)—, —O—, or —S—;
Y is —O— or —N(R⁸)—;
if Y is —O—, R³X is R³—O—, or R³—S—; whereas
if Y is —N(R⁸)—, R³X is R³>N—, R³═N—, R³—N(H)—, R³—O— or R³—S—;
R⁴ is: 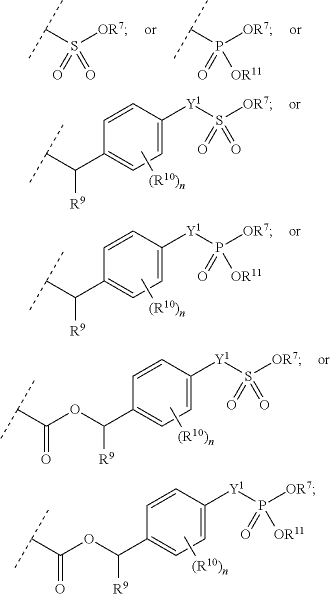
Y¹ is —O— or —N(R⁸)—;
R⁵ is ═O or ═NR¹²;
each R⁶ is independently selected from hydrogen, alkyl, substituted alkyl, aryl, substituted aryl, heteroaryl, or substituted heteroaryl, or any two R⁶ attached to the same carbon form a cycloalkyl, substituted cycloalkyl, cycloheteroalkyl, or substituted cycloheteroalkyl together with the carbon to which they are attached;
R⁷ is a negative charge or hydrogen;
each R⁸ is independently selected from hydrogen, alkyl, substituted alkyl, aryl, or substituted aryl;
R⁹ is selected from hydrogen, alkyl, substituted alkyl, aryl, or substituted aryl;
each R¹⁹ is independently selected from hydrogen, alkyl, substituted alkyl, aryl, or substituted aryl;
R¹¹ is a negative charge, hydrogen, alkyl, substituted alkyl, aryl, or substituted aryl;
R¹² is selected from hydrogen, alkyl, substituted alkyl, aryl, or substituted aryl;
Z is —O— or —S—;
T is a moiety containing at least 19 atoms;
m is 2 to 4;
n is 0 to 4;
r is 1 or more;
any R¹ is optionally together with any R⁶ a bond, an alkylene group, or a heteroalkylene group where such connection results in a cis-configuration of the substituent R³XC(═Z)—and the substituent —(C(R⁶)₂)ₖYR⁴ where k is 0 to m−1;
or a pharmaceutically acceptable salt thereof.

2. The compound according to claim 1 wherein both R¹ are the same and selected from hydrogen, methyl, ethyl, propyl, butyl, pentyl, hexyl, phenyl, or benzyl, or both R¹ taken together is —CH₂CH₂—.

3. The compound according to any one of the preceding claims, wherein both R¹ are hydrogen.

4. The compound according to any one of the preceding claims, wherein both R¹ are methyl.

5. The compound according to any one of the preceding claims, wherein both R¹ are propyl.

6. The compound according to any one of the preceding claims, wherein both R¹ are phenyl.

7. The compound according to any one of the preceding claims, wherein both R¹ taken together is —CH₂CH₂—.

8. The compound according to any of one the preceding claims, wherein R⁶ is independently selected from hydrogen, methyl, ethyl, propyl, butyl, pentyl, hexyl, phenyl, or benzyl, or, two R⁶ attached to the same carbon taken together is —CH₂CH₂—.

9. The compound according to any one of the preceding claims, wherein m is 2.

10. The compound according to any one of the preceding claims, wherein R⁶ is hydrogen.

11. The compound according to any one of the preceding claims, wherein Y is —O— or Y is —N(H)— and R⁴ is

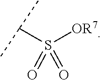

12. The compound according to any one of the preceding claims, wherein Y is —O— or Y is —N(H)— and R⁴ is

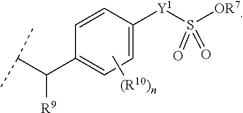

13. The compound according to any one of the preceding claims, wherein Y is —O— or Y is —N(H)— and R⁴ is

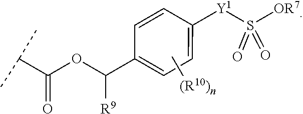

14. The compound according to any one of the preceding claims, wherein Y is —O— or Y is —N(H)— and R⁴ is

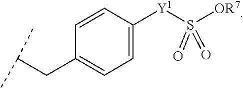

15. The compound according to any one of the preceding claims, wherein Y is —O— or Y is —N(H)— and R⁴ is

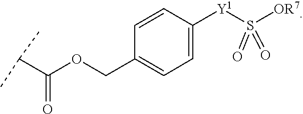

16. The compound according to any one of the preceding claims, wherein YR⁴ taken together is

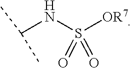

17. The compound according to any one of the preceding claims, wherein YR⁴ taken together is

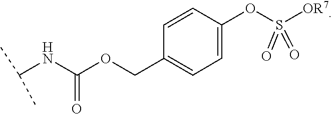

18. The compound according to any one of the preceding claims, wherein Y is —O— or Y is —N(H)— and R⁴ is

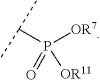

19. The compound according to any one of the preceding claims, wherein Y is —O— or Y is —N(H)— and R⁴ is

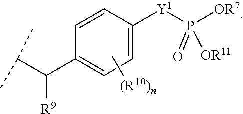

20. The compound according to any one of the preceding claims, wherein Y is —O— or Y is —N(H)— and R⁴ is

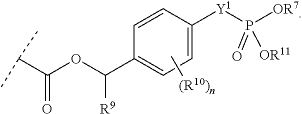

21. The compound according to any one of the preceding claims, wherein Y is —O— or Y is —N(H)— and R⁴ is

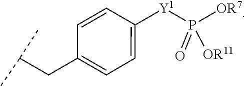

22. The compound according to any one of the preceding claims, wherein Y is —O— or Y is —N(H)— and R⁴ is

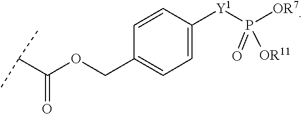

23. The compound according to any one of the preceding claims, wherein YR⁴ taken together is

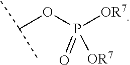

24. The compound according to any one of the preceding claims, wherein R⁵ is O.

25. The compound according to any one of the preceding claims, wherein R² is a linking group.

26. The compound according to any one of the preceding claims, wherein T is a protein that binds specifically to a cellular target.

27. The compound according to any one of the preceding claims, wherein the cellular target is an extracellular target.

28. The compound according to any one of the preceding claims, wherein binding of T to the extracellular target affects endocytosis of the compound.

29. The compound according to any one of the preceding claims, wherein T is an antibody.

30. The compound according to any one of the preceding claims, wherein T is an internalising antibody.

31. The compound according to any one of the preceding claims, wherein T is an antibody-derived antigen binding fragment.

32. The compound according to any one of the preceding claims, wherein T is a single-chain variable fragment.

33. The compound according to any one of the preceding claims, wherein T is a single-domain antibody.

34. The compound according to any one of the preceding claims, wherein T is a nanobody.

35. The compound according to any one of the preceding claims, wherein T is a DARPin.

36. The compound according to any one of the preceding claims, wherein T is a monobody.

37. The compound according to any one of the preceding claims, wherein T is a affibody.

38. The compound according to any one of the preceding claims, wherein T is a carbohydrate.

39. The compound according to any one of the preceding claims, wherein T is a oligonucleotide.

40. The compound according to any one of the preceding claims, wherein T is a lipid.

41. The compound according to any one of the preceding claims, wherein r is 1 to 12.

42. The compound according to any one of the preceding claims, wherein X is —S—.

43. The compound according to any one of the preceding claims, wherein X is >N—, ═N—, or —N(H)—.

44. The compound according to any one of the preceding claims, wherein X is —O—.

45. The compound according to any one of the preceding claims, wherein the compound is a prodrug.

46. The compound of formula I for use in medicine.

47. The compound of formula I for use in diagnostics.
